(19) United States
(12) Patent Application Publication
(10) Pub. No.: US 2024/0124555 A1
(43) Pub. Date: Apr. 18, 2024

(54) OPTIMIZED FACTOR VIII GENES

(71) Applicant: Bioverativ Therapeutics Inc., Waltham, MA (US)

(72) Inventors: Siyuan TAN, Lexington, MA (US); Tongyao LIU, Lexington, MA (US)

(21) Appl. No.: 18/364,103

(22) Filed: Aug. 2, 2023

Related U.S. Application Data
(62) Division of application No. 16/074,729, filed on Aug. 1, 2018, now Pat. No. 11,753,461.
(60) Provisional application No. 62/409,739, filed on Oct. 18, 2016.
(60) Provisional application No. 62/289,696, filed on Feb. 1, 2016.

(51) Int. Cl. C07K 14/755; A61K 48/00; A61P 7/04; C12N 7/00; C12N 15/86

(52) U.S. Cl. CPC C07K 14/755; A61K 48/0016; A61K 48/005; A61P 7/04; C12N 7/00; C12N 15/86; A61K 38/00; C07K 2319/30; C07K 2319/31; C12N 2740/16043; C12N 2800/22

(57) ABSTRACT

The present disclosure provides codon optimized Factor VIII sequences, vectors, and host cells comprising codon optimized Factor VIII sequences, polypeptides encoded by codon optimized Factor VIII sequences, and methods of producing such polypeptides. The present disclosure also provides methods of treating bleeding disorders such as hemophilia comprising administering to the subject a codon optimized Factor VIII nucleic acid sequence or the polypeptide encoded thereby.

### CROSS-REFERENCE TO RELATED APPLICATIONS

This application is a division of U.S. patent application Ser. No. 16/074,729, filed Aug. 1, 2018, which is a 35 U.S.C. § 371 filing of International Patent Application No. PCT/US2017/015879, filed Jan. 31, 2017, which claims priority to U.S. Provisional Patent Application Serial Nos. 62/409,739, filed Oct. 18, 2016, and 62/289,696, filed Feb. 1, 2016, the entire disclosures of which are hereby incorporated herein by reference.

### REFERENCE TO SEQUENCE LISTING SUBMITTED ELECTRONICALLY

The instant application contains a Sequence Listing which has been submitted electronically in XML format and is hereby incorporated by reference in its entirety. Said XML file, created on Aug. 1, 2023, is named 744757_SA9-452USDIV_ST26.xml and is 207,422 bytes in size.

### BACKGROUND OF THE DISCLOSURE

The blood coagulation pathway, in part, involves the formation of an enzymatic complex of Factor Villa (FVIIIa) and Factor IXa (FIXa) (Xase complex) on the surface of platelets. FIXa is a serine protease with relatively weak catalytic activity without its cofactor FVIIIa. The Xase complex cleaves Factor X (FX) into Factor Xa (FXa), which in turn interacts with Factor Va (FVa) to cleave prothrombin and generate thrombin. Hemophilia A is a bleeding disorder caused by mutations and/or deletions in the FVIII (FVIII) gene resulting in a deficiency of FVIII activity (Peyvandi et al. 2006). In some cases, patients have reduced levels of FVIII due to the presence of FVIII inhibitors, such as anti-FVIII antibodies.

Hemophilia A is characterized by spontaneous hemorrhage and excessive bleeding. Over time, the repeated bleeding into muscles and joints, which often begins in early childhood, results in hemophilic arthropathy and irreversible joint damage. This damage is progressive and can lead to severely limited mobility of joints, muscle atrophy and chronic pain (Rodriguez-Merchan, E. C., Semin. Thromb. Hemost. 29:87-96 (2003), which is herein incorporated by reference in its entirety).

The disease can be treated by replacement therapy targeting restoration of FVIII activity to 1 to 5% of normal levels to prevent spontaneous bleeding (see, e.g., Mannucci, P. M., et al., N. Engl. J. Med. 344:1773-9 (2001), herein incorporated by reference in its entirety). There are plasma-derived and recombinant FVIII products available to treat bleeding episodes on-demand or to prevent bleeding episodes from occurring by treating prophylactically. Based on the half-life of these products (10-12 hr) (White G. C., et al., Thromb. Haemost. 77:660-7 (1997); Morfini, M., Haemophilia 9 (suppl 1):94-99; discussion 100 (2003)), treatment regimens require frequent intravenous administration, commonly two to three times weekly for prophylaxis and one to three times daily for on-demand treatment (Manco-Johnson, M. J., et al., N. Engl. J. Med. 357:535-544 (2007)), each of which is incorporated herein by reference in its entirety. Such frequent administration is inconvenient and costly.

A major impediment in providing a low-cost recombinant FVIII protein to patients is the high cost of commercial production. FVIII protein expresses poorly in heterologous expression systems, two to three orders of magnitude lower than similarly sized proteins. (Lynch et al., Hum. Gene. Ther.; 4:259-72 (1993). The poor expression of FVIII is due in part to the presence of cis-acting elements in the FVIII coding sequence that inhibit FVIII expression, such as transcriptional silencer elements (Hoeben et al., Blood 85:2447-2454 (1995)), matrix attachment-like sequences (MARs) (Fallux et al., Mol. Cell. Biol. 16:4264-4272 (1996)), and transcriptional elongation inhibitory elements (Koeberl et al., Hum. Gene. Ther.; 6:469-479 (1995)).

Advances in our understanding of the biology of FVIII expression has led to the development of more potent FVIII variants. For instance, biochemical studies demonstrated that the FVIII B-domain was dispensable for FVIII cofactor activity. Deletion of the B-domain resulted in a 17-fold increase in mRNA levels over full-length wild-type FVIII and a 30% increase in secreted protein. (Toole et al., Proc Natl Acad Sci USA 83:5939-42 (1986)). This led to the development of B domain-deleted (BDD) FVIII protein concentrate, which is now widely used in the clinic. Recent studies, however, indicate that full length and BDD hFVIII misfold in the ER lumen, resulting in activation of the unfolded protein response (UPR) and apoptosis of murine hepatocytes.

Thus, there exists a need in the art for FVIII sequences that express efficiently in heterologous systems.

### SUMMARY OF THE DISCLOSURE

Disclosed are codon optimized nucleic acid molecules encoding a polypeptide with FVIII activity. In one aspect, the disclosure provides an isolated nucleic acid molecule comprising a nucleotide sequence which comprises a first nucleic acid sequence encoding an N-terminal portion of a Factor VIII (FVIII) polypeptide and a second nucleic acid sequence encoding a C-terminal portion of a FVIII polypeptide; wherein the first nucleic acid sequence has at least 85%, at least 90%, at least 95%, at least 96%, at least 97%, at least 98%, or at least 99% sequence identity to (i) nucleotides 58-2277 and 2320-1791 of SEQ ID NO: 3 or (ii) nucleotides 58-2277 and 2320-1791 of SEQ ID NO: 4; and wherein the N-terminal portion and the C-terminal portion together have a FVIII polypeptide activity.

The disclosure also provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 85%, at least 90%, at least 95%, at least 96%, at least 97%, at least 98%, or at least 99% sequence identity to (i) nucleotides 1792-2277 and 2320-4374 of SEQ ID NO: 5 or (ii) 1792-2277 and 2320-4374 of SEQ ID NO: 6; and wherein the N-terminal portion and the C-terminal portion together have a FVIII polypeptide activity.

In some embodiments, the disclosure provides an isolated nucleic acid molecule comprising a nucleotide sequence encoding a polypeptide with FVIII activity, wherein the nucleotide sequence comprises a nucleic acid sequence having at least 91%, at least 95%, at least 96%, at least 97%, at least 98%, or at least 99% sequence identity to nucleotides 58-2277 and 2320-4374 of SEQ ID NO: 1. In other embodiments, the disclosure provides an isolated nucleic acid molecule comprising a nucleotide sequence encoding a polypeptide with FVIII activity, wherein the nucleotide sequence comprises a nucleic acid sequence having at least about 94%, at least about 95%, at least 96%, at least 97%, at least 98%, or at least 99% sequence identity to nucleotides 58-2277 and 2320-4374 of SEQ ID NO: 2. In one embodiment, the disclosure provides an isolated nucleic acid molecule comprising a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58-2277 and 2320-4374 of SEQ ID NO: 70. In another embodiment, the disclosure provides an isolated nucleic acid molecule comprising a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58-2277 and 2320-4374 of SEQ ID NO: 71.

In some embodiments, the disclosure provides an isolated nucleic acid molecule comprising a nucleotide sequence encoding a polypeptide with FVIII activity, wherein the nucleotide sequence comprises a nucleic acid sequence having at least about 92%, at least about 93%, at least about 94%, at least about 95%, at least 96%, at least 97%, at least 98% or at least 99% sequence identity to nucleotides 58-2277 and 2320-4374 of SEQ ID NO: 3.

In some embodiments, the disclosure provides an isolated nucleic acid molecule comprising a nucleotide sequence encoding a polypeptide with FVIII activity, wherein the nucleotide sequence comprises a nucleic acid sequence having at least about 91%, at least about 92%, at least about 93%, at least about 94%, at least about 95%, at least 96%, at least 97%, at least 98% or at least 99% sequence identity to nucleotides 58-2277 and 2320-4374 of SEQ ID NO: 4.

In some embodiments, the disclosure provides an isolated nucleic acid molecule comprising a nucleotide sequence encoding a polypeptide with FVIII activity, wherein the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 5.

In some embodiments, the disclosure provides an isolated nucleic acid molecule comprising a nucleotide sequence encoding a polypeptide with FVIII activity, wherein the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 6.

In some embodiments, the disclosure provides a method of increasing expression of a polypeptide with FVIII activity in a subject comprising administering an isolated nucleic acid molecule or a vector disclosed herein to a subject in need thereof, wherein the expression of the polypeptide is increased relative to a reference nucleic acid molecule comprising SEQ ID NO: 16 or the vector comprising the reference nucleic acid molecule.

In some embodiments, the disclosure provides a method of treating a bleeding disorder comprising: administering to a subject in need thereof a nucleic acid molecule, a vector, or a polypeptide disclosed herein.

### EMBODIMENTS

E1. An isolated nucleic acid molecule comprising a nucleotide sequence which comprises a first nucleic acid sequence encoding an N-terminal portion of a Factor VIII (FVIII) polypeptide and a second nucleic acid sequence encoding a C-terminal portion of a FVIII polypeptide; wherein the first nucleic acid sequence has at least 85%, at least 90%, at least 95%, at least 96%, at least 97%, at least 98%, or at least 99% sequence identity to (i) nucleotides 58-2277 and 2320-1791 of SEQ ID NO: 3 or (ii) nucleotides 58-2277 and 2320-1791 of SEQ ID NO: 4; and wherein the N-terminal portion and the C-terminal portion together have a FVIII polypeptide activity.

E2. The isolated nucleic acid molecule of E1, wherein the first nucleotide sequence comprises nucleotides 58-1791 of SEQ ID NO: 3 or nucleotides 58-1791 of SEQ ID NO: 4.

E3. The isolated nucleic acid molecule of E1 or E2, wherein the second nucleotide sequence has at least 60%, at least 70%, at least 80%, at least 90%, at least 95%, at least 96%, at least 97%, at least 98%, or at least 99% sequence identity to nucleotides 1792-2277 and 2320-4374 of SEQ ID NO: 3 or 1792-2277 and 2320-4374 of SEQ ID NO: 4.

E4. The isolated nucleic acid molecule of E1 or E2, wherein the second nucleotide sequence comprises nucleotides 1792-2277 and 2320-4374 of SEQ ID NO: 3 or 1792-2277 and 2320-4374 of SEQ ID NO: 4.

E5. An isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 85%, at least 90%, at least 95%, at least 96%, at least 97%, at least 98%, or at least 99% sequence identity to (i) nucleotides 1792-2277 and 2320-4374 of SEQ ID NO: 5 or (ii) 1792-2277 and 2320-4374 of SEQ ID NO: 6; and wherein the N-terminal portion and the C-terminal portion together have a FVIII polypeptide activity.

E6. The isolated nucleic acid molecule of E5, wherein the second nucleic acid sequence comprises nucleotides 1792-2277 and 2320-4374 of SEQ ID NO: 5 or 1792-2277 and 2320-4374 of SEQ ID NO: 6.

E7. The isolated nucleic acid molecule of E5 or E6, wherein the first nucleic acid sequence has at least 60%, at least 70%, at least 80%, at least 90%, at least 95%, at least 96%, at least 97%, at least 98%, or at least 99% sequence identity to nucleotides 58-1791 of SEQ ID NO: 5 or nucleotides 58-1791 of SEQ ID NO: 6.

E8. The isolated nucleic acid molecule of E5 or E6, wherein the first nucleic acid sequence comprises nucleotides 58-1791 of SEQ ID NO: 5 or nucleotides 58-1791 of SEQ ID NO: 6.

E9. An isolated nucleic acid molecule comprising a nucleotide sequence encoding a polypeptide with FVIII activity, wherein the nucleotide sequence comprises a nucleic acid sequence having at least 91%, at least 95%, at least 96%, at least 97%, at least 98%, or at least 99% sequence identity to nucleotides 58-2277 and 2320-4374 of SEQ ID NO: 1.

E10. The isolated nucleic acid molecule of E9, wherein the nucleotide sequence comprises nucleotides 58-2277 and 2320-4374 of SEQ ID NO: 1.

E11. An isolated nucleic acid molecule comprising a nucleotide sequence encoding a polypeptide with FVIII activity, wherein the nucleotide sequence comprises a nucleic acid sequence having at least about 94%, at least about 95%, at least 96%, at least 97%, at least 98%, or at least 99% sequence identity to nucleotides 58-2277 and 2320-4374 of SEQ ID NO: 2.

E12. The isolated nucleic acid molecule of E10, wherein the nucleotide sequence comprises nucleotides 58-2277 and 2320-4374 of SEQ ID NO: 2.

E13. An isolated nucleic acid molecule comprising a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58-2277 and 2320-4374 of SEQ ID NO: 70.

E14. The isolated nucleic acid molecule of E13, wherein the nucleotide sequence comprises nucleotides 58-2277 and 2320-4374 of SEQ ID NO: 70.

E15. An isolated nucleic acid molecule comprising a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58-2277 and 2320-4374 of SEQ ID NO: 71.

E16. The isolated nucleic acid molecule of E15, wherein the nucleotide sequence comprises nucleotides 58-2277 and 2320-4374 of SEQ ID NO: 71.

E17. An isolated nucleic acid molecule comprising a nucleotide sequence encoding a polypeptide with FVIII activity, wherein the nucleotide sequence comprises a nucleic acid sequence having at least about 92%, at least about 93%, at least about 94%, at least about 95%, at least 96%, at least 97%, at least 98% or at least 99% sequence identity to nucleotides 58-2277 and 2320-4374 of SEQ ID NO: 3.

E18. The isolated nucleic acid molecule of E17, wherein the nucleotide sequence comprises nucleotides 58-2277 and 2320-4374 of SEQ ID NO: 3.

E19. An isolated nucleic acid molecule comprising a nucleotide sequence encoding a polypeptide with FVIII activity, wherein the nucleotide sequence comprises a nucleic acid sequence having at least about 91%, at least about 92%, at least about 93%, at least about 94%, at least about 95%, at least 96%, at least 97%, at least 98% or at least 99% sequence identity to nucleotides 58-2277 and 2320-4374 of SEQ ID NO: 4.

E20. The isolated nucleic acid molecule of E19, wherein the nucleotide sequence comprises nucleotides 58-2277 and 2320-4374 of SEQ ID NO: 4.

E21. An isolated nucleic acid molecule comprising a nucleotide sequence encoding a polypeptide with FVIII activity, wherein the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 5.

E22. The isolated nucleic acid molecule of E21, wherein the nucleotide sequence comprises nucleotides 58-2277 and 2320-4374 of SEQ ID NO: 5.

E23. An isolated nucleic acid molecule comprising a nucleotide sequence encoding a polypeptide with FVIII activity, wherein the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 6.

E24. The isolated nucleic acid molecule of E23, wherein the nucleotide sequence comprises nucleotides 58-2277 and 2320-4374 of SEQ ID NO: 6.

E25. The isolated nucleic acid molecule of any one of E1 to E24, wherein the nucleotide sequence further comprises a nucleic acid sequence encoding a signal peptide.

E26. The isolated nucleic acid molecule of E25, wherein the signal peptide is a FVIII signal peptide.

E27. The isolated nucleic acid molecule of E25 or E26, wherein the nucleic acid sequence encoding a signal peptide is codon optimized.

E28. The isolated nucleic acid molecule of any one of E25 to E27, wherein the nucleic acid sequence encoding a signal peptide has at least 60%, at least 70%, at least 80%, at least 90%, at least 95%, at least 96%, at least 97%, at least 98%, at least 99%, or 100% sequence identity to (i) nucleotides 1 to 57 of SEQ ID NO: 1; (ii) nucleotides 1 to 57 of SEQ ID NO: 2; (iii) nucleotides 1 to 57 of SEQ ID NO: 3; (iv) nucleotides 1 to 57 of SEQ ID NO: 4; (v) nucleotides 1 to 57 of SEQ ID NO: 5; (vi) nucleotides 1 to 57 of SEQ ID NO: 6; (vii) nucleotides 1 to 57 of SEQ ID NO: 70; (viii) nucleotides 1 to 57 of SEQ ID NO: 71; or (ix) nucleotides 1 to 57 of SEQ ID NO: 68.

E29. The isolated nucleic acid molecule of any one of E1 to E28, wherein the nucleic acid molecule comprises one or more property selected from the group consisting of:
(a) the human codon adaptation index the nucleic acid molecule or a portion thereof is increased relative to SEQ ID NO: 16;
(b) the frequency of optimal codons of the nucleotide sequence or a portion thereof is increased relative to SEQ ID NO:16;
(c) the nucleotide sequence or a portion thereof contains a higher percentage of G/C nucleotides compared to the percentage of G/C nucleotides in SEQ ID NO: 16;
(d) the relative synonymous codon usage of the nucleotide sequence or a portion thereof is increased relative to SEQ ID NO: 16;
(e) the effective number of codons of the nucleotide sequence or a portion thereof is reduced relative SEQ ID NO: 16;
(f) the nucleotide sequence contains fewer MARS/ARS sequences (SEQ ID NOs: 21 and 22) relative to SEQ ID NO: 16;
(g) the nucleotide sequence contains fewer destabilizing elements (SEQ ID NOs: 23 and 24) relative to SEQ ID NO: 16; and
(h) any combination thereof.

E30. The isolated nucleic acid molecule of any one of E1 to E29 further comprising a heterologous nucleotide sequence.

E31. The isolated nucleic acid molecule of E30, wherein the heterologous nucleotide sequence encodes a heterologous amino acid sequence that is a half-life extender.

E32. The isolated nucleic acid molecule of E30 or E31, wherein the heterologous amino acid sequence is an immunoglobulin constant region or a portion thereof, transferrin, albumin, or a PAS sequence.

E33. The isolated nucleic acid molecule of E30 or E31, wherein the heterologous amino acid sequence is an Fc region.

E34. The isolated nucleic acid molecule of any one of E30 to E33, wherein the heterologous amino acid sequence is linked to the N-terminus or the C-terminus of the amino acid sequence encoded by the nucleotide sequence or inserted between two amino acids in the amino acid sequence encoded by the nucleotide sequence.

E35. The isolated nucleic acid molecule of E34, wherein the heterologous amino acid sequence is inserted between two amino acids at one or more insertion site selected from Table 3.

E36. The isolated nucleic acid molecule of any one of E30 to E35, which encodes a monomer-dimer hybrid molecule comprising FVIII.

E37. The isolated nucleic acid molecule of any one of E1 to E36, wherein the FVIII polypeptide is a full length FVIII or a B domain deleted FVIII.

E38. The isolated nucleic acid molecule of any one of any one of E1 to E37, operably linked to at least one transcription control sequence.

E39. A vector comprising the nucleic acid molecule of any one of E1 to E38.

E40. The vector of E39, wherein the vector is a viral vector.

E41. A host cell comprising the nucleic acid molecule of any one of E1 to E32 or the vector of E39 or E40.

E42. The host cell of E41, wherein the host cell is selected from the group consisting of: a CHO cell, a HEK293 cell, a BHK21 cell, a PER.C6 cell, a NS0 cell, and a CAP cell.

E43. A polypeptide encoded by the nucleic acid molecule of any one of E1 to E37 or the vector of E39 or 40 or produced by the host cell of E41 or E42.

E44. A method of producing a polypeptide with FVIII activity, comprising: culturing the host cell of E41 or E42 under conditions whereby a polypeptide with FVIII activity is produced; and, recovering the polypeptide with FVIII activity.

E45. The method of E44, wherein the expression of the polypeptide with FVIII activity is increased relative to a host cell cultured under the same conditions comprising a reference nucleotide sequence comprising SEQ ID NO: 16.

E46. A method of increasing expression of a polypeptide with FVIII activity in a subject comprising administering the isolated nucleic acid molecule of any one of E1 to E38 or the vector of E39 or E40 to a subject in need thereof, wherein the expression of the polypeptide is increased relative to a reference nucleic acid molecule comprising SEQ ID NO: 16 or the vector comprising the reference nucleic acid molecule.

E47. A method of increasing expression of a polypeptide with FVIII activity comprising culturing the host cell of E41 or E42 under conditions whereby a polypeptide with FVIII activity is expressed by the nucleic acid molecule, wherein the expression of the polypeptide with FVIII activity is increased relative to a host cell cultured under the same conditions comprising a reference nucleic acid sequence comprising SEQ ID NO: 16.

E48. The method of any one of E44 to E47, wherein the expression of the FVIII polypeptide is increased by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 50-fold, at least about 60-fold, at least about 70-fold, at least about 80-fold, at least about 90-fold, or at least about 100-fold.

E49. A method of improving yield of a polypeptide with FVIII activity comprising culturing the host cell of E41 or E42 under conditions whereby a polypeptide with FVIII activity is produced by the nucleic acid molecule, wherein the yield of polypeptide with FVIII activity is increased relative to a host cell cultured under the same conditions comprising a reference nucleic acid sequence comprising SEQ ID NO: 16.

E50. A method of treating a bleeding disorder comprising: administering to a subject in need thereof a nucleic acid molecule of any one of E1 to E38, the vector of E39 or E40, or the polypeptide of E43.

E51. The method of E50, wherein the bleeding disorder is characterized by a deficiency in FVIII.

E52. The method of E50, wherein the bleeding disorder is hemophilia.

E53. The method of E50, wherein the bleeding disorder is hemophilia A.

E54. The method of any one of E50 to E53, wherein plasma FVIII activity at 24 hours post administration is increased relative to a subject administered a reference nucleic acid molecule comprising SEQ ID NO: 16, a vector comprising the reference nucleic acid molecule, or a polypeptide encoded by the reference nucleic acid molecule.

E55. The method of E54, wherein the plasma FVIII activity is increased by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 50-fold, at least about 60-fold, at least about 70-fold, at least about 80-fold, at least about 90-fold, or at least about 100-fold.

E56. The vector of E39 or 40, wherein the vector is a lentiviral vector.

E57. The vector of any one of E39, E40, and E56 further comprising a tissue specific promoter, a tissue specific enhancer, or both a tissue specific promoter and a tissue specific enhancer.

E58. The vector of E57, wherein the tissue specific promoter and/or the tissue specific enhancer selectively enhances expression of the transgene in liver cells.

E59. The vector of E57 or E58, wherein the tissue specific promoter comprises a promoter sequence selected from the group consisting of a mouse thyretin promoter (mTTR), an endogenous human factor VIII promoter (F8), human alpha-1-antitrypsin promoter (hAAT), human albumin minimal promoter, and mouse albumin promoter.

E60. The vector of any one of E57 to E59, wherein the tissue specific promoter comprises mTTR promoter.

E61. A method of treating a bleeding disorder comprising: administering to a subject in need thereof the vector of any one of E56 to E60.

### BRIEF DESCRIPTION OF THE DRAWINGS

FIGS. 1A-1J provide the codon optimized nucleotide sequences encoding B domain-deleted Factor VIII (SEQ ID NO: 17). FIG. 1A shows the nucleotide sequence of coFVIII-3 (SEQ ID NO:1). FIG. 1B shows the nucleotide sequence of coFVIII-4 (SEQ ID NO: 2). FIG. 1C shows the nucleotide sequence of coFVIII-5 (SEQ ID NO: 70). FIG. 1D shows the nucleotide sequence of coFVIII-6 (SEQ ID NO: 71). FIG. 1E shows the nucleotide sequence of coFVIII-52 (SEQ ID NO: 3). FIG. 1F shows the nucleotide sequence of coFVIII-62 (SEQ ID NO: 4). FIG. 1G shows the nucleotide sequence of coFVIII-25 (SEQ ID NO: 5). FIG. 1H shows the nucleotide sequence of coFVIII-26 (SEQ ID NO: 6). FIGS. 1I and 1J show the nucleotide and amino acid sequences, respectively, of B domain-deleted (BDD-FVIII) (SEQ ID NOs: 16 and 17, respectively).

FIGS. 2A-2J show codon usage bias adjustments in the codon optimized nucleotide sequences encoding BDD-FVIII. FIG. 2A shows the relative frequency of codons in the wild-type nucleotide sequence (before codon optimization) encoding BDD-FVIII, e.g., non-optimized BDD-FVIII. The human codon adaptation index (CAI) of the non-optimized BDD-FVIII sequence is 74%. FIG. 2B shows the relative frequency of codons in the coFVIII-1 variant sequence, which has a human CAI of 88%. FIG. 2C shows the relative frequency of codons in the coFVIII-3 variant sequence, which has a human CAI of 91%. FIG. 2D shows the relative frequency of codons in the coFVIII-4 variant sequence, which has a human CAI of 97%. FIG. 2E shows the relative frequency of codons in the coFVIII-5 variant sequence, which has a human CAI of 83%. FIG. 2F shows the relative frequency of codons in the coFVIII-6 variant sequence, which has a human CAI of 83%. FIG. 2G shows the relative frequency of codons in the coFVIII-52 variant sequence, which has a human CAI of 91%. FIG. 2H shows the relative frequency of codons in the coFVIII-62 variant sequence, which has a human CAI of 91%. FIG. 2I shows the relative frequency of codons in the coFVIII-25 variant sequence, which has a human CAI of 88%. FIG. 2J shows the relative frequency of codons in the coFVIII-26 variant sequence, which has a human CAI of 88%.

FIG. 3 provides a plasmid map of FVIII-303, which comprises coFVIII-1 in a pcDNA3 backbone under the control of the ET-enhanced transthyretin promoter, which is positioned upstream of the coFVIII-1 translation start site and which comprises a synthetic enhancer, an mTIR enhancer, and an mTIR promoter.

FIG. 4 shows a graphical representation of FVIII plasma activity in HemA mice following hydrodynamic injection of 5 μg FVIII-303 (coFVIII-1; circles) or 5 μg FVIII-311 (BDD-FVIII; squares). FVIII plasma activity was determined by a FVIII specific chromogenic assay at 24, 48, and 72 hours post-injection. The relative activity levels at 72 hours, normalized to the expression level of FVIII-311, are shown.

FIG. 5 shows a plasmid map of pLV-coFVIII-52, which comprises coFVIII-52 in a lentiviral plasmid under the control of an ET promoter, which is positioned upstream of the coFVIII-52 translation start site and which comprises a synthetic enhancer, an mTTR enhancer, and an mTTR promoter.

FIGS. 6A-6C show graphical representations of FVIII plasma activity in HemA mice following hydrodynamic injection of various FVIII encoding nucleotides. FVIII plasma activity was determined by a FVIII specific chromogenic assay at 24, 48, and 72 hours post-injection. FIG. 6A shows FVIII plasma activity in HemA mice following hydrodynamic injection of 5 μg LV-coFVIII-1 (filled circles), 5 μg LV-coFVIII-3 (triangles), 5 μg LV-coFVIII-4 (inverted triangles), 5 μg LV-coFVIII-5 (diamonds), or 5 μg LV-coFVIII-6 (open circles). FIG. 6B shows FVIII plasma activity in HemA mice following hydrodynamic injection of 5 μg LV-coFVIII-1 (circles), 5 μg LV-coFVIII-25 (triangles), or 5 μg LV-coFVIII-26 (inverted triangles). FIG. 6C shows FVIII plasma activity in HemA mice following hydrodynamic injection of 20 μg LV-2116 (non-codon optimized (WT) BDD-FVIII nucleotide sequence; open circles), 20 μg LV-coFVIII-1 (triangles), 20 μg LV-coFVIII-52 (squares), or 20 μg LV-coFVIII-62 (filled circles). The relative activity levels at 72 hours are shown for each plasmid, normalized to the expression levels of LV-coFVIII-1 (FIGS. 6A, 6B, and 6C) and/or LV-2116 (FIG. 6C), as indicated.

FIG. 7 shows plasma FVIII activity in HemA mice 24 days after injection with 1E8 TU/mouse lentiviral vector comprising coFVIII-1, coFVIII-5, coFVIII-52, coFVIII-6, or coFVIII-62 as compared with the LV-2116 (BDD-FVIII) control, and as measured by a FVIII-specific chromogenic assay. Error bars indicate standard deviations.

FIGS. 8A-8C provide the various codon optimized nucleotide sequences encoding BDD-FVIII fused to an XTEN. FIG. 8A shows the nucleotide sequence of coFVIII-52-XTEN (SEQ ID NO: 19), wherein a nucleotide sequence encoding an XTEN having 144 amino acids (“XTEN₁₄₄”; SEQ ID NO: 18; underlined) is inserted within the coFVIII-52 nucleotide sequence. FIG. 8B shows the nucleotide sequence of coFVIII-1-XTEN (SEQ ID NO: 20), wherein a nucleotide sequence encoding an XTEN having 144 amino acid (“XTEN₁₄₄”; SEQ ID NO: 18; underlined) is inserted within the coFVIII-1 nucleotide sequence. FIG. 8C shows the nucleotide sequence of coFVIII-6-XTEN (SEQ ID NO: 72), wherein a nucleotide sequence encoding an XTEN having 144 amino acid (“XTEN₁₄₄”; SEQ ID NO: 18; underlined) is inserted within the coFVIII-6 nucleotide sequence (e.g., amino acid residue 745 corresponding to mature FVIII sequence).

FIG. 9 provides a plasmid map of pLV-coFVIII-52-XTEN, which comprises coFVIII-52-XTEN in a lentiviral vector under the control of the ET promoter. Lentiviral vectors comprising each of the remaining codon optimized nucleic acid molecules encoding a polypeptide with FVIII activity, as described herein, were constructed in the same manner as pLV-coFVIII-52-XTEN, in which the same XTEN sequence was inserted to replace the B-domain of FVIII.

FIGS. 10A and 10B show FVIII activity in HemA mice following injection with plasmid DNA (FIG. 10A) or lentiviral vector (FIG. 10B) comprising the various codon optimized nucleotide sequences encoding BDD-FVIII. FIG. 10A shows a graphical representation of FVIII plasma activity in HemA mice following hydrodynamic injection with 5 μg FVIII-311 (non-codon optimized, BDD-FVIII encoding nucleotide sequence; squares), 5 μg FVIII-303 (coFVIII-1; small circles), or FVIII-306 (coFVIII-1-XTEN₁₄₄; large circles). The relative activity at 72 hours, normalized to FVIII-311, is shown for each plasmid. FIG. 10B shows plasma FVIII activity in HemA mice 21 days after injection with 1E8 TU/mouse of lentiviral vector comprising coFVIII-52 or coFVIII-52-XTEN as compared with the LV-2116 (BDD-FVIII) control, and as measured by a FVIII-specific chromogenic assay. Error bars indicate standard deviations.

FIG. 11A shows the amino acid sequence of full-length mature human factor VIII.

FIG. 11B shows the amino acid sequence of full length human von Willebrand Factor (SEQ ID NO: 44). FIGS. 11C and 11D show the amino acid and nucleotide sequences, respectively, of an XTEN polypeptide having 42 amino acids (XTEN AE42-4; SEQ ID NOs: 46 and 47, respectively). The amino acid sequences of various XTEN polypeptides having 144 amino acids are shown in FIGS. 11E, 11G, 11I, 11K, 11M, 110, 11Q, 11S, 11U, and 11W (SEQ ID NOs: 48, 50, 52, 54, 56, 58, 60, 62, 64, and 66, respectively), and the corresponding nucleotide sequences are shown in FIGS. 11F, 11H, 11J, 11L, 11N, 11P, 11R, 11T, 11V, and 11X (SEQ ID NOs. 49, 51, 53, 55, 57, 59, 61, 63, 65, and 67, respectively). FIG. 11Y shows the nucleotide sequence of an ET promoter (SEQ ID NO: 69). FIG. 11Z shows the nucleotide sequence for coFVIII-1 (SEQ ID NO: 68) (see International Publication No. WO 2014/127215, SEQ ID NO: 1).

FIG. 12A is a graphic representation of FVIII plasma activity (IU/mL) in 14-day-old HemA mice following IV administration of about 1.5 E10 TU/kg LV-wtBDD-FVIII (circles), LV-coFVIII-6 (squares), or LV-coFVIII-6XTEN (triangles). FIG. 12B is a graphic representation of vector copy number (VCN) 150 days after treatment of 14-day-old HemA mice administered by IV about 1.5 E10 TU/kg of lentiviral vectors expressing wtBDD-FVIII, coFVIII-1, coFVIII-3, coFVIII-4, coFVIII-5, coFVIII-6, coFVIII-52, coFVIII-62, coFVIII-25, or coFVIII-26. FIG. 12C is a graphic representation of FVIII plasma activity (IU/mL) 21 days after treatment of 14-day-old HemA mice administered by IV about 1.5 E10 TU/kg of lentiviral vectors expressing wtBDD-FVIII, coFVIII-1, coFVIII-3, coFVIII-4, coFVIII-5, coFVIII-6, coFVIII-52, coFVIII-62, coFVIII-25, or coFVIII-26.

FIGS. 13A and 13B are graphic representations that illustrate the FVIII plasma activity levels (FIG. 13A) and anti-FVIII antibody levels (FIG. 13B) in five HemA mice treated with a lentivirus expressing the coFVIII-5 variant. Fourteen-day-old HemA littermates were administered approximately 1.5 E10 TU/kg of a lentivirus expressing the coFVIII-5 variant by intravenous injection. Each mouse is designated by a number (i.e., 1, 2, 3, 4, and 5; FIGS. 13A and 13B).

FIG. 14 is a graphic representation of the correlation between LV-FVIII expression level, as evidenced by FVIII plasma activity at 21 days post lentiviral treatment, and the presence of anti-FVIII antibodies. Each data point corresponds to a single HemA mouse. Each mouse received a 1.5 E10 TU/kg dose by intravenous injection of a lentivirus expressing one of the coFVIII variants disclosed herein. Horizontal lines indicate the average FVIII plasma activity.

FIG. 15 is a graphic representation of the correlation between vector copy number (VCN) per cell at 150 days post lentiviral treatment and the presence of anti-FVIII antibodies. Each data point corresponds to a single HemA mouse. Each mouse received a 1.5 E10 TU/kg dose by intravenous injection of a lentivirus expressing one of the coFVIII variants disclosed herein. Horizontal lines indicate the average VCN.

FIGS. 16A and 16B are graphic representations that illustrate the FVIII plasma activity levels (FIG. 16A) and anti-FVIII antibody levels (FIG. 16B) in two HemA mice (coFVIII-52-A and coFVIII-52-B) treated with a lentivirus expressing the coFVIII-52 variant. Fourteen-day-old HemA littermates were administered approximately 1.5 E10 TU/kg of a lentivirus expressing the coFVIII-52 variant by intravenous injection. FIGS. 16C and 16D are images showing RNA in situ hybridization staining for FVIII expression (dark staining) in liver tissue collected from the coFVIII-52-A (FIG. 16C) and coFVIII-52-B (FIG. 16D) mice of FIGS. 16A and 16B.

FIG. 17 is a graphic representation that shows long-term FVIII expression in HemA neonate mice treated with a lentivirus expressing a wild-type B domain deleted FVIII (wtBDD-FVIII; triangles), coFVIII-52XTEN (circles), or coFVIII-6XTEN (inverted triangle) variant. Neonatal HemA mice were administered by intravenous injection approximately 1.5 A10 TU/kg of a lentivirus expressing wtBDD-FVIII, coFVIII-52XTEN, or coFVIII-6XTEN. FVIII plasma activity was measured over approximately 16 weeks.

FIG. 18 is a graphic representation of the circulating FVIII level in HemA dog neonates (S3 or K4) after administration of 1.3×10⁹ transducing units/kg lentiviral vector comprising a nucleotide encoding Factor VIII fused to XTEN (SEQ ID NO: 72; LV-coFVIII-6-XTEN). Squares connected by a solid line represent aPTT-S3 samples, and triangles connected by a dashed line represent aPTT-K4 samples. The y-axis shows the plasma FVIII activity as the percent of normal, wherein normal human FVIII activity is 100%. The x-axis shows the days post-lentivirus treatment, wherein lentivirus treatment is administered at day 0.

FIGS. 19A-19C are graphical representations of whole blood hemostasis as monitored by rotational thromboelastometry (ROTEM) assay for a naïve HemA dog (FIG. 19A), dog S3 at 2 weeks post-lentivirus treatment (FIG. 19B), and dog K4 at 2 weeks post-lentivirus treatment (FIG. 19C). Clotting time (CT) is shown as seconds (s), clot formation time (CFT) is shown as seconds (s), alpha angle (α) is shown as degrees (°), amplitude 5 minutes after CT (A5) is shown as millimeters (mm), amplitude 20 minutes after CT (A20) is shown as millimeters (mm), and maximum clot firmness (MCF) is shown as millimeters (mm) for each of FIGS. 19A-19C. FIG. 19D is a table summarizing the normal range for each of the parameters CT, CFT, α, A5, A2, and MCF displayed in FIGS. 19A-19C.

### DETAILED DESCRIPTION OF THE DISCLOSURE

The present disclosure describes codon-optimized genes encoding polypeptides with Factor VIII (FVIII) activity. The present disclosure is directed to codon optimized nucleic acid molecules encoding polypeptides with Factor VIII activity, vectors and host cells comprising optimized nucleic acid molecules, polypeptides encoded by optimized nucleic acid molecules, and methods of producing such polypeptides. The present disclosure is also directed to methods of treating bleeding disorders such as hemophilia comprising administering to the subject an optimized Factor VIII nucleic acid sequence, a vector comprising the optimized nucleic acid sequence, or the polypeptide encoded thereby. The present disclosure meets an important need in the art by providing optimized Factor VIII sequences that demonstrate increased expression in host cells, improved yield of Factor VIII protein in methods to produce recombinant Factor VIII, and potentially result in greater therapeutic efficacy when used in gene therapy methods. In certain embodiments, the disclosure describes an isolated nucleic acid molecule comprising a nucleotide sequence which has sequence homology to a nucleotide sequence selected from SEQ ID NOs: 1-14, 70, and 71.

Exemplary constructs of the disclosure are illustrated in the accompanying Figures and sequence listing. In order to provide a clear understanding of the specification and claims, the following definitions are provided below.

### I. Definitions

It is to be noted that the term “a” or “an” entity refers to one or more of that entity: for example, “a nucleotide sequence” is understood to represent one or more nucleotide sequences. As such, the terms “a” (or “an”), “one or more,” and “at least one” can be used interchangeably herein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “isolated” for the purposes of the present disclosure designates a biological material (cell, polypeptide, polynucleotide, or a fragment, variant, or derivative thereof) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.” No particular level of purification is required. Recombinantly produced polypeptides and proteins expressed in host cells are considered isolated for the purpose of the disclosure, as are native or recombinant polypeptides which have been separated, fractionated, or partially or substantially purified by any suitable technique.

“Nucleic acids,” “nucleic acid molecules,” “oligonucleotide,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region, or comprise two or more coding regions.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In certain embodiments, a native signal peptide or a functional derivative of that sequence that retains the ability to direct the secretion of the polypeptide that is operably associated with it. Alternatively, a heterologous mammalian signal peptide, e.g., a human tissue plasminogen activator (TPA) or mouse ß-glucuronidase signal peptide, or a functional derivative thereof, can be used.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites and stem-loop structures. If a coding region is intended for expression in a eukaryotic cell, a polyadenylation signal and transcription termination sequence will usually be located 3′ to the coding sequence.

A polynucleotide which encodes a gene product, e.g., a polypeptide, can include a promoter and/or other expression (e.g., transcription or translation) control elements operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

“Transcriptional control sequences” refer to DNA regulatory sequences, such as promoters, enhancers, terminators, and the like, that provide for the expression of a coding sequence in a host cell. A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit ß-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like. Selectable marker genes can also be considered reporter genes.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

The terms “restriction endonuclease” and “restriction enzyme” are used interchangeably and refer to an enzyme that binds and cuts within a specific nucleotide sequence within double stranded DNA.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

Eukaryotic viral vectors that can be used include, but are not limited to, adenovirus vectors, retrovirus vectors, adeno-associated virus vectors, poxvirus, e.g., vaccinia virus vectors, baculovirus vectors, or herpesvirus vectors. Non-viral vectors include plasmids, liposomes, electrically charged lipids (cytofectins), DNA-protein complexes, and biopolymers.

A “cloning vector” refers to a “replicon,” which is a unit length of a nucleic acid that replicates sequentially and which comprises an origin of replication, such as a plasmid, phage or cosmid, to which another nucleic acid segment can be attached so as to bring about the replication of the attached segment. Certain cloning vectors are capable of replication in one cell type, e.g., bacteria and expression in another, e.g., eukaryotic cells. Cloning vectors typically comprise one or more sequences that can be used for selection of cells comprising the vector and/or one or more multiple cloning sites for insertion of nucleic acid sequences of interest.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter.

“Culture,” “to culture” and “culturing,” as used herein, means to incubate cells under in vitro conditions that allow for cell growth or division or to maintain cells in a living state. “Cultured cells,” as used herein, means cells that are propagated in vitro.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

The term “amino acid” includes alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr orY); and valine (Val or V). Non-traditional amino acids are also within the scope of the disclosure and include norleucine, omithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, C). These amino acids can participate in hydrophobic interactions and electrostatic interactions. As used herein, the term “charged amino acid” includes amino acids that can have non-zero net charge on their side chains (e.g., R, K, H, E, D). These amino acids can participate in hydrophobic interactions and electrostatic interactions.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., FcRn binding affinity for an FcRn binding domain or Fc variant, coagulation activity for an FVIII variant, or FVIII binding activity for the VWF fragment) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case can be, as determined by the match between strings of such sequences. “Identity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity are codified in publicly available computer programs. Sequence alignments and percent identity calculations can be performed using sequence analysis software such as the Megalign program of the LASERGENE bioinformatics computing suite (DNASTAR Inc., Madison, WI), the GCG suite of programs (Wisconsin Package Version 9.0, Genetics Computer Group (GCG), Madison, WI), BLASTP, BLASTN, BLASTX (Altschul et al., J. Mol. Biol. 215:403 (1990)), and DNASTAR (DNASTAR, Inc. 1228 S. Park St. Madison, WI 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized. For the purposes of determining percent identity between an optimized BDD FVIII sequence of the disclosure and a reference sequence, only nucleotides in the reference sequence corresponding to nucleotides in the optimized BDD FVIII sequence of the disclosure are used to calculate percent identity. For example, when comparing a full length FVIII nucleotide sequence containing the B domain to an optimized B domain deleted (BDD) FVIII nucleotide sequence of the disclosure, the portion of the alignment including the A1, A2, A3, C1, and C2 domain will be used to calculate percent identity. The nucleotides in the portion of the full length FVIII sequence encoding the B domain (which will result in a large “gap” in the alignment) will not be counted as a mismatch. In addition, in determining percent identity between an optimized BDD FVIII sequence of the disclosure, or a designated portion thereof (e.g., nucleotides 58-2277 and 2320-4374 of SEQ ID NO:3), and a reference sequence, percent identity will be calculated by aligning dividing the number of matched nucleotides by the total number of nucleotides in the complete sequence of the optimized BDD-FVIII sequence, or a designated portion thereof, as recited herein.

As used herein, “nucleotides corresponding to nucleotides in the optimized BDD FVIII sequence of the disclosure” are identified by alignment of the optimized BDD FVIII sequence of the disclosure to maximize the identity to the reference FVIII sequence. The number used to identify an equivalent amino acid in a reference FVIII sequence is based on the number used to identify the corresponding amino acid in the optimized BDD FVIII sequence of the disclosure.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the disclosure with an Ig Fc domain. A fusion protein is created, for example, by chemical synthesis, or by creating and translating a polynucleotide in which the peptide regions are encoded in the desired relationship. A chimeric protein can further comprises a second amino acid sequence associated with the first amino acid sequence by a covalent, non-peptide bond or a non-covalent bond.

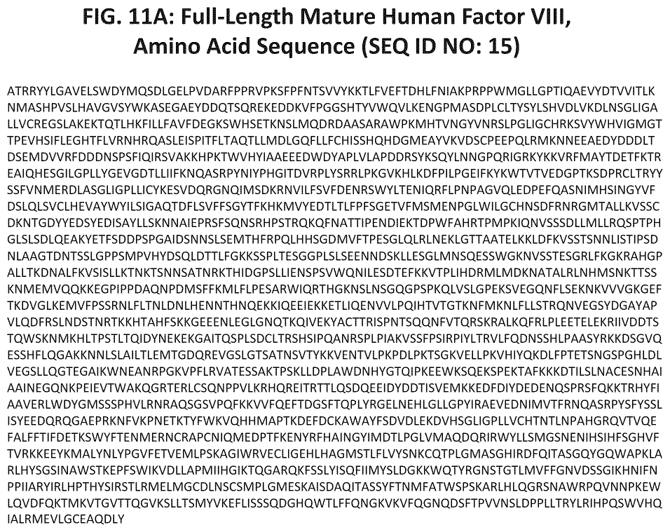

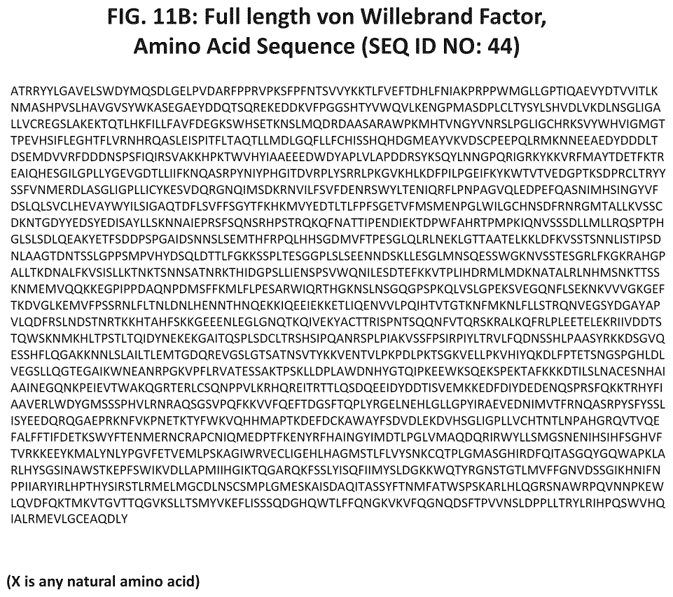

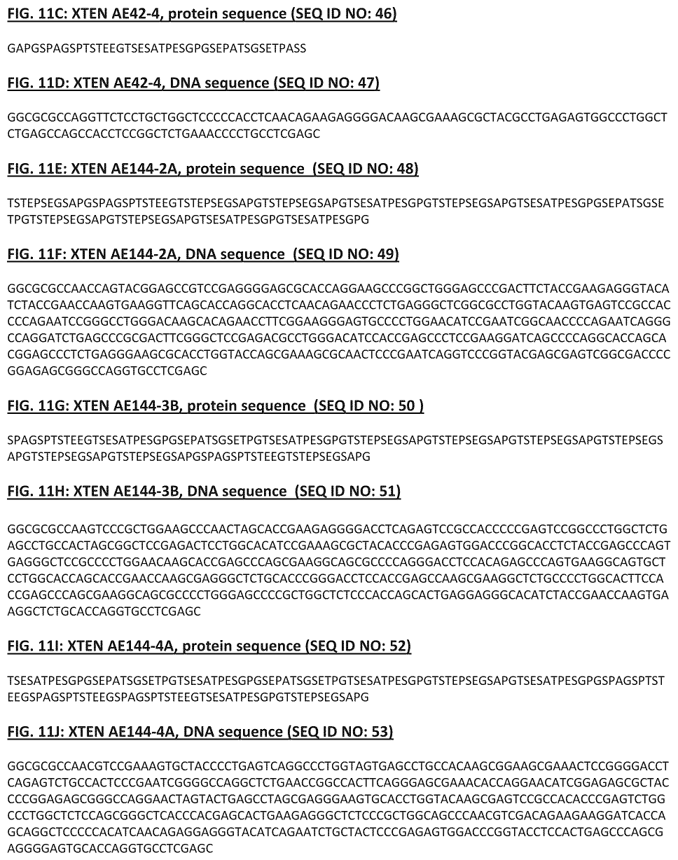

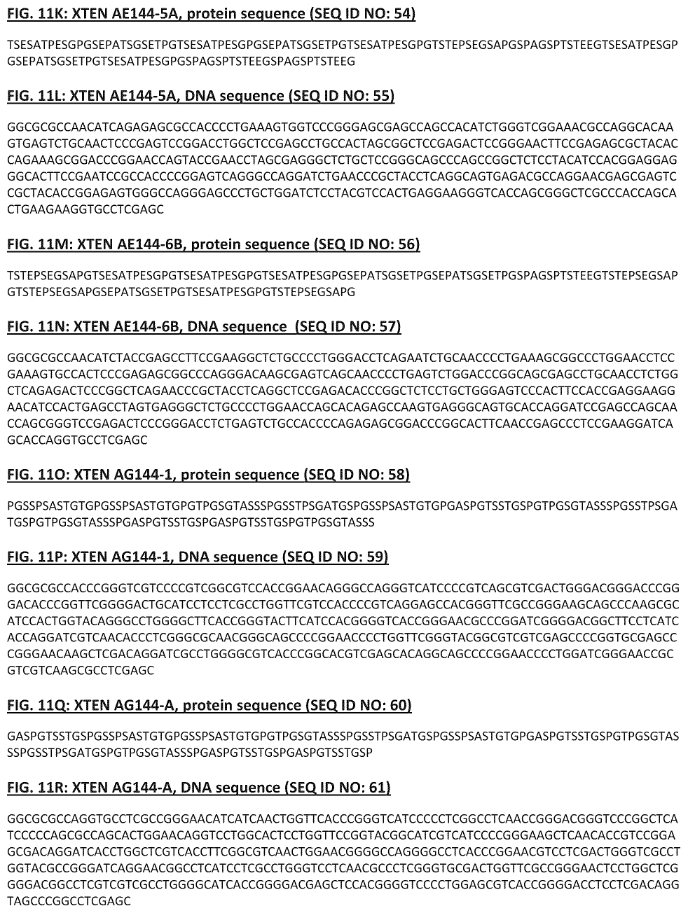

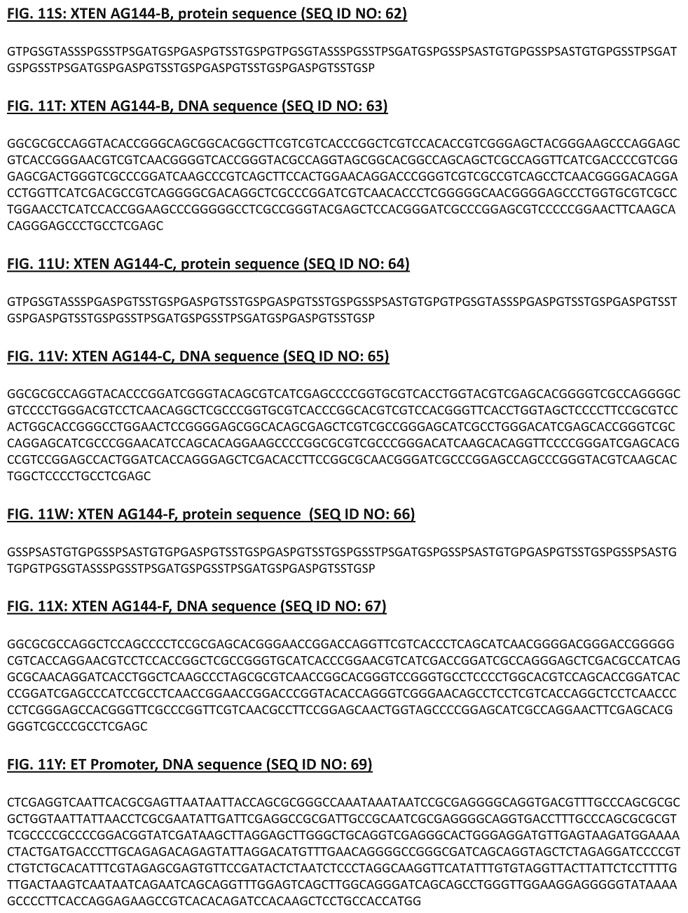

As used herein, the term “insertion site” refers to a position in a FVIII polypeptide, or fragment, variant, or derivative thereof, which is immediately upstream of the position at which a heterologous moiety can be inserted. An “insertion site” is specified as a number, the number being the number of the amino acid in mature native FVIII (SEQ ID NO: 15; FIG. 11A) to which the insertion site corresponds, which is immediately N-terminal to the position of the insertion. For example, the phrase “a3 comprises a heterologous moiety at an insertion site which corresponds to amino acid 1656 of SEQ ID NO: 15” indicates that the heterologous moiety is located between two amino acids corresponding to amino acid 1656 and amino acid 1657 of SEQ ID NO: 15.

The phrase “immediately downstream of an amino acid” as used herein refers to position right next to the terminal carboxyl group of the amino acid. Similarly, the phrase “immediately upstream of an amino acid” refers to the position right next to the terminal amine group of the amino acid.

The terms “inserted,” “is inserted,” “inserted into” or grammatically related terms, as used herein refers to the position of a heterologous moiety in a recombinant FVIII polypeptide, relative to the analogous position in native mature human FVIII. As used herein the terms refer to the characteristics of the recombinant FVIII polypeptide relative to native mature human FVIII, and do not indicate, imply or infer any methods or process by which the recombinant FVIII polypeptide was made.

As used herein, the term “half-life” refers to a biological half-life of a particular polypeptide in vivo. Half-life can be represented by the time required for half the quantity administered to a subject to be cleared from the circulation and/or other tissues in the animal. When a clearance curve of a given polypeptide is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, FVIII and chimeric proteins comprising FVIII are monophasic, and thus do not have an alpha phase, but just the single beta phase. Therefore, in certain embodiments, the term half-life as used herein refers to the half-life of the polypeptide in the β-phase.

The term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

As used herein the term “associated with” refers to a covalent or non-covalent bond formed between a first amino acid chain and a second amino acid chain. In one embodiment, the term “associated with” means a covalent, non-peptide bond or a non-covalent bond. This association can be indicated by a colon, i.e., (:). In another embodiment, it means a covalent bond except a peptide bond. For example, the amino acid cysteine comprises a thiol group that can form a disulfide bond or bridge with a thiol group on a second cysteine residue. In most naturally occurring IgG molecules, the CH1 and CL regions are associated by a disulfide bond and the two heavy chains are associated by two disulfide bonds at positions corresponding to 239 and 242 using the Kabat numbering system (position 226 or 229, EU numbering system). Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, an hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity.

The term “monomer-dimer hybrid” used herein refers to a chimeric protein comprising a first polypeptide chain and a second polypeptide chain, which are associated with each other by a disulfide bond, wherein the first chain comprises a clotting factor, e.g., Factor VIII, and a first Fc region and the second chain comprises, consists essentially of, or consists of a second Fc region without the clotting factor. The monomer-dimer hybrid construct thus is a hybrid comprising a monomer aspect having only one clotting factor and a dimer aspect having two Fc regions.

Hemostasis, as used herein, means the stopping or slowing of bleeding or hemorrhage; or the stopping or slowing of blood flow through a blood vessel or body part.

Hemostatic disorder, as used herein, means a genetically inherited or acquired condition characterized by a tendency to hemorrhage, either spontaneously or as a result of trauma, due to an impaired ability or inability to form a fibrin clot. Examples of such disorders include the hemophilias. The three main forms are hemophilia A (factor VIII deficiency), hemophilia B (factor IX deficiency or “Christmas disease”) and hemophilia C (factor XI deficiency, mild bleeding tendency). Other hemostatic disorders include, e.g., von Willebrand disease, Factor XI deficiency (PTA deficiency), Factor XII deficiency, deficiencies or structural abnormalities in fibrinogen, prothrombin, Factor V, Factor VII, Factor X or factor XIII, Bernard-Soulier syndrome, which is a defect or deficiency in GPIb. GPIb, the receptor for vWF, can be defective and lead to lack of primary clot formation (primary hemostasis) and increased bleeding tendency), and thrombasthenia of Glanzman and Naegeli (Glanzmann thrombasthenia). In liver failure (acute and chronic forms), there is insufficient production of coagulation factors by the liver; this can increase bleeding risk.

The isolated nucleic acid molecules, isolated polypeptides, or vectors comprising the isolated nucleic acid molecule of the disclosure can be used prophylactically. As used herein the term “prophylactic treatment” refers to the administration of a molecule prior to a bleeding episode. In one embodiment, the subject in need of a general hemostatic agent is undergoing, or is about to undergo, surgery. A polynucleotide, polypeptide, or vector of the disclosure can be administered prior to or after surgery as a prophylactic. The polynucleotide, polypeptide, or vector of the disclosure can be administered during or after surgery to control an acute bleeding episode. The surgery can include, but is not limited to, liver transplantation, liver resection, dental procedures, or stem cell transplantation.

The isolated nucleic acid molecules, isolated polypeptides, or vectors of the disclosure are also used for on-demand treatment. The term “on-demand treatment” refers to the administration of an isolated nucleic acid molecule, isolated polypeptide, or vector in response to symptoms of a bleeding episode or before an activity that can cause bleeding. In one aspect, the on-demand treatment can be given to a subject when bleeding starts, such as after an injury, or when bleeding is expected, such as before surgery. In another aspect, the on-demand treatment can be given prior to activities that increase the risk of bleeding, such as contact sports.

As used herein the term “acute bleeding” refers to a bleeding episode regardless of the underlying cause. For example, a subject can have trauma, uremia, a hereditary bleeding disorder (e.g., factor VII deficiency) a platelet disorder, or resistance owing to the development of antibodies to clotting factors.

Treat, treatment, treating, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition. In one embodiment, the term “treating” or “treatment” means maintaining a FVIII trough level at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, or 20 IU/dL in a subject by administering an isolated nucleic acid molecule, isolated polypeptide or vector of the disclosure. In another embodiment, treating or treatment means maintaining a FVIII trough level between about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL. Treatment or treating of a disease or condition can also include maintaining FVIII activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, or 20% of the FVIII activity in a non-hemophiliac subject. The minimum trough level required for treatment can be measured by one or more known methods and can be adjusted (increased or decreased) for each person.

“Administering,” as used herein, means to give a pharmaceutically acceptable Factor VIII-encoding nucleic acid molecule, Factor VIII polypeptide, or vector comprising a Factor VIII-encoding nucleic acid molecule of the disclosure to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. The nucleic acid molecules, polypeptides, and vectors can be administered as part of a pharmaceutical composition comprising at least one excipient.

As used herein, the phrase “subject in need thereof” includes subjects, such as mammalian subjects, that would benefit from administration of a nucleic acid molecule, a polypeptide, or vector of the disclosure, e.g., to improve hemostasis. In one embodiment, the subjects include, but are not limited to, individuals with hemophilia. In another embodiment, the subjects include, but are not limited to, the individuals who have developed a FVIII inhibitor and thus are in need of a bypass therapy. The subject can be an adult or a minor (e.g., under 12 years old).

As used herein, the term “clotting factor,” refers to molecules, or analogs thereof, naturally occurring or recombinantly produced which prevent or decrease the duration of a bleeding episode in a subject. In other words, it means molecules having pro-clotting activity, i.e., are responsible for the conversion of fibrinogen into a mesh of insoluble fibrin causing the blood to coagulate or clot. An “activatable clotting factor” is a clotting factor in an inactive form (e.g., in its zymogen form) that is capable of being converted to an active form.

Clotting activity, as used herein, means the ability to participate in a cascade of biochemical reactions that culminates in the formation of a fibrin clot and/or reduces the severity, duration or frequency of hemorrhage or bleeding episode.

As used herein the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

As used herein, the term “heterologous nucleotide sequence” refers to a nucleotide sequence that does not naturally occur with a given polynucleotide sequence. In one embodiment, the heterologous nucleotide sequence encodes a polypeptide capable of extending the half-life of FVIII. In another embodiment, the heterologous nucleotide sequence encodes a polypeptide that increases the hydrodynamic radius of FVIII. In other embodiments, the heterologous nucleotide sequence encodes a polypeptide that improves one or more pharmacokinetic properties of FVIII without significantly affecting its biological activity or function (e.g., its procoagulant activity). In some embodiments, FVIII is linked or connected to the polypeptide encoded by the heterologous nucleotide sequence by a linker. Non-limiting examples of polypeptide moieties encoded by heterologous nucleotide sequences include an immunoglobulin constant region or a portion thereof, albumin or a fragment thereof, an albumin-binding moiety, a transferrin, the PAS polypeptides of U.S. Pat Application No. 20100292130, a HAP sequence, transferrin or a fragment thereof, the C-terminal peptide (CTP) of the β subunit of human chorionic gonadotropin, albumin-binding small molecule, an XTEN sequence, FcRn binding moieties (e.g., complete Fc regions or portions thereof which bind to FcRn), single chain Fc regions (ScFc regions, e.g., as described in US 2008/0260738, WO 2008/012543, or WO 2008/1439545), polyglycine linkers, polyserine linkers, peptides and short polypeptides of 6-40 amino acids of two types of amino acids selected from glycine (G), alanine (A), serine (S), threonine (T), glutamate (E) and proline (P) with varying degrees of secondary structure from less than 50% to greater than 50%, amongst others, or two or more combinations thereof. In some embodiments, the polypeptide encoded by the heterologous nucleotide sequence is linked to a non-polypeptide moiety. Non-limiting examples of the non-polypeptide moieties include polyethylene glycol (PEG), albumin-binding small molecules, polysialic acid, hydroxyethyl starch (HES), a derivative thereof, or any combinations thereof.

As used herein, the term “Fc region” is defined as the portion of a polypeptide which corresponds to the Fc region of native Ig, i.e., as formed by the dimeric association of the respective Fc domains of its two heavy chains. A native Fc region forms a homodimer with another Fc region. In contrast, the term “genetically-fused Fc region” or “single-chain Fc region” (scFc region), as used herein, refers to a synthetic dimeric Fc region comprised of Fc domains genetically linked within a single polypeptide chain (i.e., encoded in a single contiguous genetic sequence).

In one embodiment, the “Fc region” refers to the portion of a single Ig heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The Fc region of an Ig constant region, depending on the Ig isotype can include the CH2, CH3, and CH4 domains, as well as the hinge region. Chimeric proteins comprising an Fc region of an Ig bestow several desirable properties on a chimeric protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1), which are incorporated herein by reference in their entireties.

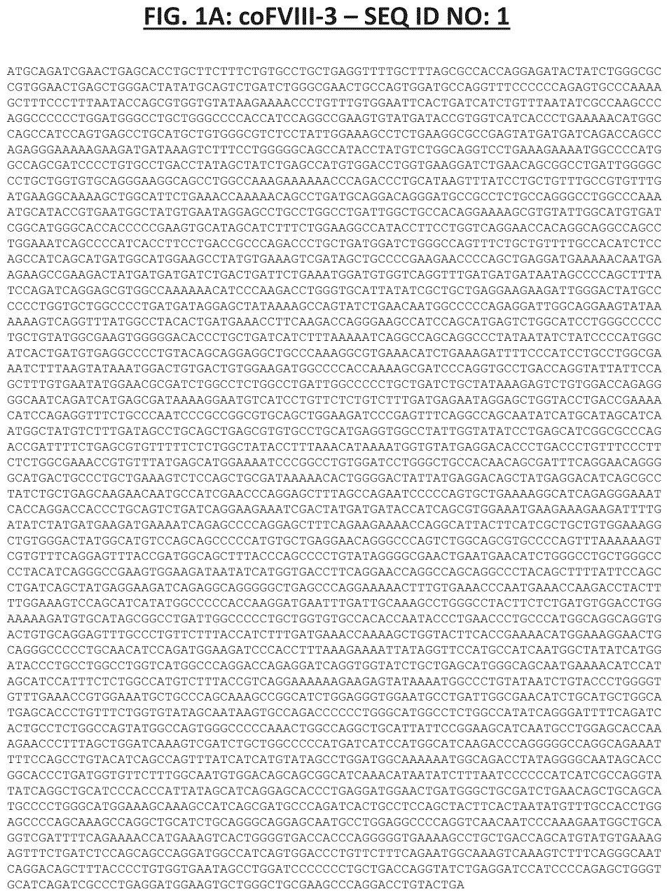

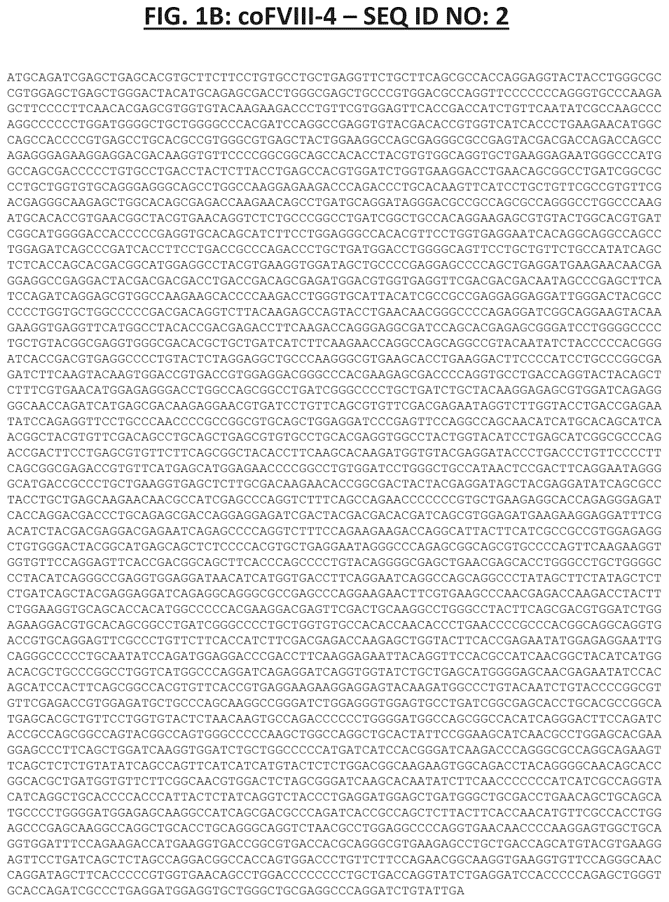

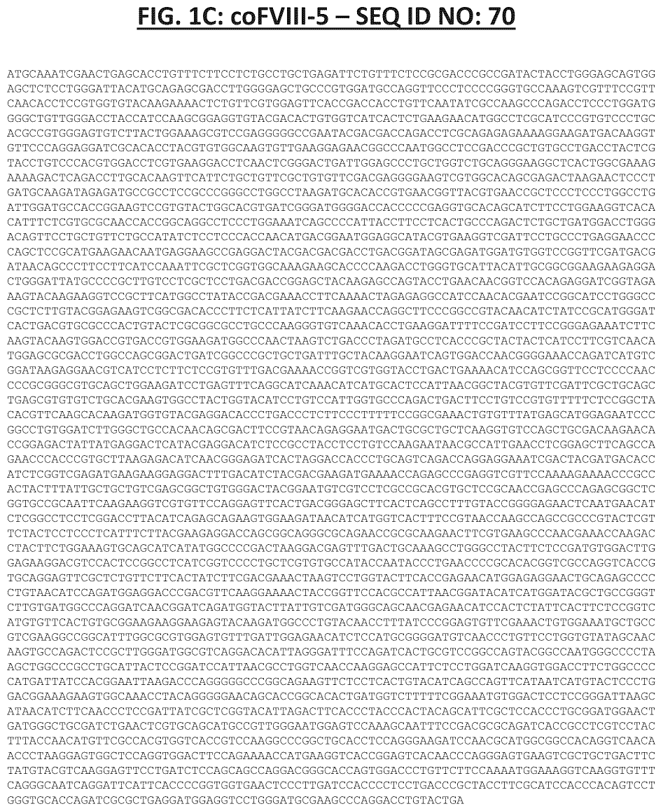

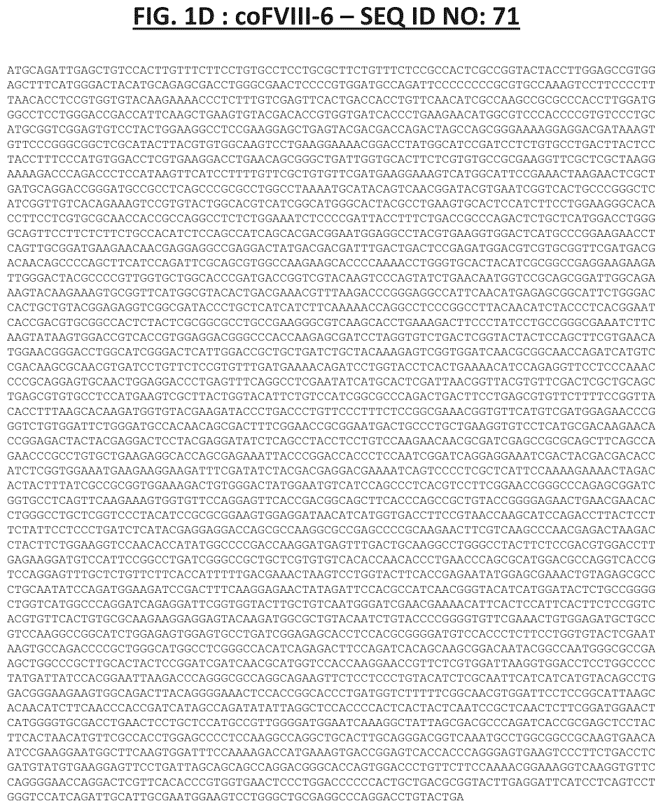

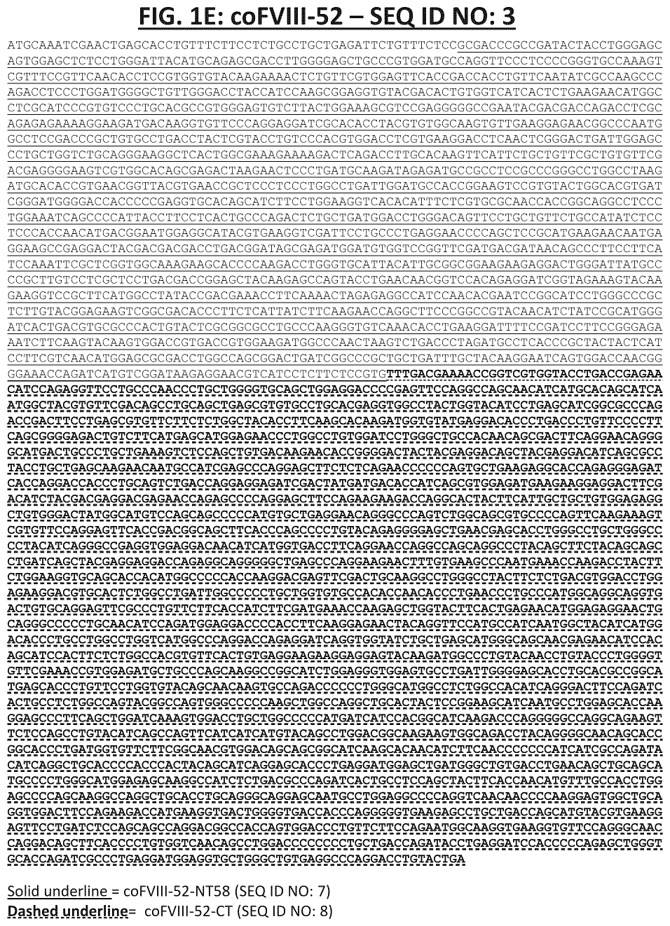

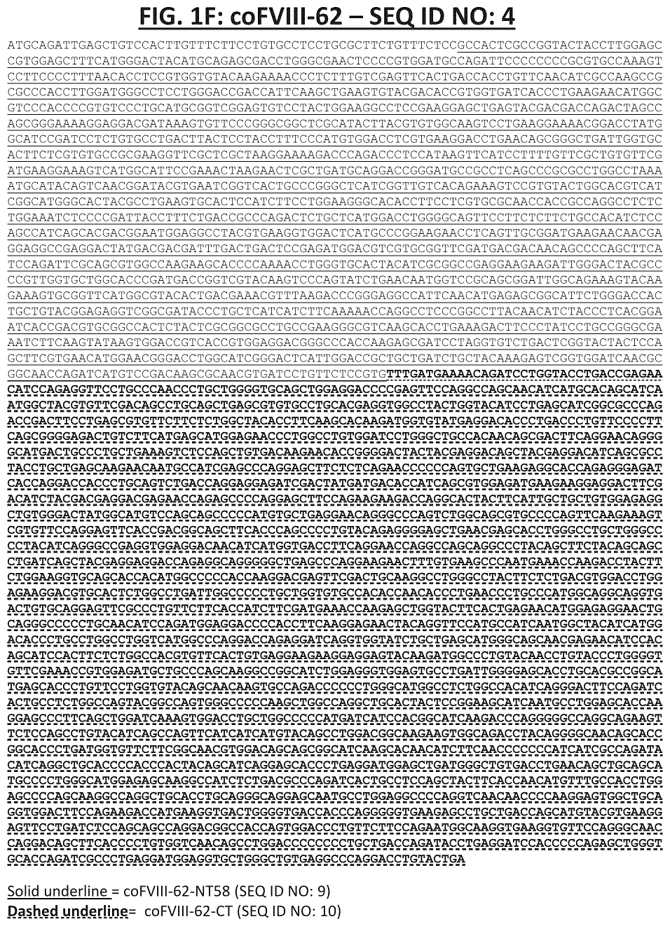

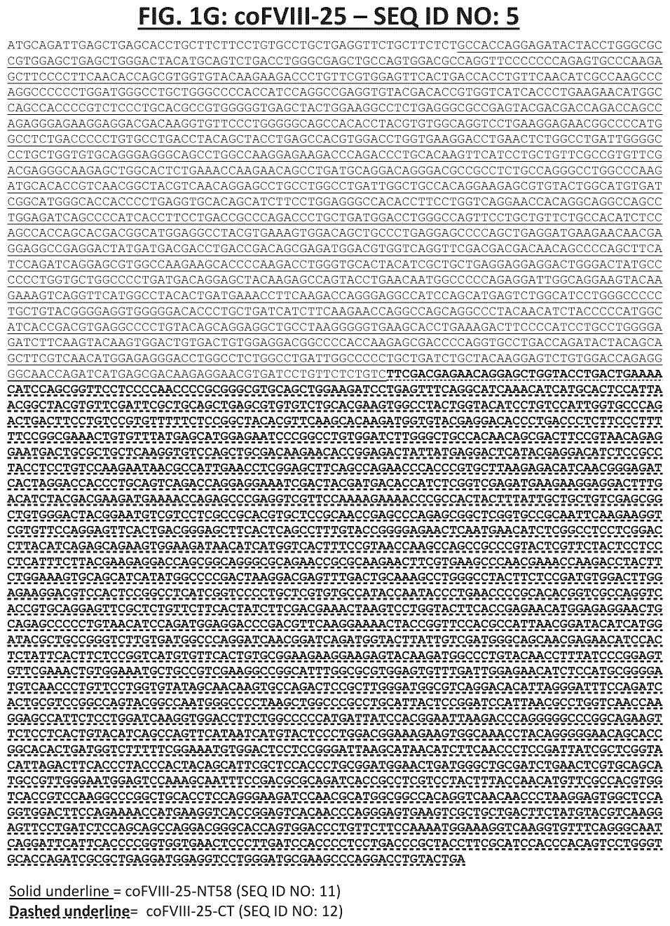

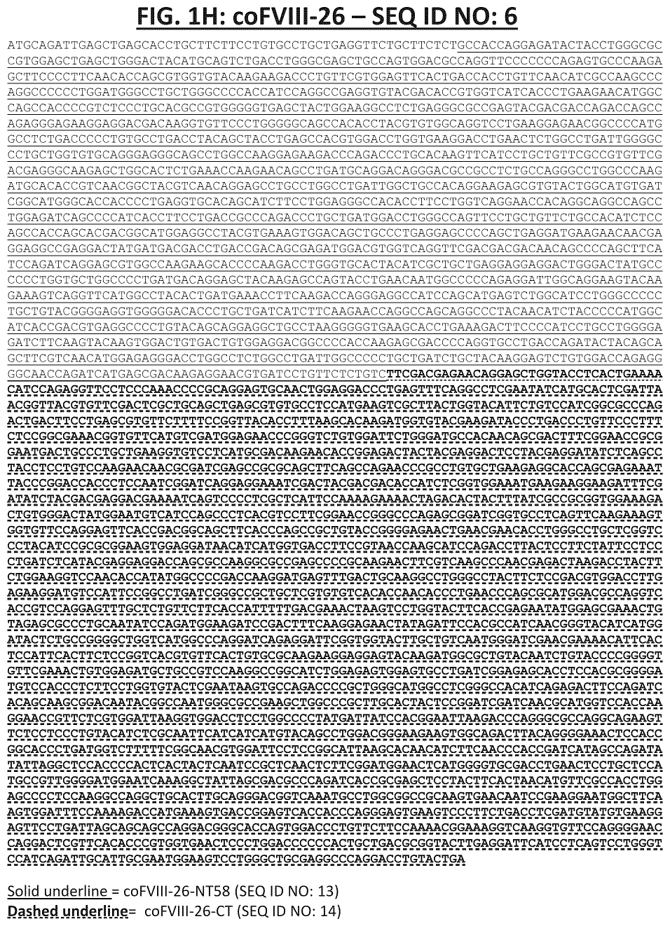

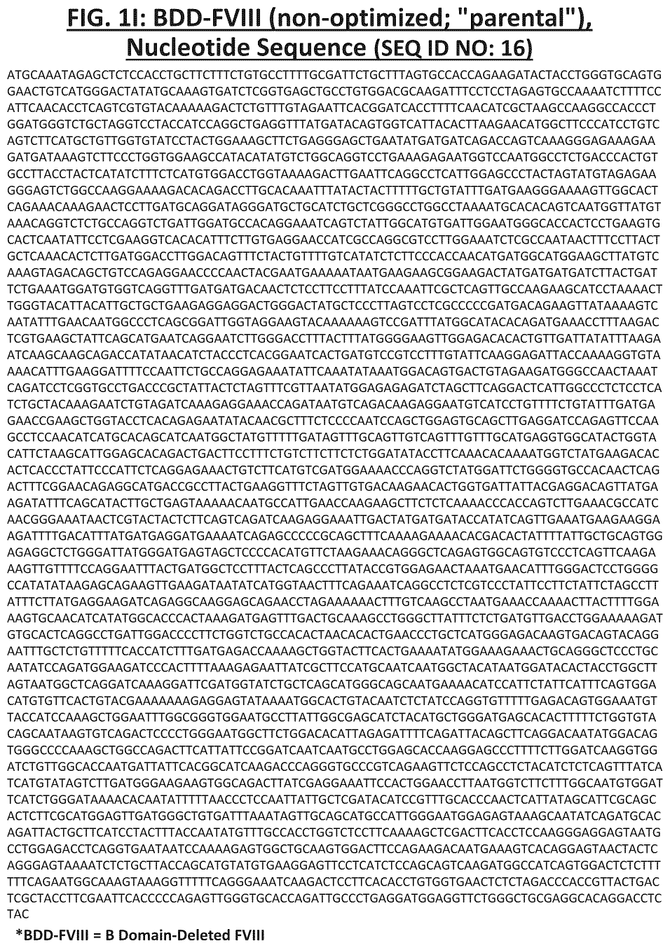

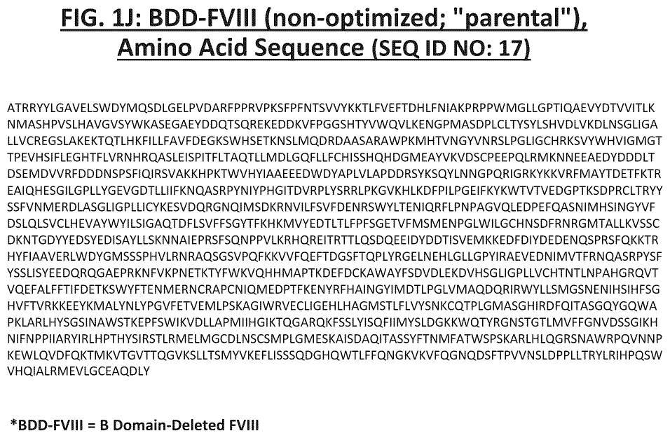

A “reference nucleotide sequence,” when used herein as a comparison to a nucleotide sequence of the disclosure, is a polynucleotide sequence essentially identical to the nucleotide sequence of the disclosure except that the portions corresponding to FVIII sequence are not optimized. For example, the reference nucleotide sequence for a nucleic acid molecule consisting of the codon optimized BDD FVIII of SEQ ID NO: 1 and a heterologous nucleotide sequence that encodes a single chain Fc region linked to SEQ ID NO: 1 at its 3′ end is a nucleic acid molecule consisting of the original (or “parent”) BDD FVIII of SEQ ID NO: 16 (FIG. 1I) and the identical heterologous nucleotide sequence that encodes a single chain Fc region linked to SEQ ID NO: 16 at its 3′ end.

A “codon adaptation index,” as used herein, refers to a measure of codon usage bias. A codon adaptation index (CAI) measures the deviation of a given protein coding gene sequence with respect to a reference set of genes (Sharp PM and Li WH, Nucleic Acids Res. 15(3):1281-95 (1987)). CAI is calculated by determining the geometric mean of the weight associated to each codon over the length of the gene sequence (measured in codons):

$$CAI = exp ( 1 / L {\sum }_{l = 1}^{L} ln ( {w}_{i} ( l ) ) ) , \qquad ( I )$$

For each amino acid, the weight of each of its codons, in CAI, is computed as the ratio between the observed frequency of the codon (fi) and the frequency of the synonymous codon (fj) for that amino acid:

$$\begin{matrix}Formula 2 & \\ {w}_{i} = \frac{{f}_{i}}{max ( {f}_{i} )} ij ∈ [ synonymous condons for amino acid ] & ( II )\end{matrix}$$

As used herein, the term “optimized,” with regard to nucleotide sequences, refers to a polynucleotide sequence that encodes a polypeptide, wherein the polynucleotide sequence has been mutated to enhance a property of that polynucleotide sequence. In some embodiments, the optimization is done to increase transcription levels, increase translation levels, increase steady-state mRNA levels, increase or decrease the binding of regulatory proteins such as general transcription factors, increase or decrease splicing, or increase the yield of the polypeptide produced by the polynucleotide sequence. Examples of changes that can be made to a polynucleotide sequence to optimize it include codon optimization, G/C content optimization, removal of repeat sequences, removal of AT rich elements, removal of cryptic splice sites, removal of cis-acting elements that repress transcription or translation, adding or removing poly-T or poly-A sequences, adding sequences around the transcription start site that enhance transcription, such as Kozak consensus sequences, removal of sequences that could form stem loop structures, removal of destabilizing sequences, and two or more combinations thereof.

### Polynucleotide Sequence Encoding FVIII Protein

In some embodiments, the present disclosure is directed to codon optimized nucleic acid molecules encoding a polypeptide with FVIII activity. In some embodiments, the polynucleotide encodes a full-length FVIII polypeptide. In other embodiments, the nucleic acid molecule encodes a B domain-deleted (BDD) FVIII polypeptide, wherein all or a portion of the B domain of FVIII is deleted. In one particular embodiment, the nucleic acid molecule encodes a polypeptide comprising an amino acid sequence having at least about 80%, at least about 85%, at least about 86%, at least about 87%, at least about 88%, at least about 89%, at least about 90%, at least about 95%, at least about 96%, at least about 97%, at least about 98%, or at least about 99% sequence identity to SEQ ID NO: 17 (FIG. 1J) or a fragment thereof. In one embodiment, the nucleic acid molecule encodes a polypeptide having the amino acid sequence of SEQ ID NO: 17 or a fragment thereof.

In some embodiments, the nucleic acid molecule of the disclosure encodes a FVIII polypeptide comprising a signal peptide or a fragment thereof. In other embodiments, the nucleic acid molecule encodes a FVIII polypeptide which lacks a signal peptide. In some embodiments, the signal peptide comprises amino acids 1-19 of SEQ ID NO: 17.

In some embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least 85%, at least 90%, at least 95%, at least 96%, at least 97%, at least 98%, or at least 99% sequence identity to (i) nucleotides 58-1791 of SEQ ID NO: 3 or (ii) nucleotides 58-1791 of SEQ ID NO: 4; and wherein the N-terminal portion and the C-terminal portion together have a FVIII polypeptide activity. In one particular embodiment, the first nucleic acid sequence has at least 85%, at least 90%, at least 95%, at least 96%, at least 97%, at least 98%, or at least 99% sequence identity to nucleotides 58-1791 of SEQ ID NO: 3. In another embodiment, the first nucleic acid sequence has at least 85%, at least 90%, at least 95%, at least 96%, at least 97%, at least 98%, or at least 99% sequence identity to nucleotides 58-1791 of SEQ ID NO: 4. In other embodiments, the first nucleotide sequence comprises nucleotides 58-1791 of SEQ ID NO: 3 or nucleotides 58-1791 of SEQ ID NO: 4.

In other embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least 85%, at least 90%, at least 95%, at least 96%, at least 97%, at least 98%, or at least 99% sequence identity to (i) nucleotides 1-1791 of SEQ ID NO: 3 or (ii) nucleotides 1-1791 of SEQ ID NO: 4; and wherein the N-terminal portion and the C-terminal portion together have a FVIII polypeptide activity. In one embodiment, the first nucleotide sequence comprises nucleotides 1-1791 of SEQ ID NO: 3 or nucleotides 1-1791 of SEQ ID NO: 4. In another embodiment, the second nucleotide sequence has at least 60%, at least 70%, at least 80%, at least 90%, at least 95%, at least 96%, at least 97%, at least 98%, or at least 99% sequence identity to nucleotides 1792-4374 of SEQ ID NO: 3 or 1792-4374 of SEQ ID NO: 4. In one particular embodiment, the second nucleotide sequence comprises nucleotides 1792-4374 of SEQ ID NO: 3 or 1792-4374 of SEQ ID NO: 4. In still another embodiment, the second nucleotide sequence has at least 60%, at least 70%, at least 80%, at least 90%, at least 95%, at least 96%, at least 97%, at least 98%, or at least 99% sequence identity to nucleotides 1792-2277 and 2320-4374 of SEQ ID NO: 3 or 1792-2277 and 2320-4374 of SEQ ID NO: 4 (i.e., nucleotides 1792-4374 of SEQ ID NO: 3 or 1792-4374 of SEQ ID NO: 4 without the nucleotides encoding the B domain or B domain fragment). In one particular embodiment, the second nucleotide sequence comprises nucleotides 1792-2277 and 2320-4374 of SEQ ID NO: 3 or 1792-2277 and 2320-4374 of SEQ ID NO: 4 (i.e., nucleotides 1792-4374 of SEQ ID NO: 3 or 1792-4374 of SEQ ID NO: 4 without the nucleotides encoding the B domain or B domain fragment).

In some embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 85%, at least 90%, at least 95%, at least 96%, at least 97%, at least 98%, or at least 99% sequence identity to (i) nucleotides 1792-4374 of SEQ ID NO: 5 or (ii) 1792-4374 of SEQ ID NO: 6; and wherein the N-terminal portion and the C-terminal portion together have a FVIII polypeptide activity. In certain embodiments, the second nucleic acid sequence has at least 85%, at least 90%, at least 95%, at least 96%, at least 97%, at least 98%, or at least 99% sequence identity to nucleotides 1792-4374 of SEQ ID NO: 5. In other embodiments, the second nucleic acid sequence has at least 85%, at least 90%, at least 95%, at least 96%, at least 97%, at least 98%, or at least 99% sequence identity to nucleotides 1792-4374 of SEQ ID NO: 6. In one particular embodiment, the second nucleic acid sequence comprises nucleotides 1792-4374 of SEQ ID NO: 5 or 1792-4374 of SEQ ID NO: 6. In some embodiments, the first nucleic acid sequence linked to the second nucleic acid sequence listed above has at least 60%, at least 70%, at least 80%, at least 90%, at least 95%, at least 96%, at least 97%, at least 98%, or at least 99% sequence identity to nucleotides 58-1791 of SEQ ID NO: 5 or nucleotides 58-1791 of SEQ ID NO: 6. In other embodiments, the first nucleic acid sequence linked to the second nucleic acid sequence listed above has at least 60%, at least 70%, at least 80%, at least 90%, at least 95%, at least 96%, at least 97%, at least 98%, or at least 99% sequence identity to nucleotides 1-1791 of SEQ ID NO: 5 or nucleotides 1-1791 of SEQ ID NO: 6.

In other embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 85%, at least 90%, at least 95%, at least 96%, at least 97%, at least 98%, or at least 99% sequence identity to (i) nucleotides 1792-2277 and 2320-4374 of SEQ ID NO: 5 (i.e., nucleotides 1792-4374 of SEQ ID NO: 5 without the nucleotides encoding the B domain or B domain fragment) or (ii) 1792-2277 and 2320-4374 of SEQ ID NO: 6 (i.e., nucleotides 1792-4374 of SEQ ID NO: 6 without the nucleotides encoding the B domain or B domain fragment); and wherein the N-terminal portion and the C-terminal portion together have a FVIII polypeptide activity. In certain embodiments, the second nucleic acid sequence has at least 85%, at least 90%, at least 95%, at least 96%, at least 97%, at least 98%, or at least 99% sequence identity to nucleotides 1792-2277 and 2320-4374 of SEQ ID NO: 5 (i.e., nucleotides 1792-4374 of SEQ ID NO: 5 without the nucleotides encoding the B domain or B domain fragment). In other embodiments, the second nucleic acid sequence has at least 85%, at least 90%, at least 95%, at least 96%, at least 97%, at least 98%, or at least 99% sequence identity to nucleotides 1792-2277 and 2320-4374 of SEQ ID NO: 6 (i.e., nucleotides 1792-4374 of SEQ ID NO: 6 without the nucleotides encoding the B domain or B domain fragment). In one particular embodiment, the second nucleic acid sequence comprises nucleotides 1792-2277 and 2320-4374 of SEQ ID NO: 5 or 1792-2277 and 2320-4374 of SEQ ID NO: 6 (i.e., nucleotides 1792-4374 of SEQ ID NO: 5 or 1792-4374 of SEQ ID NO: 6 without the nucleotides encoding the B domain or B domain fragment). In some embodiments, the first nucleic acid sequence linked to the second nucleic acid sequence listed above has at least 60%, at least 70%, at least 80%, at least 90%, at least 95%, at least 96%, at least 97%, at least 98%, or at least 99% sequence identity to nucleotides 58-1791 of SEQ ID NO: 5 or nucleotides 58-1791 of SEQ ID NO: 6. In other embodiments, the first nucleic acid sequence linked to the second nucleic acid sequence listed above has at least 60%, at least 70%, at least 80%, at least 90%, at least 95%, at least 96%, at least 97%, at least 98%, or at least 99% sequence identity to nucleotides 1-1791 of SEQ ID NO: 5 or nucleotides 1-1791 of SEQ ID NO: 6.

In some embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least 90%, at least 95%, at least 96%, at least 97%, at least 98%, or at least 99% sequence identity to (i) nucleotides 58-1791 of SEQ ID NO: 1, (ii) nucleotides 58-1791 of SEQ ID NO: 2, (iii) nucleotides 58-1791 of SEQ ID NO: 70, or (iv) nucleotides 58-1791 of SEQ ID NO: 71; and wherein the N-terminal portion and the C-terminal portion together have a FVIII polypeptide activity. In other embodiments, the first nucleotide sequence comprises nucleotides 58-1791 of SEQ ID NO: 1, nucleotides 58-1791 of SEQ ID NO: 2, (iii) nucleotides 58-1791 of SEQ ID NO: 70, or (iv) nucleotides 58-1791 of SEQ ID NO: 71.

In other embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least 90%, at least 95%, at least 96%, at least 97%, at least 98%, or at least 99% sequence identity to (i) nucleotides 1-1791 of SEQ ID NO: 1, (ii) nucleotides 1-1791 of SEQ ID NO: 2, (iii) nucleotides 1-1791 of SEQ ID NO: 70, or (iv) nucleotides 1-1791 of SEQ ID NO: 71; and wherein the N-terminal portion and the C-terminal portion together have a FVIII polypeptide activity. In one embodiment, the first nucleotide sequence comprises nucleotides 1-1791 of SEQ ID NO: 1, nucleotides 1-1791 of SEQ ID NO: 2, (iii) nucleotides 1-1791 of SEQ ID NO: 70, or (iv) nucleotides 1-1791 of SEQ ID NO: 71. In another embodiment, the second nucleotide sequence linked to the first nucleotide sequence has at least 60%, at least 70%, at least 80%, at least 90%, at least 95%, at least 96%, at least 97%, at least 98%, or at least 99% sequence identity to nucleotides 1792-4374 of SEQ ID NO: 1, 1792-4374 of SEQ ID NO: 2, (iii) nucleotides 1792-4374 of SEQ ID NO: 70, or (iv) nucleotides 1792-4374 of SEQ ID NO: 71. In one particular embodiment, the second nucleotide sequence linked to the first nucleotide sequence comprises (i) nucleotides 1792-4374 of SEQ ID NO: 1, (ii) nucleotides 1792-4374 of SEQ ID NO: 2, (iii) nucleotides 1792-4374 of SEQ ID NO: 70, or (iv) nucleotides 1792-4374 of SEQ ID NO: 71. In other embodiments, the second nucleotide sequence linked to the first nucleotide sequence has at least 60%, at least 70%, at least 80%, at least 90%, at least 95%, at least 96%, at least 97%, at least 98%, or at least 99% sequence identity to (i) nucleotides 1792-2277 and 2320-4374 of SEQ ID NO: 1, (ii) nucleotides 1792-2277 and 2320-4374 of SEQ ID NO: 2, (iii) nucleotides 1792-2277 and 2320-4374 of SEQ ID NO: 70, or (iv) nucleotides 1792-2277 and 2320-4374 of SEQ ID NO: 71. In one embodiment, the second nucleotide sequence comprises (i) nucleotides 1792-2277 and 2320-4374 of SEQ ID NO: 1, (ii) nucleotides 1792-2277 and 2320-4374 of SEQ ID NO: 2, (iii) nucleotides 1792-2277 and 2320-4374 of SEQ ID NO: 70, or (iv) nucleotides 1792-2277 and 2320-4374 of SEQ ID NO: 71.

In another embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 90%, at least 95%, at least 96%, at least 97%, at least 98%, or at least 99% sequence identity to (i) nucleotides 1792-4374 of SEQ ID NO: 1, (ii) nucleotides 1792-4374 of SEQ ID NO: 2, (iii) nucleotides 1792-4374 of SEQ ID NO: 70, or (iv) nucleotides 1792-4374 of SEQ ID NO: 71; and wherein the N-terminal portion and the C-terminal portion together have a FVIII polypeptide activity. In one particular embodiment, the second nucleic acid sequence comprises (i) nucleotides 1792-4374 of SEQ ID NO: 1, (ii) nucleotides 1792-4374 of SEQ ID NO: 2, (iii) nucleotides 1792-4374 of SEQ ID NO: 70, or (iv) nucleotides 1792-4374 of SEQ ID NO: 71. In some embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 90%, at least 95%, at least 96%, at least 97%, at least 98%, or at least 99% sequence identity to (i) nucleotides 1792-2277 and 2320-4374 of SEQ ID NO: 1, (ii) nucleotides 1792-2277 and 2320-4374 of SEQ ID NO: 2, (iii) nucleotides 1792-2277 and 2320-4374 of SEQ ID NO: 70, or (iv) nucleotides 1792-2277 and 2320-4374 of SEQ ID NO: 71 (i.e., nucleotides 1792-4374 of SEQ ID NO: 1, nucleotides 1792-4374 of SEQ ID NO: 2, nucleotides 1792-4374 of SEQ ID NO: 70, or nucleotides 1792-4374 of SEQ ID NO: 71 without the nucleotides encoding the B domain or B domain fragment); and wherein the N-terminal portion and the C-terminal portion together have a FVIII polypeptide activity. In one embodiment, the second nucleic acid sequence comprises (i) nucleotides 1792-2277 and 2320-4374 of SEQ ID NO: 1, (ii) nucleotides 1792-2277 and 2320-4374 of SEQ ID NO: 2, (iii) nucleotides 1792-2277 and 2320-4374 of SEQ ID NO: 70, or (iv) nucleotides 1792-2277 and 2320-4374 of SEQ ID NO: 71 (i.e., nucleotides 1792-4374 of SEQ ID NO: 1, nucleotides 1792-4374 of SEQ ID NO: 2, nucleotides 1792-4374 of SEQ ID NO: 70, or nucleotides 1792-4374 of SEQ ID NO: 71 without the nucleotides encoding the B domain or B domain fragment).

In some embodiments, the present disclosure provides an isolated nucleic acid molecule comprising a nucleotide sequence encoding a polypeptide with FVIII activity, wherein the nucleotide sequence comprises a nucleic acid sequence having at least 90%, at least 91%, at least 92%, at least 93%, at least 94%, at least 95%, at least 96%, at least 97%, at least 98%, or at least 99% sequence identity to nucleotides 58 to 4374 of SEQ ID NO: 1. In other embodiments, the nucleotide sequence comprises a nucleic acid sequence having at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 1 (i.e., nucleotides 58-4374 of SEQ ID NO: 1 without the nucleotides encoding the B domain or B domain fragment). In other embodiments, the nucleic acid sequence has at least 90%, at least 91%, at least 92%, at least 93%, at least 94%, at least 95%, at least 96%, at least 97%, at least 98%, or at least 99% sequence identity to SEQ ID NO: 1. In other embodiments, the nucleotide sequence comprises nucleotides 58-2277 and 2320-4374 of SEQ ID NO: 1 (i.e., nucleotides 58-4374 of SEQ ID NO: 1 without the nucleotides encoding the B domain or B domain fragment) or nucleotides 58 to 4374 of SEQ ID NO: 1. In still other embodiments, the nucleotide sequence comprises nucleotides 1-2277 and 2320-4374 of SEQ ID NO: 1 (i.e., nucleotides 1-4374 of SEQ ID NO: 1 without the nucleotides encoding the B domain or B domain fragment) or nucleotides 1 to 4374 of SEQ ID NO: 1.

In some embodiments, the present disclosure provides an isolated nucleic acid molecule comprising a nucleotide sequence encoding a polypeptide with FVIII activity, wherein the nucleotide sequence comprises a nucleic acid sequence having at least 94%, at least 95%, at least 96%, at least 97%, at least 98%, or at least 99% sequence identity to nucleotides 58 to 4374 of SEQ ID NO: 2. In other embodiments, the nucleotide sequence comprises a nucleic acid sequence having at least 94%, at least 95%, at least 96%, at least 97%, at least 98%, or at least 99% sequence identity to nucleotides 58-2277 and 2320-4374 of SEQ ID NO: 2. In other embodiments, the nucleic acid sequence has at least 94%, at least 95%, at least 96%, at least 97%, at least 98%, or at least 99% sequence identity to SEQ ID NO: 2. In other embodiments, the nucleotide sequence comprises nucleotides 58-2277 and 2320-4374 of SEQ ID NO: 2 (i.e., nucleotides 58-4374 of SEQ ID NO: 2 without the nucleotides encoding the B domain or B domain fragment) or nucleotides 58 to 4374 of SEQ ID NO: 2. In still other embodiments, the nucleotide sequence comprises nucleotides 1-2277 and 2320-4374 of SEQ ID NO: 2 (i.e., nucleotides 1-4374 of SEQ ID NO: 2 without the nucleotides encoding the B domain or B domain fragment) or nucleotides 1 to 4374 of SEQ ID NO: 2.

In some embodiments, the present disclosure provides an isolated nucleic acid molecule comprising a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58 to 4374 of SEQ ID NO: 70. In other embodiments, the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58-2277 and 2320-4374 of SEQ ID NO: 70 (i.e., nucleotides 58-4374 of SEQ ID NO: 70 without the nucleotides encoding the B domain or B domain fragment). In other embodiments, the nucleic acid sequence has at least 85%, at least 86%, at least 87%, at least 88%, at least 89%, at least 90%, at least 91%, at least 95%, at least 96%, at least 97%, at least 98%, or at least 99% sequence identity to SEQ ID NO: 70. In other embodiments, the nucleotide sequence comprises nucleotides 58-2277 and 2320-4374 of SEQ ID NO: 70 (i.e., nucleotides 58-4374 of SEQ ID NO: 70 without the nucleotides encoding the B domain or B domain fragment) or nucleotides 58 to 4374 of SEQ ID NO: 70. In still other embodiments, the nucleotide sequence comprises nucleotides 1-2277 and 2320-4374 of SEQ ID NO: 70 (i.e., nucleotides 1-4374 of SEQ ID NO: 70 without the nucleotides encoding the B domain or B domain fragment) or nucleotides 1 to 4374 of SEQ ID NO: 70.

In some embodiments, the present disclosure provides an isolated nucleic acid molecule comprising a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58 to 4374 of SEQ ID NO: 71. In other embodiments, the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58-2277 and 2320-4374 of SEQ ID NO: 71 (i.e., nucleotides 58-4374 of SEQ ID NO: 71 without the nucleotides encoding the B domain or B domain fragment). In other embodiments, the nucleic acid sequence has at least 85%, at least 86%, at least 87%, at least 88%, at least 89%, at least 90%, at least 91%, at least 95%, at least 96%, at least 97%, at least 98%, or at least 99% sequence identity to SEQ ID NO: 71. In other embodiments, the nucleotide sequence comprises nucleotides 58-2277 and 2320-4374 of SEQ ID NO: 71 (i.e., nucleotides 58-4374 of SEQ ID NO: 71 without the nucleotides encoding the B domain or B domain fragment) or nucleotides 58 to 4374 of SEQ ID NO: 71. In still other embodiments, the nucleotide sequence comprises nucleotides 1-2277 and 2320-4374 of SEQ ID NO: 71 (i.e., nucleotides 1-4374 of SEQ ID NO: 71 without the nucleotides encoding the B domain or B domain fragment) or nucleotides 1 to 4374 of SEQ ID NO: 71.

In some embodiments, the present disclosure provides an isolated nucleic acid molecule comprising a nucleotide sequence encoding a polypeptide with FVIII activity, wherein the nucleotide sequence comprises a nucleic acid sequence having at least 92%, at least 93%, at least 94%, at least 95%, at least 96%, at least 97%, at least 98%, or at least 99% sequence identity to nucleotides 58 to 4374 of SEQ ID NO: 3. In other embodiments, the nucleotide sequence comprises a nucleic acid sequence having at least 92%, at least 93%, at least 94%, at least 95%, at least 96%, at least 97%, at least 98%, or at least 99% sequence identity to nucleotides 58-2277 and 2320-4374 of SEQ ID NO: 3 (i.e., nucleotides 58-4374 of SEQ ID NO: 3 without the nucleotides encoding the B domain or B domain fragment). In certain embodiments, the nucleic acid sequence has at least 92%, at least 93%, at least 94%, at least 95%, at least 96%, at least 97%, at least 98%, or at least 99% sequence identity to SEQ ID NO: 3. In some embodiments, the nucleotide sequence comprises nucleotides 58-2277 and 2320-4374 of SEQ ID NO: 3 (i.e., nucleotides 58-4374 of SEQ ID NO: 3 without the nucleotides encoding the B domain or B domain fragment) or nucleotides 58 to 4374 of SEQ ID NO: 3. In still other embodiments, the nucleotide sequence comprises nucleotides 58-2277 and 2320-4374 of SEQ ID NO: 3 (i.e., nucleotides 1-4374 of SEQ ID NO: 3 without the nucleotides encoding the B domain or B domain fragment) or nucleotides 1 to 4374 of SEQ ID NO: 3.

In some embodiments, the present disclosure provides an isolated nucleic acid molecule comprising a nucleotide sequence encoding a polypeptide with FVIII activity, wherein the nucleotide sequence comprises a nucleic acid sequence having at least 91%, at least 92%, at least 93%, at least 94%, at least 95%, at least 96%, at least 97%, at least 98%, or at least 99% sequence identity to nucleotides 58 to 4374 of SEQ ID NO: 4. In other embodiments, the nucleotide sequence comprises a nucleic acid sequence having at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 4 (i.e., nucleotides 58-4374 of SEQ ID NO: 4 without the nucleotides encoding the B domain or B domain fragment). In other embodiments, the nucleic acid sequence has at least 91%, at least 92%, at least 93%, at least 94%, at least 95%, at least 96%, at least 97%, at least 98%, or at least 99% sequence identity to SEQ ID NO: 4. In other embodiments, the nucleotide sequence comprises nucleotides 58-2277 and 2320-4374 of SEQ ID NO: 4 (i.e., nucleotides 58-4374 of SEQ ID NO: 4 without the nucleotides encoding the B domain or B domain fragment) or nucleotides 58 to 4374 of SEQ ID NO: 4. In still other embodiments, the nucleotide sequence comprises nucleotides 1-2277 and 2320-4374 of SEQ ID NO: 4 (i.e., nucleotides 1-4374 of SEQ ID NO: 4 without the nucleotides encoding the B domain or B domain fragment) or nucleotides 1 to 4374 of SEQ ID NO: 4.

In some embodiments, the present disclosure provides an isolated nucleic acid molecule comprising a nucleotide sequence encoding a polypeptide with FVIII activity, wherein the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58 to 4374 of SEQ ID NO: 5. In other embodiments, the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 5 (i.e., nucleotides 58-4374 of SEQ ID NO: 5 without the nucleotides encoding the B domain or B domain fragment). In certain embodiments, the nucleic acid sequence has at least 89%, at least 90%, at least 91%, at least 92%, at least 93%, at least 94%, at least 95%, at least 96%, at least 97%, at least 98%, or at least 99% sequence identity to SEQ ID NO: 5. In some embodiments, the nucleotide sequence comprises nucleotides 58-2277 and 2320-4374 of SEQ ID NO: 5 (i.e., nucleotides 58-4374 of SEQ ID NO: 5 without the nucleotides encoding the B domain or B domain fragment) or nucleotides 58 to 4374 of SEQ ID NO: 5. In still other embodiments, the nucleotide sequence comprises nucleotides 1-2277 and 2320-4374 of SEQ ID NO: 5 (i.e., nucleotides 1-4374 of SEQ ID NO: 5 without the nucleotides encoding the B domain or B domain fragment) or nucleotides 1 to 4374 of SEQ ID NO: 5.

In some embodiments, the present disclosure provides an isolated nucleic acid molecule comprising a nucleotide sequence encoding a polypeptide with FVIII activity, wherein the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58 to 4374 of SEQ ID NO: 6. In other embodiments, the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 6 (i.e., nucleotides 58-4374 of SEQ ID NO: 6 without the nucleotides encoding the B domain or B domain fragment). In certain embodiments, the nucleic acid sequence has at least 89%, at least 90%, at least 91%, at least 92%, at least 93%, at least 94%, at least 95%, at least 96%, at least 97%, at least 98%, or at least 99% sequence identity to SEQ ID NO: 6. In some embodiments, the nucleotide sequence comprises nucleotides 58-2277 and 2320-4374 of SEQ ID NO: 6 (i.e., nucleotides 58-4374 of SEQ ID NO: 6 without the nucleotides encoding the B domain or B domain fragment) or nucleotides 58 to 4374 of SEQ ID NO: 6. In still other embodiments, the nucleotide sequence comprises nucleotides 1-2277 and 2320-4374 of SEQ ID NO: 6 (i.e., nucleotides 1-4374 of SEQ ID NO: 6 without the nucleotides encoding the B domain or B domain fragment) or nucleotides 1 to 4374 of SEQ ID NO: 6.

In some embodiments, the nucleotide sequence comprises a nucleic acid sequence encoding a signal peptide. In certain embodiments, the signal peptide is a FVIII signal peptide. In some embodiments, the nucleic acid sequence encoding a signal peptide is codon optimized. In one particular embodiment, the nucleic acid sequence encoding a signal peptide has at least 60%, at least 70%, at least 80%, at least 90%, at least 95%, at least 96%, at least 97%, at least 98%, at least 99%, or 100% sequence identity to (i) nucleotides 1 to 57 of SEQ ID NO: 1; (ii) nucleotides 1 to 57 of SEQ ID NO: 2; (iii) nucleotides 1 to 57 of SEQ ID NO: 3; (iv) nucleotides 1 to 57 of SEQ ID NO: 4; (v) nucleotides 1 to 57 of SEQ ID NO: 5; (vi) nucleotides 1 to 57 of SEQ ID NO: 6; (vii) nucleotides 1 to 57 of SEQ ID NO: 70; (viii) nucleotides 1 to 57 of SEQ ID NO: 71; or (ix) nucleotides 1 to 57 of SEQ ID NO: 68.

SEQ ID NOs: 1-6, 70, and 71 are optimized versions of SEQ ID NO: 16, the starting or “parental” or “wild-type” FVIII nucleotide sequence. SEQ ID NO: 16 encodes a B domain-deleted human FVIII. While SEQ ID NOs: 1-6, 70, and 71 are derived from a specific B domain-deleted form of FVIII (SEQ ID NO: 16), it is to be understood that the present disclosure is also directed to optimized versions of nucleic acids encoding other versions of FVIII. For example, other versions of FVIII can include full length FVIII, other B-domain deletions of FVIII (described below), or other fragments of FVIII that retain FVIII activity.

“A polypeptide with FVIII activity” as used herein means a functional FVIII polypeptide in its normal role in coagulation, unless otherwise specified. The term a polypeptide with FVIII activity includes a functional fragment, variant, analog, or derivative thereof that retains the function of full-length wild-type Factor VIII in the coagulation pathway. “A polypeptide with FVIII activity” is used interchangeably with FVIII protein, FVIII polypeptide, or FVIII. Examples of FVIII functions include, but are not limited to, an ability to activate coagulation, an ability to act as a cofactor for factor IX, or an ability to form a tenase complex with factor IX in the presence of Ca²⁺ and phospholipids, which then converts Factor X to the activated form Xa. In one embodiment, a polypeptide having FVIII activity comprises two polypeptide chains, the first chain having the FVIII heavy chain and the second chain having the FVIII light chain. In another embodiment, the polypeptide having FVIII activity is single chain FVIII. Single chain FVIII can contain one or more mutation or substitutions at amino acid residue 1645 and/or 1648 corresponding to mature FVIII sequence. See International Application No. PCT/US2012/045784, incorporated herein by reference in its entirety. The FVIII protein can be the human, porcine, canine, rat, or murine FVIII protein. In addition, comparisons between FVIII from humans and other species have identified conserved residues that are likely to be required for function (Cameron et al., Thromb. Haemost. 79:317-22 (1998); U.S. Pat. No. 6,251,632).

The “B domain” of FVIII, as used herein, is the same as the B domain known in the art that is defined by internal amino acid sequence identity and sites of proteolytic cleavage by thrombin, e.g., residues Ser741-Arg1648 of full length human FVIII. The other human FVIII domains are defined by the following amino acid residues: A1, residues Ala1-Arg372; A2, residues Ser373-Arg740; A3, residues Ser1690-Ile2032; C1, residues Arg2033-Asn2172; C2, residues Ser2173-Tyr2332. The A3-C1-C2 sequence includes residues Ser1690-Tyr2332. The remaining sequence, residues Glu1649-Arg1689, is usually referred to as the FVIII light chain activation peptide. The locations of the boundaries for all of the domains, including the B domains, for porcine, mouse and canine FVIII are also known in the art. An example of a BDD FVIII is REFACTO® recombinant BDD FVIII (Wyeth Pharmaceuticals, Inc.).

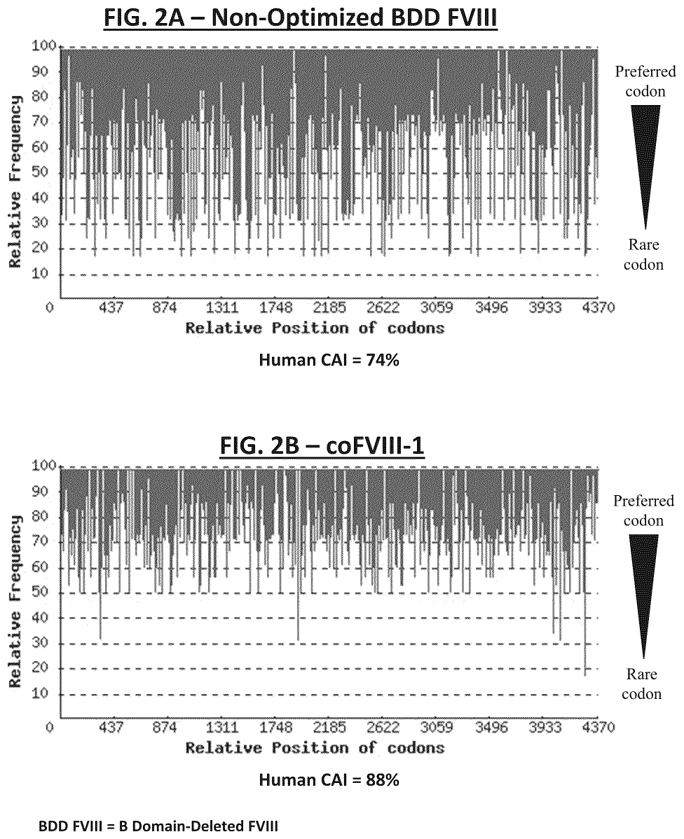

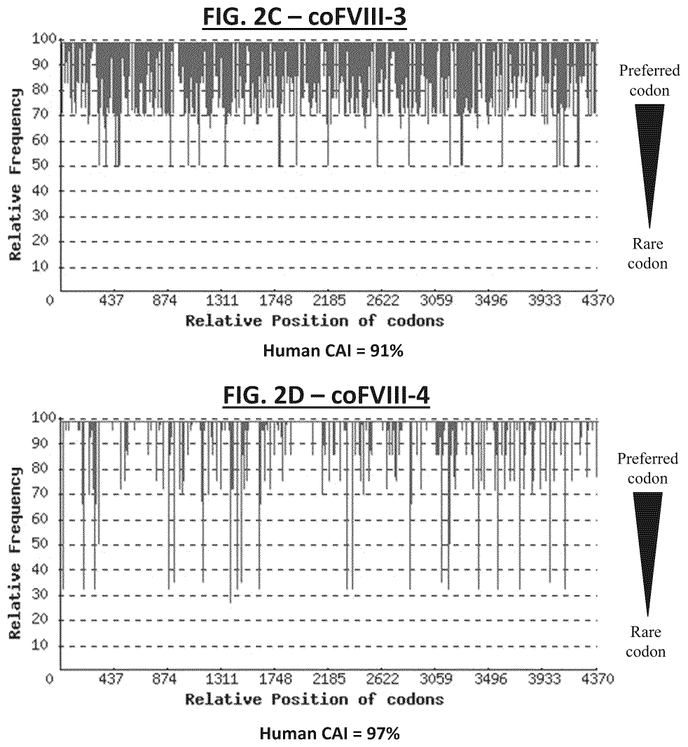

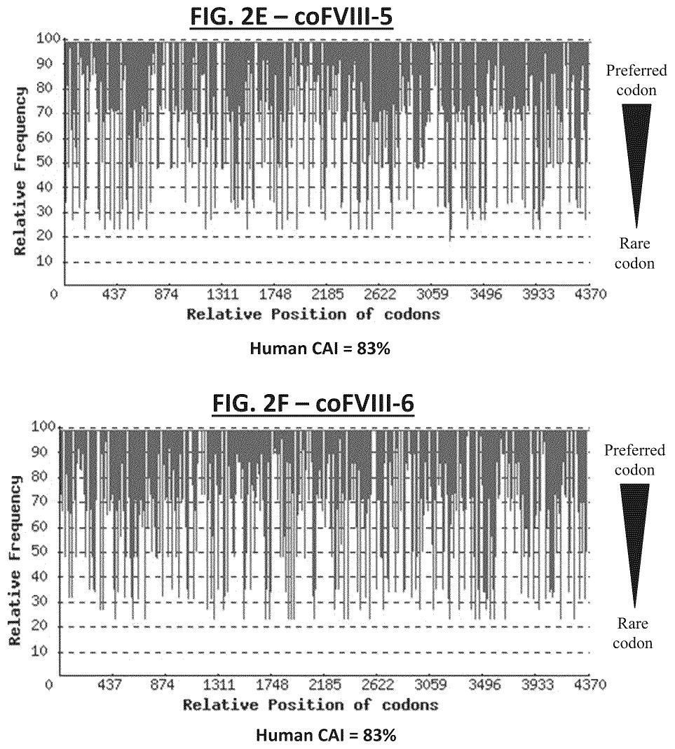

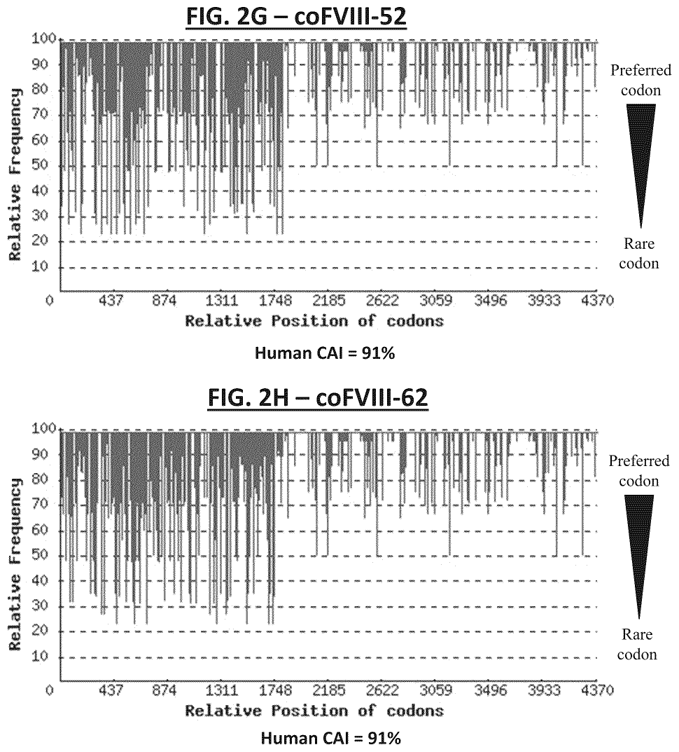

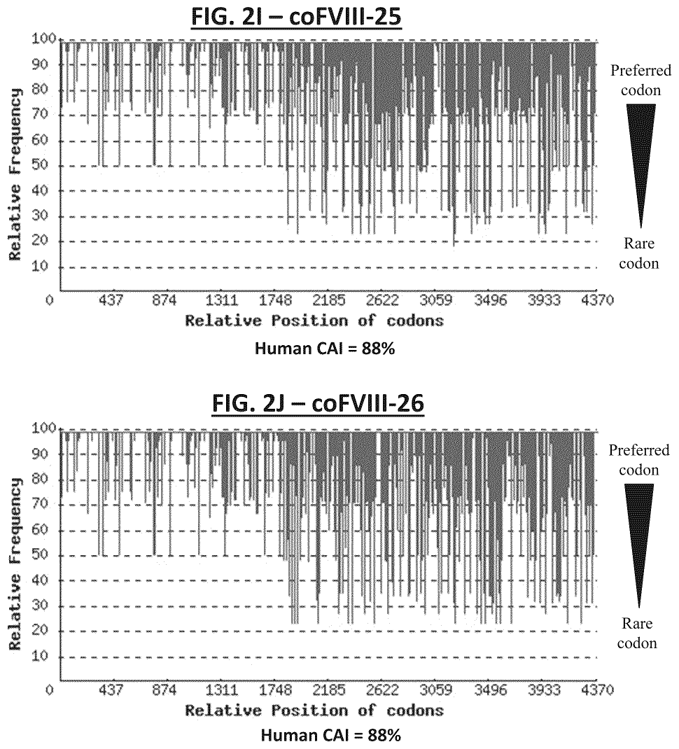

A “B domain deleted FVIII” can have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, and 6,458,563, each of which is incorporated herein by reference in its entirety. In some embodiments, a B domain deleted FVIII sequence of the present disclosure comprises any one of the deletions disclosed at col. 4, line 4 to col. 5, line 28 and examples 1-5 of U.S. Pat. No. 6,316,226 (also in U.S. Pat. No. 6,346,513). In some embodiments, a B domain deleted FVIII of the present disclosure has a deletion disclosed at col. 2, lines 26-51 and examples 5-8 of U.S. Pat. No. 5,789,203 (also U.S. Pat. Nos. 6,060,447, 5,595,886, and 6,228,620). In some embodiments, a B domain deleted FVIII has a deletion described in col. 1, lines 25 to col. 2, line 40 of U.S. Pat. No. 5,972,885; col. 6, lines 1-22 and example 1 of U.S. Pat. No. 6,048,720; col. 2, lines 17-46 of U.S. Pat. No. 5,543,502; col. 4, line 22 to col. 5, line 36 of U.S. Pat. No. 5,171,844; col. 2, lines 55-68, FIG. 2, and example 1 of U.S. Pat. No. 5,112,950; col. 2, line 2 to col. 19, line 21 and table 2 of U.S. Pat. No. 4,868,112; col. 2, line 1 to col. 3, line 19, col. 3, line 40 to col. 4, line 67, col. 7, line 43 to col. 8, line 26, and col. 11, line 5 to col. 13, line 39 of U.S. Pat. No. 7,041,635; or col. 4, lines 25-53, of U.S. Pat. No. 6,458,563. In some embodiments, a B domain deleted FVIII has a deletion of most of the B domain, but still contains amino-terminal sequences of the B domain that are essential for in vivo proteolytic processing of the primary translation product into two polypeptide chain, as disclosed in WO 91/09122, which is incorporated herein by reference in its entirety. In some embodiments, a B domain deleted FVIII is constructed with a deletion of amino acids 747-1638, i.e., virtually a complete deletion of the B domain. Hoeben R. C., et al. J. Biol. Chem. 265 (13): 7318-7323 (1990), incorporated herein by reference in its entirety. A B domain deleted FVIII can also contain a deletion of amino acids 771-1666 or amino acids 868-1562 of FVIII. Meulien P., et al. Protein Eng. 2(4): 301-6 (1988), incorporated herein by reference in its entirety. Additional B domain deletions that are part of the disclosure include, e.g.: deletion of amino acids 982 through 1562 or 760 through 1639 (Toole et al., Proc. Natl. Acad. Sci. U.S.A. (1986) 83, 5939-5942)), 797 through 1562 (Eaton, et al. Biochemistry (1986) 25:8343-8347)), 741 through 1646 (Kaufman (PCT published application No. WO 87/04187)), 747-1560 (Sarver, et al., DNA (1987) 6:553-564)), 741 through 1648 (Pasek (PCT application No. 88/00831)), 816 through 1598 or 741 through 1689 (Lagner (Behring Inst. Mitt. (1988) No 82:16-25, EP 295597)), each of which is incorporated herein by reference in its entirety. Each of the foregoing deletions can be made in any FVIII sequence.

A number of functional FVIII molecules, including B-domain deletions, are disclosed in the following U.S. Pat. Nos. 6,316,226 and 6,346,513, both assigned to Baxter; U.S. Pat. No. 7,041,635 assigned to In2Gen; U.S. Pat. Nos. 5,789,203, 6,060,447, 5,595,886, and 6,228,620 assigned to Chiron; U.S. Pat. Nos. 5,972,885 and 6,048,720 assigned to Biovitrum, U.S. Pat. Nos. 5,543,502 and 5,610,278 assigned to Novo Nordisk; U.S. Pat. No. 5,171,844 assigned to Immuno Ag; U.S. Pat. No. 5,112,950 assigned to Transgene S. A.; U.S. Pat. No. 4,868,112 assigned to Genetics Institute, each of which is incorporated herein by reference in its entirety.

### Codon Optimization

In one embodiment, the present disclosure provides an isolated nucleic acid molecule comprising a nucleotide sequence that encodes a polypeptide with FVIII activity, wherein the nucleic acid sequence has been codon optimized. In another embodiment, the starting nucleic acid sequence that encodes a polypeptide with FVIII activity and that is subject to codon optimization is SEQ ID NO: 16. In some embodiments, the sequence that encodes a polypeptide with FVIII activity is codon optimized for human expression. In other embodiments, the sequence that encodes a polypeptide with FVIII activity is codon optimized for murine expression. SEQ ID NOs: 1-6, 70, and 71 are codon optimized versions of SEQ ID NO: 16, optimized for human expression.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism.

Deviations in the nucleotide sequence that comprises the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 1. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

TABLE 1
The Standard Genetic Code

| | T | C | A | G |
|---|---|---|---|---|
| T | TTT Phe (F) | TCT Ser (S) | TAT Tyr (Y) | TGT Cys (C) |
| | TTC″ | TCC″ | TAC″ | TGC |
| | TTA Leu (L) | TCA″ | TAA Stop | TGA Stop |
| | TTG″ | TCG″ | TAG Stop | TGG Trp (W) |
| C | CTT Leu (L) | CCT Pro (P) | CAT His (H) | CGT Arg (R) |
| | CTC″ | CCC″ | CAC″ | CGC″ |
| | CTA″ | CCA″ | CAA Gln (Q) | CGA″ |
| | CTG″ | CCG″ | CAG″ | CGG″ |
| A | ATT Ile (I) | ACT Thr (T) | AAT Asn (N) | AGT Ser (S) |
| | ATC″ | ACC″ | AAC″ | AGC″ |
| | ATA″ | ACA″ | AAA Lys (K) | AGA Arg (R) |
| | ATG Met (M) | ACG″ | AAG″ | AGG″ |
| G | GTT Val (V) | GCT Ala (A) | GAT Asp (D) | GGT Gly (G) |
| | GTC″ | GCC″ | GAC″ | GGC″ |
| | GTA″ | GCA″ | GAA Glu (E) | GGA″ |
| | GTG″ | GCG″ | GAG″ | GGG″ |

Many organisms display a bias for use of particular codons to code for insertion of a particular amino acid in a growing peptide chain. Codon preference, or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, the relative frequencies of codon usage have been calculated. Codon usage tables are available, for example, at the “Codon Usage Database” available at www.kazusa.or.jp/codon/(visited Jun. 18, 2012). See Nakamura, Y., et al. Nucl. Acids Res. 28:292 (2000).

Randomly assigning codons at an optimized frequency to encode a given polypeptide sequence can be done manually by calculating codon frequencies for each amino acid, and then assigning the codons to the polypeptide sequence randomly. Additionally, various algorithms and computer software programs can be used to calculate an optimal sequence.

In some embodiments, the nucleic acid molecule comprises one or more properties: (a) the nucleic acid molecule or a portion thereof has an increased the human codon adaptation index relative to SEQ ID NO: 16; (b) the nucleotide sequence or a portion thereof has an increased frequency of optimal codons relative to SEQ ID NO:16; (c) the nucleotide sequence or a portion thereof contains a higher percentage of G/C nucleotides compared to the percentage of G/C nucleotides in SEQ ID NO: 16; (d) the nucleotide sequence or a portion thereof has an increased relative synonymous codon usage relative to SEQ ID NO: 16; (e) the nucleotide sequence or a portion thereof is a reduced effective number of codons relative SEQ ID NO: 16; (f) the nucleotide sequence contains fewer MARS/ARS sequences (SEQ ID NOs: 21 and 22) relative to SEQ ID NO: 16; (g) the nucleotide sequence contains fewer destabilizing elements (SEQ ID NOs: 23 and 24) relative to SEQ ID NO: 16; (i) the nucleotide sequence does not contain a poly-T sequence, (j) the nucleotide sequence does not contain a poly-A sequence; or (k) any combination thereof. In some embodiments, the nucleic acid molecules contains at least two, at least three, at least four, at least five, at least six, at least seven, at least eight, at least nine, or ten characteristics of (a) to (j).

### Codon Adaptation Index

In one embodiment, the present disclosure provides an isolated nucleic acid molecule comprising a nucleotide sequence described herein that encodes a polypeptide with FVIII activity, wherein the human codon adaptation index is increased relative to SEQ ID NO: 16. For example, the nucleotide sequence can have a human codon adaptation index that is at least about 0.75 (75%), at least about 0.76 (76%), at least about 0.77 (77%), at least about 0.78 (78%), at least about 0.79 (79%), at least about 0.80 (80%), at least about 0.81 (81%), at least about 0.82 (82%), at least about 0.83 (83%), at least about 0.84 (84%), at least about 0.85 (85%), at least about 0.86 (86%), at least about 0.87 (87%), at least about 0.88 (88%), at least about 0.89 (89%), at least about 0.90 (90%), at least about 0.91 (91%), at least about 0.92 (92%), at least about 0.93 (93%), at least about 0.94 (94%), at least about 0.95 (95%), at least about 0.96 (96%), at least about 0.97 (97%), at least about 0.98 (98%), or at least about 0.99 (99%). In some embodiments, the nucleotide sequence has a human codon adaptation index that is at least about 0.88 (88%). In other embodiments, the nucleotide sequence has a human codon adaptation index that is at least about 0.91 (91%). In other embodiments, the nucleotide sequence has a human codon adaptation index that is at least about 0.91 (97%).

In one particular embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the human codon adaptation index of the nucleotide sequence is increased relative to SEQ ID NO: 16. In some embodiments, the nucleotide sequence has a human codon adaptation index that is at least about 0.75 (75%), at least about 0.76 (76%), at least about 0.77 (77%), at least about 0.78 (78%), at least about 0.79 (79%), at least about 0.80 (80%), at least about 0.81 (81%), at least about 0.82 (82%), at least about 0.83 (83%), at least about 0.84 (84%), at least about 0.85 (85%), at least about 0.86 (86%), at least about 0.87 (87%), at least about 0.88 (88%), at least about 0.89 (89%), at least about 0.90 (90%), or at least about 0.91 (91%). In one particular the nucleotide sequence has a human codon adaptation index that is at least about 0.88 (88%). In another embodiment, the nucleotide sequence has a human codon adaptation index that is at least about 0.91 (91%).

In another embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-2277 and 2320-4374 of SEQ ID NO: 5 or (ii) 1792-2277 and 2320-4374 of SEQ ID NO: 6; wherein the N-terminal portion and the C-terminal portion together have a FVIII polypeptide activity; and wherein the human codon adaptation index of the nucleotide sequence is increased relative to SEQ ID NO: 16. In some embodiments, the nucleotide sequence has a human codon adaptation index that is at least about 0.75 (75%), at least about 0.76 (76%), at least about 0.77 (77%), at least about 0.78 (78%), at least about 0.79 (79%), at least about 0.80 (80%), at least about 0.81 (81%), at least about 0.82 (82%), at least about 0.83 (83%), at least about 0.84 (84%), at least about 0.85 (85%), at least about 0.86 (86%), at least about 0.87 (87%), or at least about 0.88 (88%). In one particular the nucleotide sequence has a human codon adaptation index that is at least about 0.83 (83%). In another embodiment, the nucleotide sequence has a human codon adaptation index that is at least about 0.88 (88%).

In other embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the human codon adaptation index of the nucleotide sequence is increased relative to SEQ ID NO: 16. In some embodiments, the nucleotide sequence has a human codon adaptation index that is at least about 0.75 (75%), at least about 0.76 (76%), at least about 0.77 (77%), at least about 0.78 (78%), at least about 0.79 (79%), at least about 0.80 (80%), at least about 0.81 (81%), at least about 0.82 (82%), at least about 0.83 (83%), at least about 0.84 (84%), at least about 0.85 (85%), at least about 0.86 (86%), at least about 0.87 (87%), or at least about 0.88 (88%). In one particular the nucleotide sequence has a human codon adaptation index that is at least about 0.75 (75%). In another embodiment, the nucleotide sequence has a human codon adaptation index that is at least about 0.83 (83%). In another embodiment, the nucleotide sequence has a human codon adaptation index that is at least about 0.88 (88%). In another embodiment, the nucleotide sequence has a human codon adaptation index that is at least about 0.91 (91%). In another embodiment, the nucleotide sequence has a human codon adaptation index that is at least about 0.97 (97%).

In some embodiments, the isolated nucleic acid molecule of the present disclosure has an increased frequency of optimal codons (FOP) relative to SEQ ID NO: 16. In certain embodiments, the FOP of the isolated nucleic acid molecule is at least about 40, at least about 45, at least about 50, at least about 55, at least about 60, at least about 64, at least about 65, at least about 70, at least about 75, at least about 79, at least about 80, at least about 85, or at least about 90.

In other embodiments, the isolated nucleic acid molecule of the present disclosure has an increased relative synonymous codon usage (RCSU) relative to SEQ ID NO: 16. In some embodiments, the RCSU of the isolated nucleic acid molecule is greater than 1.5. In other embodiments, the RCSU of the isolated nucleic acid molecule is greater than 2.0. In certain embodiments, the RCSU of the isolated nucleic acid molecule is at least about 1.5, at least about 1.6, at least about 1.7, at least about 1.8, at least about 1.9, at least about 2.0, at least about 2.1, at least about 2.2, at least about 2.3, at least about 2.4, at least about 2.5, at least about 2.6, or at least about 2.7.

In still other embodiments, the isolated nucleic acid molecule of the present disclosure has a decreased effective number of codons relative to SEQ ID NO: 16. In some embodiments, the isolated nucleic acid molecule has an effective number of codons of less than about 50, less than about 45, less than about 40, less than about 35, less than about 30, or less than about 25. In one particular embodiment, the isolated nucleic acid molecule has an effective number of codons of about 40, about 35, about 30, about 25, or about 20.

### G/C Content Optimization

In some embodiments, the present disclosure provides an isolated nucleic acid molecule comprising a nucleotide sequence described herein that encodes a polypeptide with FVIII activity, wherein the nucleotide sequence contains a higher percentage of G/C nucleotides compared to the percentage of G/C nucleotides in SEQ ID NO: 16. In other embodiments, the nucleotide sequence that encodes a polypeptide with FVIII activity has a G/C content that is at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, or at least about 60%.

In one particular embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence contains a higher percentage of G/C nucleotides compared to the percentage of G/C nucleotides in SEQ ID NO: 16. In some embodiments, the nucleotide sequence that encodes a polypeptide with FVIII activity has a G/C content that is at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, or at least about 58%. In one particular embodiment, the nucleotide sequence that encodes a polypeptide with FVIII activity has a G/C content that is at least about 58%.

In another embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment), or (iv) 1792-2277 and 2320-4374 of SEQ ID NO: 6 (i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the nucleotide sequence contains a higher percentage of G/C nucleotides compared to the percentage of G/C nucleotides in SEQ ID NO: 16. In other embodiments, the nucleotide sequence that encodes a polypeptide with FVIII activity has a G/C content that is at least about 45%, at least about 46%, at least about 47%, at least about 48%, at least about 49%, at least about 50%, at least about 51%, at least about 52%, at least about 53%, at least about 54%, at least about 55%, at least about 56%, or at least about 57%. In one particular embodiment, the nucleotide sequence that encodes a polypeptide with FVIII activity has a G/C content that is at least about 52%. In another embodiment, the nucleotide sequence that encodes a polypeptide with FVIII activity has a G/C content that is at least about 55%. In another embodiment, the nucleotide sequence that encodes a polypeptide with FVIII activity has a G/C content that is at least about 57%.

In other embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the nucleotide sequence contains a higher percentage of G/C nucleotides compared to the percentage of G/C nucleotides in SEQ ID NO: 16. In other embodiments, the nucleotide sequence that encodes a polypeptide with FVIII activity has a G/C content that is at least about 45%. In one particular embodiment, the nucleotide sequence that encodes a polypeptide with FVIII activity has a G/C content that is at least about 52%. In another embodiment, the nucleotide sequence that encodes a polypeptide with FVIII activity has a G/C content that is at least about 55%. In another embodiment, the nucleotide sequence that encodes a polypeptide with FVIII activity has a G/C content that is at least about 57%. In another embodiment, the nucleotide sequence that encodes a polypeptide with FVIII activity has a G/C content that is at least about 58%. In still another embodiment, the nucleotide sequence that encodes a polypeptide with FVIII activity has a G/C content that is at least about 60%.

“G/C content” (or guanine-cytosine content), or “percentage of G/C nucleotides,” refers to the percentage of nitrogenous bases in a DNA molecule that are either guanine or cytosine. G/C content can be calculated using the following formula:

$$\frac{G + C}{A + T + G + C} \times 100 \qquad ( III )$$

Human genes are highly heterogeneous in their G/C content, with some genes having a G/C content as low as 20%, and other genes having a G/C content as high as 95%. In general, G/C rich genes are more highly expressed. In fact, it has been demonstrated that increasing the G/C content of a gene can lead to increased expression of the gene, due mostly to an increase in transcription and higher steady state mRNA levels. See Kudla et al., PLoS Biol., 4(6): e180 (2006).

### Matrix Attachment Region-Like Sequences

In some embodiments, the present disclosure provides an isolated nucleic acid molecule comprising a nucleotide sequence described herein that encodes a polypeptide with FVIII activity, wherein the nucleotide sequence contains fewer MARS/ARS sequences relative to SEQ ID NO: 16. In other embodiments, the nucleotide sequence that encodes a polypeptide with FVIII activity contains at most 6, at most 5, at most 4, at most 3, or at most 2 MARS/ARS sequences. In other embodiments, the nucleotide sequence that encodes a polypeptide with FVIII activity contains at most 1 MARS/ARS sequence. In yet other embodiments, the nucleotide sequence that encodes a polypeptide with FVIII activity does not contain a MARS/ARS sequence.

In one particular embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence contains fewer MARS/ARS sequences relative to SEQ ID NO: 16. In other embodiments, the nucleotide sequence that encodes a polypeptide with FVIII activity contains at most 6, at most 5, at most 4, at most 3, or at most 2 MARS/ARS sequences. In other embodiments, the nucleotide sequence that encodes a polypeptide with FVIII activity contains at most 1 MARS/ARS sequence. In yet other embodiments, the nucleotide sequence that encodes a polypeptide with FVIII activity does not contain a MARS/ARS sequence.

In another embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment); or (iv) nucleotides 1792-2277 and 2320-4374 of SEQ ID NO: 6 (i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the nucleotide sequence contains fewer MARS/ARS sequences relative to SEQ ID NO: 16. In other embodiments, the nucleotide sequence that encodes a polypeptide with FVIII activity contains at most 6, at most 5, at most 4, at most 3, or at most 2 MARS/ARS sequences. In other embodiments, the nucleotide sequence that encodes a polypeptide with FVIII activity contains at most 1 MARS/ARS sequence. In yet other embodiments, the nucleotide sequence that encodes a polypeptide with FVIII activity does not contain a MARS/ARS sequence.

In other embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of SEQ ID NOs: 1, 2, 3, 4, 5, 6, 70, or 71 or (ii) nucleotides 58-2277 and 2320-4374 of SEQ ID NOs: 1, 2, 3, 4, 5, 6, 70, or 71 (i.e., nucleotides 58-4374 of SEQ ID NO: 1, 2, 3, 4, 5, 6, 70, or 71 without the nucleotides encoding the B domain or B domain fragment); and wherein the nucleotide sequence contains fewer MARS/ARS sequences relative to SEQ ID NO: 16. In other embodiments, the nucleotide sequence that encodes a polypeptide with FVIII activity contains at most 6, at most 5, at most 4, at most 3, or at most 2 MARS/ARS sequences. In other embodiments, the nucleotide sequence that encodes a polypeptide with FVIII activity contains at most 1 MARS/ARS sequence. In yet other embodiments, the nucleotide sequence that encodes a polypeptide with FVIII activity does not contain a MARS/ARS sequence.

AT-rich elements in the human FVIII nucleotide sequence that share sequence similarity with Saccharomyces cerevisiae autonomously replicating sequences (ARSs) and nuclear-matrix attachment regions (MARs) have been identified. (Fallux et al., Mol. Cell. Biol. 16:4264-4272 (1996). One of these elements has been demonstrated to bind nuclear factors in vitro and to repress the expression of a chloramphenicol acetyltransferase (CAT) reporter gene. Id. It has been hypothesized that these sequences can contribute to the transcriptional repression of the human FVIII gene. Thus, in one embodiment, all MAR/ARS sequences are abolished in the FVIII gene of the present disclosure. There are four MAR/ARS ATATTT sequences (SEQ ID NO: 21) and three MAR/ARS AAATAT sequences (SEQ ID NO: 22) in the parental FVIII sequence (SEQ ID NO: 16). All of these sites were mutated to destroy the MAR/ARS sequences in the optimized FVIII sequences (SEQ ID NOs: 1-6). The location of each of these elements, and the sequence of the corresponding nucleotides in the optimized sequences are shown in Table 2, below.

TABLE 2
Summary of Changes to Repressive Elements

| | Starting | | | | | | | | |
|---|---|---|---|---|---|---|---|---|---|
| | BDD | | | | | | | | |
| | FVIII | | | | | | | | |
| Location | Sequence | Optimized BDD FVIII Sequence | | | | | | | |
| of | (SEQ ID | SEQ ID | SEQ ID | SEQ ID | SEQ ID | SEQ ID | SEQ ID | SEQ ID | SEQ ID |
| Element | NO: 16) | NO: 1 | NO: 2 | NO: 3 | NO: 4 | NO: 5 | NO: 6 | NO: 70 | NO: 71 |

Destabilizing Sequences

| | | | | | | | | | |
|---|---|---|---|---|---|---|---|---|---|
| 639 | ATTTA | GTTTA | GTTCA | GTTCA | GTTCA | GTTCA | GTTCA | GTTCA | GTTCA |
| 1338 | ATTTA | GTTTA | GTTCA | CTTCA | GTTCA | GTTCA | GTTCA | CTTCA | GTTCA |
| 1449 | ATTTA | CITTA | CTTCA | CTTCA | CTTCA | CTTCA | CTTCA | CTTCA | CTTCA |
| 1590 | TAAAT | TAAAT | CAAGT | CAAGT | TAAGT | CAAGT | CAAGT | CAAGT | TAAGT |
| 1623 | TAAAT | CAAAA | GAAGA | CTAAG | CAAGA | CAAGA | CAAGA | TAAGT | CAAGA |
| 2410 | ATTTA | ATCTA | ATCTA | ATCTA | ATCTA | ATCTA | ATCTA | ATCTA | ATCTA |
| 2586 | ATTTA | GTTTA | GTTCA | GTTCA | GTTCA | GTTCA | GTTCA | GTTCA | GTTCA |
| 2630 | TAAAT | TGAAT | TGAAC | TGAAC | TGAAC | TCAAT | TGAAC | TCAAT | TGAAC |
| 3884 | ATTTA | ATCTG | ACCTG | ACCTG | ACCTG | ATCTG | ACCTG | ATCTG | ACCTG |
| 3887 | TAAAT | TGAAC | TGAAC | TGAAC | TGAAC | TGAAC | TGAAC | TGAAC | TGAAC |

Potential Promoter Binding Sites

| | | | | | | | | | |
|---|---|---|---|---|---|---|---|---|---|
| 641 | TTATA | TTATC | TCATC | TCATT | TCATC | TCATC | TCATC | TCATT | TCATC |
| 1275 | TATAA | CTATA | TTACA | CTACA | GTACA | CTACA | CTACA | CTACA | GTACA |
| 1276 | TTATA | TATAA | TACAA | TACAA | TACAA | TACAA | TACAA | TACAA | TACAA |
| 1445 | TTATA | TCATC | TCATC | TTATC | TCATC | TCATC | TCATC | TTATC | TCATC |
| 1474 | TATAA | TATAA | TACAA | TACAA | TACAA | TACAA | TACAA | TACAA | TACAA |
| 1588 | TATAA | TATAA | TACAA | TACAA | TATAA | TACAA | TACAA | TACAA | TATAA |
| 2614 | TTATA | CTGTA | CTGTA | CTGTA | CTGTA | TTGTA | CTGTA | TTGTA | CTGTA |
| 2661 | TATAA | CATCA | CATCA | CATCA | CATCA | CATCA | CATCC | CATCA | CATCC |
| 3286 | TATAA | TATAA | TACAA | TACAA | TACAA | TACAA | TACAA | TACAA | TACAA |
| 3840 | TTATA | TTATA | TTACT | CTACA | CTACA | CTACA | CTACT | CTACA | CTACT |

Matrix Attachment-Like Sequences (MARS/ARS)

| | | | | | | | | | |
|---|---|---|---|---|---|---|---|---|---|
| 1287 | ATATTT | GTATCT | GTACCT | GTACCT | GTATCT | GTACCT | GTACCT | GTACCT | GTATCT |
| 1447 | ATATTT | ATCTTT | ATCTTC | ATCTTC | ATCTTC | ATCTTC | ATCTTC | ATCTTC | ATCTTC |
| 1577 | AAATAT | AAATCT | AGATCT | AAATCT | AAATCT | AGATCT | AGATCT | AAATCT | AAATCT |
| 1585 | AAATAT | AAGTAT | AAGTAC | AAGTAC | AAGTAT | AAGTAC | AAGTAC | AAGTAC | AAGTAT |
| 2231 | ATATTT | ACATCA | ATATCA | ACATCA | ACATCA | ACATCT | ATATCT | ACATCT | ATATCT |
| 3054 | AAATAT | AAACAT | GAATAT | GAACAT | GAACAT | GAACAT | GAATAT | GAACAT | GAATAT |
| 3788 | ATATTT | ATATCT | ATATCT | ACATCT | ACATCT | ACATCT | ACATCT | ACATCT | ACATCT |

AU Rich Sequence Elements (AREs)

| | | | | | | | | | |
|---|---|---|---|---|---|---|---|---|---|
| 2468 | 1. ATTTT | ACTTCATC | ACTTCATC | ACTTCATT | ACTTCATT | ACTTTATT | ACTTTATC | ACTTTATT | ACTTTATC |
| | ATT | | | | | | | | |
| 3790 | 2. ATTTT | ATCTTTAA | ATCTTCAA | ATCTTCAA | ATCTTCAA | ATCTTCAA | ATCTTCAA | ATCTTCAA | ATCTTCAA |
| | TAA | | | | | | | | |

Poly A/Poly T Sequences

| | | | | | | | | | |
|---|---|---|---|---|---|---|---|---|---|
| 3273 | AAAAAAA | GAAAAAA | GAAGAAG | GAAGAAG | GAAGAAG | GAAGAAG | CAAGAAG | GAAGAAG | CAAGAAG |
| 4195 | TTTTTT | TTCTTT | TTCTTC | TTCTTC | TTCTTC | TTCTTC | TTCTTC | TTCTTCC | TTCTTCC |

Splice Sites

| | | | | | | | | | |
|---|---|---|---|---|---|---|---|---|---|
| 2203 | GGTGAT | GGGGAC | GGCGAC | GGGGAC | GGGGAC | GGAGAC | GGAGAC | GGAGAC | GGAGAC |

### Destabilizing Sequences

In some embodiments, the present disclosure provides an isolated nucleic acid molecule comprising a nucleotide sequence described herein that encodes a polypeptide with FVIII activity, wherein the nucleotide sequence contains fewer destabilizing elements relative to SEQ ID NO: 16. In other embodiments, the nucleotide sequence that encodes a polypeptide with FVIII activity contains at most 9, at most 8, at most 7, at most 6, or at most 5 destabilizing elements. In other embodiments, the nucleotide sequence that encodes a polypeptide with FVIII activity contains at most 4, at most 3, at most 2, or at most 1 destabilizing elements. In yet other embodiments, the nucleotide sequence that encodes a polypeptide with FVIII activity does not contain a destabilizing element.

In one particular embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence contains fewer destabilizing elements relative to SEQ ID NO: 16. In other embodiments, the nucleotide sequence that encodes a polypeptide with FVIII activity contains at most 9, at most 8, at most 7, at most 6, or at most 5 destabilizing elements. In other embodiments, the nucleotide sequence that encodes a polypeptide with FVIII activity contains at most 4, at most 3, at most 2, or at most 1 destabilizing elements. In yet other embodiments, the nucleotide sequence that encodes a polypeptide with FVIII activity does not contain a destabilizing element.

In another embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment); or (iv) nucleotides 1792-2277 and 2320-4374 of SEQ ID NO: 6 (i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the nucleotide sequence contains fewer destabilizing elements relative to SEQ ID NO: 16. In other embodiments, the nucleotide sequence that encodes a polypeptide with FVIII activity contains at most 9, at most 8, at most 7, at most 6, or at most 5 destabilizing elements. In other embodiments, the nucleotide sequence that encodes a polypeptide with FVIII activity contains at most 4, at most 3, at most 2, or at most 1 destabilizing elements. In yet other embodiments, the nucleotide sequence that encodes a polypeptide with FVIII activity does not contain a destabilizing element.

In other embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the nucleotide sequence contains fewer destabilizing elements relative to SEQ ID NO: 16. In other embodiments, the nucleotide sequence that encodes a polypeptide with FVIII activity contains at most 9, at most 8, at most 7, at most 6, or at most 5 destabilizing elements. In other embodiments, the nucleotide sequence that encodes a polypeptide with FVIII activity contains at most 4, at most 3, at most 2, or at most 1 destabilizing elements. In yet other embodiments, the nucleotide sequence that encodes a polypeptide with FVIII activity does not contain a destabilizing element.

There are ten destabilizing elements in the parental FVIII sequence (SEQ ID NO: 16): six ATTTA sequences (SEQ ID NO: 23) and four TAAAT sequences (SEQ ID NO: 24). In one embodiment, sequences of these sites were mutated to destroy the destabilizing elements in optimized FVIII SEQ ID NOs: 1-6, 70, and 71. The location of each of these elements, and the sequence of the corresponding nucleotides in the optimized sequences are shown in Table 2.

### Potential Promoter Binding Sites

In some embodiments, the present disclosure provides an isolated nucleic acid molecule comprising a nucleotide sequence described herein that encodes a polypeptide with FVIII activity, wherein the nucleotide sequence contains fewer potential promoter binding sites relative to SEQ ID NO: 16. In other embodiments, the nucleotide sequence that encodes a polypeptide with FVIII activity contains at most 9, at most 8, at most 7, at most 6, or at most 5 potential promoter binding sites. In other embodiments, the nucleotide sequence that encodes a polypeptide with FVIII activity contains at most 4, at most 3, at most 2, or at most 1 potential promoter binding sites. In yet other embodiments, the nucleotide sequence that encodes a polypeptide with FVIII activity does not contain a potential promoter binding site.

In one particular embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence contains fewer potential promoter binding sites relative to SEQ ID NO: 16. In other embodiments, the nucleotide sequence that encodes a polypeptide with FVIII activity contains at most 9, at most 8, at most 7, at most 6, or at most 5 potential promoter binding sites. In other embodiments, the nucleotide sequence that encodes a polypeptide with FVIII activity contains at most 4, at most 3, at most 2, or at most 1 potential promoter binding sites. In yet other embodiments, the nucleotide sequence that encodes a polypeptide with FVIII activity does not contain a potential promoter binding site.

In another embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment); or (iv) nucleotides 1792-2277 and 2320-4374 of SEQ ID NO: 6 (i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the nucleotide sequence contains fewer potential promoter binding sites relative to SEQ ID NO: 16. In other embodiments, the nucleotide sequence that encodes a polypeptide with FVIII activity contains at most 9, at most 8, at most 7, at most 6, or at most 5 potential promoter binding sites. In other embodiments, the nucleotide sequence that encodes a polypeptide with FVIII activity contains at most 4, at most 3, at most 2, or at most 1 potential promoter binding sites. In yet other embodiments, the nucleotide sequence that encodes a polypeptide with FVIII activity does not contain a potential promoter binding site.

In other embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the nucleotide sequence contains fewer potential promoter binding sites relative to SEQ ID NO: 16. In other embodiments, the nucleotide sequence that encodes a polypeptide with FVIII activity contains at most 9, at most 8, at most 7, at most 6, or at most 5 potential promoter binding sites. In other embodiments, the nucleotide sequence that encodes a polypeptide with FVIII activity contains at most 4, at most 3, at most 2, or at most 1 potential promoter binding sites. In yet other embodiments, the nucleotide sequence that encodes a polypeptide with FVIII activity does not contain a potential promoter binding site.

TATA boxes are regulatory sequences often found in the promoter regions of eukaryotes. They serve as the binding site of TATA binding protein (TBP), a general transcription factor. TATA boxes usually comprise the sequence TATAA (SEQ ID NO: 28) or a close variant. TATA boxes within a coding sequence, however, can inhibit the translation of full-length protein. There are ten potential promoter binding sequences in the wild type BDD FVIII sequence (SEQ ID NO: 16): five TATAA sequences (SEQ ID NO: 28) and five TTATA sequences (SEQ ID NO: 29). In some embodiments, at least 1, at least 2, at least 3, or at least 4 of the promoter binding sites are abolished in the FVIII genes of the present disclosure. In some embodiments, at least 5 of the promoter binding sites are abolished in the FVIII genes of the present disclosure. In other embodiments, at least 6, at least 7, or at least 8 of the promoter binding sites are abolished in the FVIII genes of the present disclosure. In one embodiment, at least 9 of the promoter binging sites are abolished in the FVIII genes of the present disclosure. In one particular embodiment, all promoter binding sites are abolished in the FVIII genes of the present disclosure. The location of each potential promoter binding site and the sequence of the corresponding nucleotides in the optimized sequences are shown in Table 2.

### Other Cis Acting Negative Regulatory Elements

In addition to the MAR/ARS sequences, destabilizing elements, and potential promoter sites described above, several additional potentially inhibitory sequences can be identified in the wild type BDD FVIII sequence (SEQ ID NO: 16). Two AU rich sequence elements (AREs) can be identified (ATTTTATT (SEQ ID NOs: 30); and ATTTTTAA (SEQ ID NO: 31), along with a poly-A site (AAAAAAA; SEQ ID NO: 26), a poly-T site (TTTTTT; SEQ ID NO: 25), and a splice site (GGTGAT; SEQ ID NO: 27) in the non-optimized BDD FVIII sequence. One or more of these elements can be removed from the optimized FVIII sequences. The location of each of these sites and the sequence of the corresponding nucleotides in the optimized sequences are shown in Table 2.

In certain embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence does not contain one or more cis-acting negative regulatory elements, for example, a splice site, a poly-T sequence, a poly-A sequence, an ARE sequence, or any combinations thereof.

In another embodiment, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment); or (iv) nucleotides 1792-2277 and 2320-4374 of SEQ ID NO: 6 (i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the nucleotide sequence does not contain one or more cis-acting negative regulatory elements, for example, a splice site, a poly-T sequence, a poly-A sequence, an ARE sequence, or any combinations thereof.

In other embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the nucleotide sequence does not contain one or more cis-acting negative regulatory elements, for example, a splice site, a poly-T sequence, a poly-A sequence, an ARE sequence, or any combinations thereof.

In some embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence does not contain the splice site GGTGAT (SEQ ID NO: 27). In some embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence does not contain a poly-T sequence (SEQ ID NO: 25). In some embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence does not contain a poly-A sequence (SEQ ID NO: 26). In some embodiments, the present disclosure provides an isolated nucleic acid molecule comprising a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence does not contain an ARE element (SEQ ID NO: 30 or SEQ ID NO: 31).

In some embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the nucleotide sequence does not contain the splice site GGTGAT (SEQ ID NO: 27). In some embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the nucleotide sequence does not contain a poly-T sequence (SEQ ID NO: 25). In some embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the nucleotide sequence does not contain a poly-A sequence (SEQ ID NO: 26). In some embodiments, the present disclosure provides an isolated nucleic acid molecule comprising a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the nucleotide sequence does not contain an ARE element (SEQ ID NO: 30 or SEQ ID NO: 31).

In other embodiments, an optimized FVIII sequence of the disclosure does not comprise one or more of antiviral motifs, stem-loop structures, and repeat sequences.

In still other embodiments, the nucleotides surrounding the transcription start site are changed to a kozak consensus sequence (GCCGCCACCATGC (SEQ ID NO: 32), wherein the underlined nucleotides are the start codon). In other embodiments, restriction sites can be added or removed to facilitate the cloning process.

### Heterologous Nucleotide Sequences

In some embodiments, the isolated nucleic acid molecules of the disclosure further comprise a heterologous nucleotide sequence. In some embodiments, the isolated nucleic acid molecules of the disclosure further comprise at least one heterologous nucleotide sequence. The heterologous nucleotide sequence can be linked with the optimized BDD-FVIII nucleotide sequences of the disclosure at the 5′ end, at the 3′ end, or inserted into the middle of the optimized BDD-FVIII nucleotide sequence. Thus, in some embodiments, the heterologous amino acid sequence encoded by the heterologous nucleotide sequence is linked to the N-terminus or the C-terminus of the FVIII amino acid sequence encoded by the nucleotide sequence or inserted between two amino acids in the FVIII amino acid sequence. In some embodiments, the heterologous amino acid sequence can be inserted between two amino acids at one or more insertion site selected from Table 3. In some embodiments, the heterologous amino acid sequence can be inserted within the FVIII polypeptide encoded by the nucleic acid molecule of the disclosure at any site disclosed in International Publication No. WO 2013/123457 A1, WO 2015/106052 A1 or U.S. Publication No. 2015/0158929 A1, which are herein incorporated by reference in their entirety.

In some embodiments, the heterologous amino acid sequence encoded by the heterologous nucleotide sequence is inserted within the B domain or a fragment thereof. In some embodiments, the heterologous amino acid sequence is inserted within the FVIII immediately downstream of an amino acid corresponding to amino acid 745 of mature human FVIII (SEQ ID NO:15). In one particular embodiment, the FVIII comprises a deletion of amino acids 746-1646, corresponding to mature human FVIII (SEQ ID NO:15), and the heterologous amino acid sequence encoded by the heterologous nucleotide sequence is inserted immediately downstream of amino acid 745, corresponding to mature human FVIII (SEQ ID NO:15).

TABLE 3
Heterologous Moiety Insertion Sites

| Insertion | |
|---|---|
| Site | Domain |
| 3 | A1 |
| 18 | A1 |
| 22 | A1 |
| 26 | A1 |
| 40 | A1 |
| 60 | A1 |
| 65 | A1 |
| 81 | A1 |
| 116 | A1 |
| 119 | A1 |
| 130 | A1 |
| 188 | A1 |
| 211 | A1 |
| 216 | A1 |
| 220 | A1 |
| 224 | A1 |
| 230 | A1 |
| 333 | A1 |
| 336 | A1 |
| 339 | A1 |
| 375 | A2 |
| 378 | A2 |
| 399 | A2 |
| 403 | A2 |
| 409 | A2 |
| 416 | A2 |
| 442 | A2 |
| 487 | A2 |
| 490 | A2 |
| 494 | A2 |
| 500 | A2 |
| 518 | A2 |
| 599 | A2 |
| 603 | A2 |
| 713 | A2 |
| 745 | B |
| 1656 | a3 |
| | region |
| 1711 | A3 |
| 1720 | A3 |
| 1725 | A3 |
| 1749 | A3 |
| 1796 | A3 |
| 1802 | A3 |
| 1827 | A3 |
| 1861 | A3 |
| 1896 | A3 |
| 1900 | A3 |
| 1904 | A3 |
| 1905 | A3 |
| 1910 | A3 |
| 1937 | A3 |
| 2019 | A3 |
| 2068 | C1 |
| 2111 | C1 |
| 2120 | C1 |
| 2171 | C2 |
| 2188 | C2 |
| 2227 | C2 |
| 2332 | CT |

Note:
Insertion sites indicate the amino acid position corresponding to an amino acid position of mature human FVIII (SEQ ID NO: 15).

In other embodiments, the isolated nucleic acid molecules of the disclosure further comprise two, three, four, five, six, seven, or eight heterologous nucleotide sequences. In some embodiments, all the heterologous nucleotide sequences are identical. In some embodiments, at least one heterologous nucleotide sequence is different from the other heterologous nucleotide sequences. In some embodiments, the disclosure can comprise two, three, four, five, six, or more than seven heterologous nucleotide sequences in tandem.

In some embodiments, the heterologous nucleotide sequence encodes an amino acid sequence. In some embodiments, the amino acid sequence encoded by the heterologous nucleotide sequence is a heterologous moiety that can increase the half-life (a “half-life extender”) of a FVIII molecule.

In some embodiments, the heterologous moiety is a peptide or a polypeptide with either unstructured or structured characteristics that are associated with the prolongation of in vivo half-life when incorporated in a protein of the disclosure. Non-limiting examples include albumin, albumin fragments, Fc fragments of immunoglobulins, the C-terminal peptide (CTP) of the β subunit of human chorionic gonadotropin, a HAP sequence, an XTEN sequence, a transferrin or a fragment thereof, a PAS polypeptide, polyglycine linkers, polyserine linkers, albumin-binding moieties, or any fragments, derivatives, variants, or combinations of these polypeptides. In one particular embodiment, the heterologous amino acid sequence is an immunoglobulin constant region or a portion thereof, transferrin, albumin, or a PAS sequence. In some aspects, a heterologous moiety includes von Willebrand factor or a fragment thereof. In other related aspects a heterologous moiety can include an attachment site (e.g., a cysteine amino acid) for a non-polypeptide moiety such as polyethylene glycol (PEG), hydroxyethyl starch (HES), polysialic acid, or any derivatives, variants, or combinations of these elements. In some aspects, a heterologous moiety comprises a cysteine amino acid that functions as an attachment site for a non-polypeptide moiety such as polyethylene glycol (PEG), hydroxyethyl starch (HES), polysialic acid, or any derivatives, variants, or combinations of these elements.

In one specific embodiment, a first heterologous nucleotide sequence encodes a first heterologous moiety that is a half-life extending molecule which is known in the art, and a second heterologous nucleotide sequence encodes a second heterologous moiety that can also be a half-life extending molecule which is known in the art. In certain embodiments, the first heterologous moiety (e.g., a first Fc moiety) and the second heterologous moiety (e.g., a second Fc moiety) are associated with each other to form a dimer. In one embodiment, the second heterologous moiety is a second Fc moiety, wherein the second Fc moiety is linked to or associated with the first heterologous moiety, e.g., the first Fc moiety. For example, the second heterologous moiety (e.g., the second Fc moiety) can be linked to the first heterologous moiety (e.g., the first Fc moiety) by a linker or associated with the first heterologous moiety by a covalent or non-covalent bond.

In some embodiments, the heterologous moiety is a polypeptide comprising, consisting essentially of, or consisting of at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2500, at least about 3000, or at least about 4000 amino acids. In other embodiments, the heterologous moiety is a polypeptide comprising, consisting essentially of, or consisting of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids.

In certain embodiments, a heterologous moiety improves one or more pharmacokinetic properties of the FVIII protein without significantly affecting its biological activity or function.

In certain embodiments, a heterologous moiety increases the in vivo and/or in vitro half-life of the FVIII protein of the disclosure. In other embodiments, a heterologous moiety facilitates visualization or localization of the FVIII protein of the disclosure or a fragment thereof (e.g., a fragment comprising a heterologous moiety after proteolytic cleavage of the FVIII protein). Visualization and/or location of the FVIII protein of the disclosure or a fragment thereof can be in vivo, in vitro, ex vivo, or combinations thereof.

In other embodiments, a heterologous moiety increases stability of the FVIII protein of the disclosure or a fragment thereof (e.g., a fragment comprising a heterologous moiety after proteolytic cleavage of the FVIII protein). As used herein, the term “stability” refers to an art-recognized measure of the maintenance of one or more physical properties of the FVIII protein in response to an environmental condition (e.g., an elevated or lowered temperature). In certain aspects, the physical property can be the maintenance of the covalent structure of the FVIII protein (e.g., the absence of proteolytic cleavage, unwanted oxidation or deamidation). In other aspects, the physical property can also be the presence of the FVIII protein in a properly folded state (e.g., the absence of soluble or insoluble aggregates or precipitates). In one aspect, the stability of the FVIII protein is measured by assaying a biophysical property of the FVIII protein, for example thermal stability, pH unfolding profile, stable removal of glycosylation, solubility, biochemical function (e.g., ability to bind to a protein, receptor or ligand), etc., and/or combinations thereof. In another aspect, biochemical function is demonstrated by the binding affinity of the interaction. In one aspect, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

In certain aspects, a FVIII protein encoded by the nucleic acid molecule of the disclosure comprises at least one half-life extender, i.e., a heterologous moiety which increases the in vivo half-life of the FVIII protein with respect to the in vivo half-life of the corresponding FVIII protein lacking such heterologous moiety. In vivo half-life of a FVIII protein can be determined by any methods known to those of skill in the art, e.g., activity assays (chromogenic assay or one stage clotting aPTT assay), ELISA, ROTEM™, etc.

In some embodiments, the presence of one or more half-life extenders results in the half-life of the FVIII protein to be increased compared to the half-life of the corresponding protein lacking such one or more half-life extenders. The half-life of the FVIII protein comprising a half-life extender is at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than the in vivo half-life of the corresponding FVIII protein lacking such half-life extender.

In one embodiment, the half-life of the FVIII protein comprising a half-life extender is about 1.5-fold to about 20-fold, about 1.5 fold to about 15 fold, or about 1.5 fold to about 10 fold longer than the in vivo half-life of the corresponding protein lacking such half-life extender. In another embodiment, the half-life of FVIII protein comprising a half-life extender is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to the in vivo half-life of the corresponding protein lacking such half-life extender.

In other embodiments, the half-life of the FVIII protein comprising a half-life extender is at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 48 hours, at least about 60 hours, at least about 72 hours, at least about 84 hours, at least about 96 hours, or at least about 108 hours.

In still other embodiments, the half-life of the FVIII protein comprising a half-life extender is about 15 hours to about two weeks, about 16 hours to about one week, about 17 hours to about one week, about 18 hours to about one week, about 19 hours to about one week, about 20 hours to about one week, about 21 hours to about one week, about 22 hours to about one week, about 23 hours to about one week, about 24 hours to about one week, about 36 hours to about one week, about 48 hours to about one week, about 60 hours to about one week, about 24 hours to about six days, about 24 hours to about five days, about 24 hours to about four days, about 24 hours to about three days, or about 24 hours to about two days.

In some embodiments, the average half-life per subject of the FVIII protein comprising a half-life extender is about 15 hours, about 16 hours, about 17 hours, about 18 hours, about 19 hours, about 20 hours, about 21 hours, about 22 hours, about 23 hours, about 24 hours (1 day), about 25 hours, about 26 hours, about 27 hours, about 28 hours, about 29 hours, about 30 hours, about 31 hours, about 32 hours, about 33 hours, about 34 hours, about 35 hours, about 36 hours, about 40 hours, about 44 hours, about 48 hours (2 days), about 54 hours, about 60 hours, about 72 hours (3 days), about 84 hours, about 96 hours (4 days), about 108 hours, about 120 hours (5 days), about six days, about seven days (one week), about eight days, about nine days, about 10 days, about 11 days, about 12 days, about 13 days, or about 14 days.

One or more half-life extenders can be fused to C-terminus or N-terminus of FVIII or inserted within FVIII.

1. an Immunoglobulin Constant Region or a Portion Thereof

In another aspect, a heterologous moiety comprises one or more immunoglobulin constant regions or portions thereof (e.g., an Fc region). In one embodiment, an isolated nucleic acid molecule of the disclosure further comprises a heterologous nucleic acid sequence that encodes an immunoglobulin constant region or a portion thereof. In some embodiments, the immunoglobulin constant region or portion thereof is an Fc region.

An immunoglobulin constant region is comprised of domains denoted CH (constant heavy) domains (CH1, CH2, etc.). Depending on the isotype, (i.e. IgG, IgM, IgA IgD, or IgE), the constant region can be comprised of three or four CH domains. Some isotypes (e.g. IgG) constant regions also contain a hinge region. See Janeway et al. 2001, Immunobiology, Garland Publishing, N.Y., N.Y.

An immunoglobulin constant region or a portion thereof for producing the FVIII protein of the present disclosure can be obtained from a number of different sources. In one embodiment, an immunoglobulin constant region or a portion thereof is derived from a human immunoglobulin. It is understood, however, that the immunoglobulin constant region or a portion thereof can be derived from an immunoglobulin of another mammalian species, including for example, a rodent (e.g., a mouse, rat, rabbit, guinea pig) or non-human primate (e.g., chimpanzee, macaque) species. Moreover, the immunoglobulin constant region or a portion thereof can be derived from any immunoglobulin class, including IgM, IgG, IgD, IgA and IgE, and any immunoglobulin isotype, including IgG1, IgG2, IgG3 and IgG4. In one embodiment, the human isotype IgG1 is used.

A variety of the immunoglobulin constant region gene sequences (e.g., human constant region gene sequences) are available in the form of publicly accessible deposits. Constant region domains sequence can be selected having a particular effector function (or lacking a particular effector function) or with a particular modification to reduce immunogenicity. Many sequences of antibodies and antibody-encoding genes have been published and suitable Ig constant region sequences (e.g., hinge, CH2, and/or CH3 sequences, or portions thereof) can be derived from these sequences using art recognized techniques. The genetic material obtained using any of the foregoing methods can then be altered or synthesized to obtain polypeptides of the present disclosure. It will further be appreciated that the scope of this disclosure encompasses alleles, variants and mutations of constant region DNA sequences.

The sequences of the immunoglobulin constant region or a portion thereof can be cloned, e.g., using the polymerase chain reaction and primers which are selected to amplify the domain of interest. To clone a sequence of the immunoglobulin constant region or a portion thereof from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, C A (1990); Ho et al. 1989. Gene 77:51; Horton et al. 1993. Methods Enzymol. 217:270). PCR can be initiated by consensus constant region primers or by more specific primers based on the published heavy and light chain DNA and amino acid sequences. PCR also can be used to isolate DNA clones encoding the antibody light and heavy chains. In this case the libraries can be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250). The cloning of antibody sequences is further described in Newman et al., U.S. Pat. No. 5,658,570, filed Jan. 25, 1995, which is incorporated by reference herein.

An immunoglobulin constant region used herein can include all domains and the hinge region or portions thereof. In one embodiment, the immunoglobulin constant region or a portion thereof comprises CH2 domain, CH3 domain, and a hinge region, i.e., an Fc region or an FcRn binding partner.

As used herein, the term “Fc region” is defined as the portion of a polypeptide which corresponds to the Fc region of native Ig, i.e., as formed by the dimeric association of the respective Fc domains of its two heavy chains. A native Fc region forms a homodimer with another Fc region. In contrast, the term “genetically-fused Fc region” or “single-chain Fc region” (scFc region), as used herein, refers to a synthetic dimeric Fc region comprised of Fc domains genetically linked within a single polypeptide chain (i.e., encoded in a single contiguous genetic sequence). See International Publication No. WO 2012/006635, incorporated herein by reference in its entirety.

In one embodiment, the “Fc region” refers to the portion of a single Ig heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge domain, a CH2 domain, and a CH3 domain.

An immunoglobulin constant region or a portion thereof can be an FcRn binding partner. FcRn is active in adult epithelial tissues and expressed in the lumen of the intestines, pulmonary airways, nasal surfaces, vaginal surfaces, colon and rectal surfaces (U.S. Pat. No. 6,485,726). An FcRn binding partner is a portion of an immunoglobulin that binds to FcRn.

The FcRn receptor has been isolated from several mammalian species including humans. The sequences of the human FcRn, monkey FcRn, rat FcRn, and mouse FcRn are known (Story et al. 1994, J. Exp. Med. 180:2377). The FcRn receptor binds IgG (but not other immunoglobulin classes such as IgA, IgM, IgD, and IgE) at relatively low pH, actively transports the IgG transcellularly in a luminal to serosal direction, and then releases the IgG at relatively higher pH found in the interstitial fluids. It is expressed in adult epithelial tissue (U.S. Pat. Nos. 6,485,726, 6,030,613, 6,086,875; WO 03/077834; US2003-0235536A1) including lung and intestinal epithelium (Israel et al. 1997, Immunology 92:69) renal proximal tubular epithelium (Kobayashi et al. 2002, Am. J. Physiol. Renal Physiol. 282:F358) as well as nasal epithelium, vaginal surfaces, and biliary tree surfaces.

FcRn binding partners useful in the present disclosure encompass molecules that can be specifically bound by the FcRn receptor including whole IgG, the Fc fragment of IgG, and other fragments that include the complete binding region of the FcRn receptor. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The FcRn binding partners include whole IgG, the Fc fragment of IgG, and other fragments of IgG that include the complete binding region of FcRn. The major contact sites include amino acid residues 248, 250-257, 272, 285, 288, 290-291, 308-311, and 314 of the CH2 domain and amino acid residues 385-387, 428, and 433-436 of the CH3 domain. References made to amino acid numbering of immunoglobulins or immunoglobulin fragments, or regions, are all based on Kabat et al. 1991, Sequences of Proteins of Immunological Interest, U.S. Department of Public Health, Bethesda, Md.

Fc regions or FcRn binding partners bound to FcRn can be effectively shuttled across epithelial barriers by FcRn, thus providing a non-invasive means to systemically administer a desired therapeutic molecule. Additionally, fusion proteins comprising an Fc region or an FcRn binding partner are endocytosed by cells expressing the FcRn. But instead of being marked for degradation, these fusion proteins are recycled out into circulation again, thus increasing the in vivo half-life of these proteins. In certain embodiments, the portions of immunoglobulin constant regions are an Fc region or an FcRn binding partner that typically associates, via disulfide bonds and other non-specific interactions, with another Fc region or another FcRn binding partner to form dimers and higher order multimers.

Two FcRn receptors can bind a single Fc molecule. Crystallographic data suggest that each FcRn molecule binds a single polypeptide of the Fc homodimer. In one embodiment, linking the FcRn binding partner, e.g., an Fc fragment of an IgG, to a biologically active molecule provides a means of delivering the biologically active molecule orally, buccally, sublingually, rectally, vaginally, as an aerosol administered nasally or via a pulmonary route, or via an ocular route. In another embodiment, the FVIII protein can be administered invasively, e.g., subcutaneously, intravenously.

An FcRn binding partner region is a molecule or portion thereof that can be specifically bound by the FcRn receptor with consequent active transport by the FcRn receptor of the Fc region. Specifically bound refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant KA is higher than 10⁶ M⁻¹, or higher than 10⁸ M⁻¹. If necessary, non-specific binding can be reduced without substantially affecting specific binding by varying the binding conditions. The appropriate binding conditions such as concentration of the molecules, ionic strength of the solution, temperature, time allowed for binding, concentration of a blocking agent (e.g., serum albumin, milk casein), etc., can be optimized by a skilled artisan using routine techniques.

In certain embodiments, a FVIII protein encoded by the nucleic acid molecule of the disclosure comprises one or more truncated Fc regions that are nonetheless sufficient to confer Fc receptor (FcR) binding properties to the Fc region. For example, the portion of an Fc region that binds to FcRn (i.e., the FcRn binding portion) comprises from about amino acids 282-438 of IgG1, EU numbering (with the primary contact sites being amino acids 248, 250-257, 272, 285, 288, 290-291, 308-311, and 314 of the CH2 domain and amino acid residues 385-387, 428, and 433-436 of the CH3 domain. Thus, an Fc region of the disclosure can comprise or consist of an FcRn binding portion. FcRn binding portions can be derived from heavy chains of any isotype, including IgGI, IgG2, IgG3 and IgG4. In one embodiment, an FcRn binding portion from an antibody of the human isotype IgG1 is used. In another embodiment, an FcRn binding portion from an antibody of the human isotype IgG4 is used.

The Fc region can be obtained from a number of different sources. In one embodiment, an Fc region of the polypeptide is derived from a human immunoglobulin. It is understood, however, that an Fc moiety can be derived from an immunoglobulin of another mammalian species, including for example, a rodent (e.g., a mouse, rat, rabbit, guinea pig) or non-human primate (e.g., chimpanzee, macaque) species. Moreover, the polypeptide of the Fc domains or portions thereof can be derived from any immunoglobulin class, including IgM, IgG, IgD, IgA and IgE, and any immunoglobulin isotype, including IgG1, IgG2, IgG3 and IgG4. In another embodiment, the human isotype IgG1 is used.

In certain embodiments, the Fc variant confers a change in at least one effector function imparted by an Fc moiety comprising said wild-type Fc domain (e.g., an improvement or reduction in the ability of the Fc region to bind to Fc receptors (e.g. FcγRI, FcγRII, or FcγRIII) or complement proteins (e.g. C1q), or to trigger antibody-dependent cytotoxicity (ADCC), phagocytosis, or complement-dependent cytotoxicity (CDCC)). In other embodiments, the Fc variant provides an engineered cysteine residue.

The Fc region of the disclosure can employ art-recognized Fc variants which are known to impart a change (e.g., an enhancement or reduction) in effector function and/or FcR or FcRn binding. Specifically, an Fc region of the disclosure can include, for example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, US2007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091, each of which is incorporated by reference herein. In one embodiment, the specific change (e.g., the specific substitution of one or more amino acids disclosed in the art) can be made at one or more of the disclosed amino acid positions. In another embodiment, a different change at one or more of the disclosed amino acid positions (e.g., the different substitution of one or more amino acid position disclosed in the art) can be made.

The Fc region or FcRn binding partner of IgG can be modified according to well recognized procedures such as site directed mutagenesis and the like to yield modified IgG or Fc fragments or portions thereof that will be bound by FcRn. Such modifications include modifications remote from the FcRn contact sites as well as modifications within the contact sites that preserve or even enhance binding to the FcRn. For example, the following single amino acid residues in human IgG1 Fc (Fc γ1) can be substituted without significant loss of Fc binding affinity for FcRn: P238A, S239A, K246A, K248A, D249A, M252A, T256A, E258A, T260A, D265A, S267A, H268A, E269A, D270A, E272A, L274A, N276A, Y278A, D280A, V282A, E283A, H285A, N286A, T289A, K290A, R292A, E293A, E294A, Q295A, Y296F, N297A, S298A, Y300F, R301A, V303A, V305A, T307A, L309A, Q311A, D312A, N315A, K317A, E318A, K320A, K322A, S324A, K326A, A327Q, P329A, A330Q, P331A, E333A, K334A, T335A, S337A, K338A, K340A, Q342A, R344A, E345A, Q347A, R355A, E356A, M358A, T359A, K360A, N361A, Q362A, Y373A, S375A, D376A, A378Q, E380A, E382A, S383A, N384A, Q386A, E388A, N389A, N390A, Y391F, K392A, L398A, S400A, D401A, D413A, K414A, R416A, Q418A, Q419A, N421A, V422A, S424A, E430A, N434A, T437A, Q438A, K439A, S440A, S444A, and K447A, where for example P238A represents wild type proline substituted by alanine at position number 238. As an example, a specific embodiment incorporates the N297A mutation, removing a highly conserved N-glycosylation site. In addition to alanine other amino acids can be substituted for the wild type amino acids at the positions specified above. Mutations can be introduced singly into Fc giving rise to more than one hundred Fc regions distinct from the native Fc. Additionally, combinations of two, three, or more of these individual mutations can be introduced together, giving rise to hundreds more Fc regions.

Certain of the above mutations can confer new functionality upon the Fc region or FcRn binding partner. For example, one embodiment incorporates N297A, removing a highly conserved N-glycosylation site. The effect of this mutation is to reduce immunogenicity, thereby enhancing circulating half-life of the Fc region, and to render the Fc region incapable of binding to FcγRI, FcγRIIA, FcγRIIB, and FcγRIIIA, without compromising affinity for FcRn (Routledge et al. 1995, Transplantation 60:847; Friend et al. 1999, Transplantation 68:1632; Shields et al. 1995, J. Biol. Chem. 276:6591). As a further example of new functionality arising from mutations described above affinity for FcRn can be increased beyond that of wild type in some instances. This increased affinity can reflect an increased “on” rate, a decreased “off” rate or both an increased “on” rate and a decreased “off” rate. Examples of mutations believed to impart an increased affinity for FcRn include, but not limited to, T256A, T307A, E380A, and N434A (Shields et al. 2001, J. Biol. Chem. 276:6591).

Additionally, at least three human Fc gamma receptors appear to recognize a binding site on IgG within the lower hinge region, generally amino acids 234-237. Therefore, another example of new functionality and potential decreased immunogenicity can arise from mutations of this region, as for example by replacing amino acids 233-236 of human IgG1 “ELLG” (SEQ ID NO: 45) to the corresponding sequence from IgG2 “PVA” (with one amino acid deletion). It has been shown that FcγRI, FcγRII, and FcγRIII, which mediate various effector functions will not bind to IgG1 when such mutations have been introduced. Ward and Ghetie 1995, Therapeutic Immunology 2:77 and Armour et al. 1999, Eur. J. Immunol. 29:2613.

In another embodiment, the immunoglobulin constant region or a portion thereof comprises an amino acid sequence in the hinge region or a portion thereof that forms one or more disulfide bonds with a second immunoglobulin constant region or a portion thereof. The second immunoglobulin constant region or a portion thereof can be linked to a second polypeptide, bringing the FVIII protein and the second polypeptide together. In some embodiments, the second polypeptide is an enhancer moiety. As used herein, the term “enhancer moiety” refers to a molecule, fragment thereof or a component of a polypeptide which is capable of enhancing the procoagulant activity of FVIII. The enhancer moiety can be a cofactor, such as soluble tissue factor (sTF), or a procoagulant peptide. Thus, upon activation of FVIII, the enhancer moiety is available to enhance FVIII activity.

In certain embodiments, a FVIII protein encoded by a nucleic acid molecule of the disclosure comprises an amino acid substitution to an immunoglobulin constant region or a portion thereof (e.g., Fc variants), which alters the antigen-independent effector functions of the Ig constant region, in particular the circulating half-life of the protein.

2. scFc Regions

In another aspect, a heterologous moiety comprises a scFc (single chain Fc) region. In one embodiment, an isolated nucleic acid molecule of the disclosure further comprises a heterologous nucleic acid sequence that encodes a scFc region. The scFc region comprises at least two immunoglobulin constant regions or portions thereof (e.g., Fc moieties or domains (e.g., 2, 3, 4, 5, 6, or more Fc moieties or domains)) within the same linear polypeptide chain that are capable of folding (e.g., intramolecularly or intermolecularly folding) to form one functional scFc region which is linked by an Fc peptide linker. For example, in one embodiment, a polypeptide of the disclosure is capable of binding, via its scFc region, to at least one Fc receptor (e.g., an FcRn, an FcγR receptor (e.g., FcγRIII), or a complement protein (e.g., C1q)) in order to improve half-life or trigger an immune effector function (e.g., antibody-dependent cytotoxicity (ADCC), phagocytosis, or complement-dependent cytotoxicity (CDCC) and/or to improve manufacturability).

3. CTP

In another aspect, a heterologous moiety comprises one C-terminal peptide (CTP) of the β subunit of human chorionic gonadotropin or fragment, variant, or derivative thereof. One or more CTP peptides inserted into a recombinant protein is known to increase the in vivo half-life of that protein. See, e.g., U.S. Pat. No. 5,712,122, incorporated by reference herein in its entirety.

Exemplary CTP peptides include DPRFQDSSSSKAPPPSLPSPSRLPGPSDTPIL (SEQ ID NO: 33) or SSSSKAPPPSLPSPSRLPGPSDTPILPQ (SEQ ID NO: 34). See, e.g., U.S. Patent Application Publication No. US 2009/0087411 A1, incorporated by reference.

4. XTEN Sequence

In some embodiments, a heterologous moiety comprises one or more XTEN sequences, fragments, variants, or derivatives thereof. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a heterologous moiety, XTENs can serve as a half-life extension moiety. In addition, XTEN can provide desirable properties including but are not limited to enhanced pharmacokinetic parameters and solubility characteristics.

The incorporation of a heterologous moiety comprising an XTEN sequence into a protein of the disclosure can confer to the protein one or more of the following advantageous properties: conformational flexibility, enhanced aqueous solubility, high degree of protease resistance, low immunogenicity, low binding to mammalian receptors, or increased hydrodynamic (or Stokes) radii.

In certain aspects, an XTEN sequence can increase pharmacokinetic properties such as longer in vivo half-life or increased area under the curve (AUC), so that a protein of the disclosure stays in vivo and has procoagulant activity for an increased period of time compared to a protein with the same but without the XTEN heterologous moiety.

In some embodiments, the XTEN sequence useful for the disclosure is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues. In one particular embodiment, the XTEN comprises an amino acid sequence of longer than 42 amino acids and shorter than 144 amino acids in length.

The XTEN sequence of the disclosure can comprise one or more sequence motif of 5 to 14 (e.g., 9 to 14) amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids (e.g., 5 amino acids) selected from the group consisting of glycine (G), alanine (A), serine (S), threonine (T), glutamate (E) and proline (P). See US 2010-0239554 A1.

In some embodiments, the XTEN comprises non-overlapping sequence motifs in which about 80%, or at least about 85%, or at least about 90%, or about 91%, or about 92%, or about 93%, or about 94%, or about 95%, or about 96%, or about 97%, or about 98%, or about 99% or about 100% of the sequence consists of multiple units of non-overlapping sequences selected from a single motif family selected from Table 4, resulting in a family sequence. As used herein, “family” means that the XTEN has motifs selected only from a single motif category from Table 4; i.e., AD, AE, AF, AG, AM, AQ, BC, or BD XTEN, and that any other amino acids in the XTEN not from a family motif are selected to achieve a needed property, such as to permit incorporation of a restriction site by the encoding nucleotides, incorporation of a cleavage sequence, or to achieve a better linkage to FVIII. In some embodiments of XTEN families, an XTEN sequence comprises multiple units of non-overlapping sequence motifs of the AD motif family, or of the AE motif family, or of the AF motif family, or of the AG motif family, or of the AM motif family, or of the AQ motif family, or of the BC family, or of the BD family, with the resulting XTEN exhibiting the range of homology described above. In other embodiments, the XTEN comprises multiple units of motif sequences from two or more of the motif families of Table 2A. These sequences can be selected to achieve desired physical/chemical characteristics, including such properties as net charge, hydrophilicity, lack of secondary structure, or lack of repetitiveness that are conferred by the amino acid composition of the motifs, described more fully below. In the embodiments hereinabove described in this paragraph, the motifs incorporated into the XTEN can be selected and assembled using the methods described herein to achieve an XTEN of about 36 to about 3000 amino acid residues.

TABLE 4
XTEN Sequence Motifs of 12 Amino Acids and
Motif Families

| Motif | | |
|---|---|---|
| Family* | MOTIF SEQUENCE | SEQ ID NO: |
| AD | GESPGGSSGSES | 73 |
| AD | GSEGSSGPGESS | 74 |
| AD | GSSESGSSEGGP | 75 |
| AD | GSGGEPSESGSS | 76 |
| AE, AM | GSPAGSPTSTEE | 77 |
| AE, AM, AQ | GSEPATSGSETP | 78 |
| AE, AM, AQ | GTSESATPESGP | 79 |
| AE, AM, AQ | GTSTEPSEGSAP | 80 |
| AF, AM | GSTSESPSGTAP | 81 |
| AF, AM | GTSTPESGSASP | 82 |
| AF, AM | GTSPSGESSTAP | 83 |
| AF, AM | GSTSSTAESPGP | 84 |
| AG, AM | GTPGSGTASSSP | 85 |
| AG, AM | GSSTPSGATGSP | 86 |
| AG, AM | GSSPSASTGTGP | 87 |
| AG, AM | GASPGTSSTGSP | 88 |
| AQ | GEPAGSPTSTSE | 89 |
| AQ | GTGEPSSTPASE | 90 |
| AQ | GSGPSTESAPTE | 91 |
| AQ | GSETPSGPSETA | 92 |
| AQ | GPSETSTSEPGA | 93 |
| AQ | GSPSEPTEGTSA | 94 |
| BC | GSGASEPTSTEP | 95 |
| BC | GSEPATSGTEPS | 96 |
| BC | GTSEPSTSEPGA | 97 |
| BC | GTSTEPSEPGSA | 98 |
| BD | GSTAGSETSTEA | 99 |
| BD | GSETATSGSETA | 100 |
| BD | GTSESATSESGA | 101 |
| BD | GTSTEASEGSAS | 102 |

*Denotes individual motif sequences that, when used together in various permutations, results in a ″family sequence″

Examples of XTEN sequences that can be used as heterologous moieties in chimeric proteins of the disclosure are disclosed, e.g., in U.S. Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, or WO 2011028344 A2, each of which is incorporated by reference herein in its entirety.

XTEN can have varying lengths for insertion into or linkage to FVIII. In one embodiment, the length of the XTEN sequence(s) is chosen based on the property or function to be achieved in the fusion protein. Depending on the intended property or function, XTEN can be short or intermediate length sequence or longer sequence that can serve as carriers. In certain embodiments, the XTEN includes short segments of about 6 to about 99 amino acid residues, intermediate lengths of about 100 to about 399 amino acid residues, and longer lengths of about 400 to about 1000 and up to about 3000 amino acid residues. Thus, the XTEN inserted into or linked to FVIII can have lengths of about 6, about 12, about 36, about 40, about 42, about 72, about 96, about 144, about 288, about 400, about 500, about 576, about 600, about 700, about 800, about 864, about 900, about 1000, about 1500, about 2000, about 2500, or up to about 3000 amino acid residues in length. In other embodiments, the XTEN sequences is about 6 to about 50, about 50 to about 100, about 100 to 150, about 150 to 250, about 250 to 400, about 400 to about 500, about 500 to about 900, about 900 to 1500, about 1500 to 2000, or about 2000 to about 3000 amino acid residues in length. The precise length of an XTEN inserted into or linked to FVIII can vary without adversely affecting the activity of the FVIII. In one embodiment, one or more of the XTENs used herein have 42 amino acids, 72 amino acids, 144 amino acids, 288 amino acids, 576 amino acids, or 864 amino acids in length and can be selected from one or more of the XTEN family sequences; i.e., AD, AE, AF, AG, AM, AQ, BC or BD.

In some embodiments, the XTEN sequence used in the disclosure is at least 60%, 70%, 80%, 85%, 90%, 91%, 92%, 93%, 94%, 95%, 96%, 97%, 98%, 99%, or 100% identical to a sequence selected from the group consisting of AE42, AG42, AE48, AM48, AE72, AG72, AE108, AG108, AE144, AF144, AG144, AE180, AG180, AE216, AG216, AE252, AG252, AE288, AG288, AE324, AG324, AE360, AG360, AE396, AG396, AE432, AG432, AE468, AG468, AE504, AG504, AF504, AE540, AG540, AF540, AD576, AE576, AF576, AG576, AE612, AG612, AE624, AE648, AG648, AG684, AE720, AG720, AE756, AG756, AE792, AG792, AE828, AG828, AD836, AE864, AF864, AG864, AM875, AE912, AM923, AM1318, BC864, BD864, AE948, AE1044, AE1140, AE1236, AE1332, AE1428, AE1524, AE1620, AE1716, AE1812, AE1908, AE2004A, AG948, AG1044, AG1140, AG1236, AG1332, AG1428, AG1524, AG1620, AG1716, AG1812, AG1908, AG2004, and any combination thereof. See US 2010-0239554 A1. In one particular embodiment, the XTEN comprises AE42, AE72, AE144, AE288, AE576, AE864, AG 42, AG72, AG144, AG288, AG576, AG864, or any combination thereof.

Exemplary XTEN sequences that can be used as heterologous moieties in chimeric protein of the disclosure include XTEN AE42-4 (SEQ ID NO: 46, encoded by SEQ ID NO: 47; FIGS. 11C and 11D, respectively), XTEN 144-2A (SEQ ID NO: 48, encoded by SEQ ID NO: 49; FIGS. 11E and 11F, respectively), XTEN A144-31 (SEQ ID NO: 50, encoded by SEQ ID NO: 51; FIGS. 11G and 11H, respectively), XTEN AE144-4A (SEQ ID NO: 52, encoded by SEQ ID NO: 53; FIGS. 11K and 11J, respectively), XTEN AE144-5A (SEQ ID NO: 54, encoded by SEQ ID NO: 55; FIGS. 11K and 11L, respectively), XTEN AE144-6B (SEQ ID NO: 56, encoded by SEQ ID NO: 57; FIGS. 11M and 11N, respectively), XTEN AG144-1 (SEQ ID NO: 58, encoded by SEQ ID NO: 59; FIGS. 11O and 11P, respectively), XTEN AG144-A (SEQ ID NO: 60, encoded by SEQ ID NO: 61; FIGS. 11Q and 11R, respectively), XTEN AG144-B (SEQ ID NO: 62, encoded by SEQ ID NO: 63; FIGS. 11S and 11T, respectively), XTEN AG144-C(SEQ ID NO: 64, encoded by SEQ ID NO: 65; FIGS. 11U and 11V, respectively), and XTEN AG144-F (SEQ ID NO: 66, encoded by SEQ ID NO: 67; FIGS. 11W and 11X, respectively). In one particular embodiment, the XTEN is encoded by SEQ ID NO:18.

In some embodiments, less than 100% of amino acids of an XTEN are selected from glycine (G), alanine (A), serine (S), threonine (T), glutamate (E) and proline (P), or less than 100% of the sequence consists of the sequence motifs from Table 2A or an XTEN sequence provided herein. In such embodiments, the remaining amino acid residues of the XTEN are selected from any of the other 14 natural L-amino acids, but can be preferentially selected from hydrophilic amino acids such that the XTEN sequence contains at least about 90%, 91%, 92%, 93%, 94%, 95%, 96%, 97%, 98%, or at least about 99% hydrophilic amino acids. The content of hydrophobic amino acids in the XTEN utilized in the conjugation constructs can be less than 5%, or less than 2%, or less than 1% hydrophobic amino acid content. Hydrophobic residues that are less favored in construction of XTEN include tryptophan, phenylalanine, tyrosine, leucine, isoleucine, valine, and methionine. Additionally, XTEN sequences can contain less than 5% or less than 4% or less than 3% or less than 2% or less than 1% or none of the following amino acids: methionine (for example, to avoid oxidation), or asparagine and glutamine (to avoid desamidation).

The one or more XTEN sequences can be inserted at the C-terminus or at the N-terminus of the amino acid sequence encoded by the nucleotide sequence or inserted between two amino acids in the amino acid sequence encoded by the nucleotide sequence. For example, the XTEN can be inserted between two amino acids at one or more insertion site selected from Table 3. Examples of sites within FVIII that are permissible for XTEN insertion can be found in, e.g., International Publication No. WO 2013/123457 A1 or U.S. Publication No. 2015/0158929 A1, which are herein incorporated by reference in their entirety.

5. Albumin or Fragment, Derivative, or Variant Thereof

In some embodiments, a heterologous moiety comprises albumin or a functional fragment thereof. Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ. Nos. 2008/0194481A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2, which are incorporated herein by reference in their entireties.

In one embodiment, the FVIII protein encoded by a nucleic acid molecule of the disclosure comprises albumin, a fragment, or a variant thereof which is further linked to a second heterologous moiety selected from the group consisting of an immunoglobulin constant region or portion thereof (e.g., an Fc region), a PAS sequence, HES, and PEG.

6. Albumin-Binding Moiety

In certain embodiments, the heterologous moiety is an albumin-binding moiety, which comprises an albumin-binding peptide, a bacterial albumin-binding domain, an albumin-binding antibody fragment, or any combinations thereof.

For example, the albumin-binding protein can be a bacterial albumin-binding protein, an antibody or an antibody fragment including domain antibodies (see U.S. Pat. No. 6,696,245). An albumin-binding protein, for example, can be a bacterial albumin-binding domain, such as the one of streptococcal protein G (Konig, T. and Skerra, A. (1998) J. Immunol. Methods 218, 73-83). Other examples of albumin-binding peptides that can be used as conjugation partner are, for instance, those having a Cys-Xaa₁-Xaa₂-Xaa₃-Xaa₄-Cys consensus sequence, wherein Xaa₁ is Asp, Asn, Ser, Thr, or Trp; Xaa₂ is Asn, Gln, H is, lie, Leu, or Lys; Xaa₃ is Ala, Asp, Phe, Trp, or Tyr; and Xaa₄ is Asp, Gly, Leu, Phe, Ser, or Thr as described in US patent application 2003/0069395 or Dennis et al. (Dennis et al. (2002) J. Biol. Chem. 277, 35035-35043).

Domain 3 from streptococcal protein G, as disclosed by Kraulis et al., FEBS Lett. 378:190-194 (1996) and Linhult et al., Protein Sci. 11:206-213 (2002) is an example of a bacterial albumin-binding domain. Examples of albumin-binding peptides include a series of peptides having the core sequence DICLPRWGCLW (SEQ ID NO: 35). See, e.g., Dennis et al., J. Biol. Chem. 2002, 277: 35035-35043 (2002). Examples of albumin-binding antibody fragments are disclosed in Muller and Kontermann, Curr. Opin. Mol. Ther. 9:319-326 (2007); Roovers et al., Cancer Immunol. Immunother. 56:303-317 (2007), and Holt et al., Prot. Eng. Design Sci., 21:283-288 (2008), which are incorporated herein by reference in their entireties. An example of such albumin-binding moiety is 2-(3-maleimidopropanamido)-6-(4-(4-iodophenyl)butanamido) hexanoate (“Albu” tag) as disclosed by Trussel et al., Bioconjugate Chem. 20:2286-2292 (2009).

Fatty acids, in particular long chain fatty acids (LCFA) and long chain fatty acid-like albumin-binding compounds can be used to extend the in vivo half-life of FVIII proteins of the disclosure. An example of a LCFA-like albumin-binding compound is 16-(1-(3-(9-(((2,5-dioxopyrrolidin-1-yloxy) carbonyloxy)-methyi)-7-sulfo-9H-fluoren-2-ylamino)-3-oxopropyl)-2,5-dioxopyrrolidin-3-ylthio) hexadecanoic acid (see, e.g., WO 2010/140148).

7. PAS Sequence

In other embodiments, the heterologous moiety is a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the chimeric protein. Yet, the skilled person is aware that an amino acid polymer also can form random coil conformation when residues other than alanine, serine, and proline are added as a minor constituent in the PAS sequence. The term “minor constituent” as used herein means that amino acids other than alanine, serine, and proline can be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids. The amino acids different from alanine, serine and proline can be selected from the group consisting of Arg, Asn, Asp, Cys, Gln, Glu, Gly, His, lie, Leu, Lys, Met, Phe, Thr, Trp, Tyr, and Val.

Under physiological conditions, the PAS sequence stretch forms a random coil conformation and thereby can mediate an increased in vivo and/or in vitro stability to the FVIII protein. Since the random coil domain does not adopt a stable structure or function by itself, the biological activity mediated by the FVIII protein is essentially preserved. In other embodiments, the PAS sequences that form random coil domain are biologically inert, especially with respect to proteolysis in blood plasma, immunogenicity, isoelectric point/electrostatic behaviour, binding to cell surface receptors or internalisation, but are still biodegradable, which provides clear advantages over synthetic polymers such as PEG.

Non-limiting examples of the PAS sequences forming random coil conformation comprise an amino acid sequence selected from the group consisting of ASPAAPAPASPAAPAPSAPA (SEQ ID NO: 36), AAPASPAPAAPSAPAPAAPS (SEQ ID NO: 37), APSSPSPSAPSSPSPASPSS (SEQ ID NO: 38), APSSPSPSAPSSPSPASPS (SEQ ID NO: 39), SSPSAPSPSSPASPSPSSPA (SEQ ID NO: 40), AASPAAPSAPPAAASPAAPSAPPA (SEQ ID NO: 41) and ASAAAPAAASAAASAPSAAA (SEQ ID NO: 42) or any combinations thereof. Additional examples of PAS sequences are known from, e.g., US Pat. Publ. No. 2010/0292130 A1 and PCT Appl. Publ. No. WO 2008/155134 A1.

8. HAP Sequence

In certain embodiments, the heterologous moiety is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200.

9. Transferrin or Fragment Thereof

In certain embodiments, the heterologous moiety is transferrin or a fragment thereof. Any transferrin can be used to make the FVIII proteins of the disclosure. As an example, wild-type human TF (TF) is a 679 amino acid protein, of approximately 75 KDa (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and S95936 (www.ncbi.nlm.nih.gov/), all of which are herein incorporated by reference in their entirety. Transferrin comprises two domains, N domain and C domain. N domain comprises two subdomains, N1 domain and N2 domain, and C domain comprises two subdomains, C1 domain and C2 domain.

In one embodiment, the transferrin heterologous moiety includes a transferrin splice variant. In one example, a transferrin splice variant can be a splice variant of human transferrin, e.g., Genbank Accession AAA61140. In another embodiment, the transferrin portion of the chimeric protein includes one or more domains of the transferrin sequence, e.g., N domain, C domain, N1 domain, N2 domain, C1 domain, C2 domain or any combinations thereof.

10. Clearance Receptors

In certain embodiments, the heterologous moiety is a clearance receptor, fragment, variant, or derivative thereof. LRP1 is a 600 kDa integral membrane protein that is implicated in the receptor-mediate clearance of a variety of proteins, such as Factor X. See, e.g., Narita et al., Blood 91:555-560 (1998).

11. Von Willebrand Factor or Fragments Thereof

In certain embodiments, the heterologous moiety is von Willebrand Factor (VWF) or one or more fragments thereof.

VWF (also known as F8VWF) is a large multimeric glycoprotein present in blood plasma and produced constitutively in endothelium (in the Weibel-Palade bodies), megakaryocytes (α-granules of platelets), and subendothelian connective tissue. The basic VWF monomer is a 2813 amino acid protein. Every monomer contains a number of specific domains with a specific function, the D′ and D3 domains (which together bind to Factor VIII), the A1 domain (which binds to platelet GPIb-receptor, heparin, and/or possibly collagen), the A3 domain (which binds to collagen), the C1 domain (in which the RGD domain binds to platelet integrin αIIbβ3 when this is activated), and the “cysteine knot” domain at the C-terminal end of the protein (which VWF shares with platelet-derived growth factor (PDGF), transforming growth factor-β (TGFβ) and β-human chorionic gonadotropin (βHCG)).

The 2813 monomer amino acid sequence for human VWF is reported as Accession Number NP000543.2 in Genbank. The nucleotide sequence encoding the human VWF is reported as Accession Number NM000552.3 in Genbank. SEQ ID NO: 44 (FIG. 11B) is the amino acid sequence encoded by SEQ ID NO: 43. The D′ domain includes amino acids 764 to 866 of SEQ ID NO: 44. The D3 domain includes amino acids 867 to 1240 of SEQ ID NO: 44.

In plasma, 95-98% of FVIII circulates in a tight non-covalent complex with full-length VWF. The formation of this complex is important for the maintenance of appropriate plasma levels of FVIIII in vivo. Lenting et al., Blood. 92(11): 3983-96 (1998); Lenting et al., J. Thromb. Haemost. 5(7): 1353-60 (2007). When FVIII is activated due to proteolysis at positions 372 and 740 in the heavy chain and at position 1689 in the light chain, the VWF bound to FVIII is removed from the activated FVIII.

In certain embodiments, the heterologous moiety is full length von Willebrand Factor. In other embodiments, the heterologous moiety is a von Willebrand Factor fragment. As used herein, the term “VWF fragment” or “VWF fragments” used herein means any VWF fragments that interact with FVIII and retain at least one or more properties that are normally provided to FVIII by full-length VWF, e.g., preventing premature activation to FVIIIa, preventing premature proteolysis, preventing association with phospholipid membranes that could lead to premature clearance, preventing binding to FVIII clearance receptors that can bind naked FVIII but not VWF-bound FVIII, and/or stabilizing the FVIII heavy chain and light chain interactions. In a specific embodiment, the heterologous moiety is a (VWF) fragment comprising a D′ domain and a D3 domain of VWF. The VWF fragment comprising the D′ domain and the D3 domain can further comprise a VWF domain selected from the group consisting of an A1 domain, an A2 domain, an A3 domain, a D1 domain, a D2 domain, a D4 domain, a B1 domain, a B2 domain, a B3 domain, a C1 domain, a C2 domain, a CK domain, one or more fragments thereof, and any combinations thereof. Additional examples of the polypeptide having FVIII activity fused to the VWF fragment are disclosed in U.S. provisional patent application No. 61/667,901, filed Jul. 3, 2012, and U.S. Publication No. 2015/0023959 A1, which are both incorporated herein by reference in its entirety.

12. Linker Moieties

In certain embodiments, the heterologous moiety is a peptide linker.

As used herein, the terms “peptide linkers” or “linker moieties” refer to a peptide or polypeptide sequence (e.g., a synthetic peptide or polypeptide sequence) which connects two domains in a linear amino acid sequence of a polypeptide chain.

In some embodiments, heterologous nucleotide sequences encoding peptide linkers can be inserted between the optimized FVIII polynucleotide sequences of the disclosure and a heterologous nucleotide sequence encoding, for example, one of the heterologous moieties described above, such as albumin. Peptide linkers can provide flexibility to the chimeric polypeptide molecule. Linkers are not typically cleaved, however such cleavage can be desirable. In one embodiment, these linkers are not removed during processing.

A type of linker which can be present in a chimeric protein of the disclosure is a protease cleavable linker which comprises a cleavage site (i.e., a protease cleavage site substrate, e.g., a factor XIa, Xa, or thrombin cleavage site) and which can include additional linkers on either the N-terminal of C-terminal or both sides of the cleavage site. These cleavable linkers when incorporated into a construct of the disclosure result in a chimeric molecule having a heterologous cleavage site.

In one embodiment, an FVIII polypeptide encoded by a nucleic acid molecule of the instant disclosure comprises two or more Fc domains or moieties linked via a cscFc linker to form an Fc region comprised in a single polypeptide chain. The cscFc linker is flanked by at least one intracellular processing site, i.e., a site cleaved by an intracellular enzyme. Cleavage of the polypeptide at the at least one intracellular processing site results in a polypeptide which comprises at least two polypeptide chains.

Other peptide linkers can optionally be used in a construct of the disclosure, e.g., to connect an FVIII protein to an Fc region. Some exemplary linkers that can be used in connection with the disclosure include, e.g., polypeptides comprising GlySer amino acids described in more detail below.

In one embodiment, the peptide linker is synthetic, i.e., non-naturally occurring. In one embodiment, a peptide linker includes peptides (or polypeptides) (which can or cannot be naturally occurring) which comprise an amino acid sequence that links or genetically fuses a first linear sequence of amino acids to a second linear sequence of amino acids to which it is not naturally linked or genetically fused in nature. For example, in one embodiment the peptide linker can comprise non-naturally occurring polypeptides which are modified forms of naturally occurring polypeptides (e.g., comprising a mutation such as an addition, substitution or deletion). In another embodiment, the peptide linker can comprise non-naturally occurring amino acids. In another embodiment, the peptide linker can comprise naturally occurring amino acids occurring in a linear sequence that does not occur in nature. In still another embodiment, the peptide linker can comprise a naturally occurring polypeptide sequence.

For example, in certain embodiments, a peptide linker can be used to fuse identical Fc moieties, thereby forming a homodimeric scFc region. In other embodiments, a peptide linker can be used to fuse different Fc moieties (e.g. a wild-type Fc moiety and an Fc moiety variant), thereby forming a heterodimeric scFc region.

In another embodiment, a peptide linker comprises or consists of a gly-ser linker. In one embodiment, a scFc or cscFc linker comprises at least a portion of an immunoglobulin hinge and a gly-ser linker. As used herein, the term “gly-ser linker” refers to a peptide that consists of glycine and serine residues. In certain embodiments, said gly-ser linker can be inserted between two other sequences of the peptide linker. In other embodiments, a gly-ser linker is attached at one or both ends of another sequence of the peptide linker. In yet other embodiments, two or more gly-ser linker are incorporated in series in a peptide linker. In one embodiment, a peptide linker of the disclosure comprises at least a portion of an upper hinge region (e.g., derived from an IgG1, IgG2, IgG3, or IgG4 molecule), at least a portion of a middle hinge region (e.g., derived from an IgG1, IgG2, IgG3, or IgG4 molecule) and a series of gly/ser amino acid residues.

Peptide linkers of the disclosure are at least one amino acid in length and can be of varying lengths. In one embodiment, a peptide linker of the disclosure is from about 1 to about 50 amino acids in length. As used in this context, the term “about” indicates +/−two amino acid residues. Since linker length must be a positive integer, the length of from about 1 to about 50 amino acids in length, means a length of from 1-3 to 48-52 amino acids in length. In another embodiment, a peptide linker of the disclosure is from about 10 to about 20 amino acids in length. In another embodiment, a peptide linker of the disclosure is from about 15 to about 50 amino acids in length. In another embodiment, a peptide linker of the disclosure is from about 20 to about 45 amino acids in length. In another embodiment, a peptide linker of the disclosure is from about 15 to about 35 or about 20 to about 30 amino acids in length. In another embodiment, a peptide linker of the disclosure is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 1000, or 2000 amino acids in length. In one embodiment, a peptide linker of the disclosure is 20 or 30 amino acids in length.

In some embodiments, the peptide linker can comprise at least two, at least three, at least four, at least five, at least 10, at least 20, at least 30, at least 40, at least 50, at least 60, at least 70, at least 80, at least 90, or at least 100 amino acids. In other embodiments, the peptide linker can comprise at least 200, at least 300, at least 400, at least 500, at least 600, at least 700, at least 800, at least 900, or at least 1,000 amino acids. In some embodiments, the peptide linker can comprise at least about 10, 20, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, or 2000 amino acids. The peptide linker can comprise 1-5 amino acids, 1-10 amino acids, 1-20 amino acids, 10-50 amino acids, 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, or 900-1000 amino acids.

Peptide linkers can be introduced into polypeptide sequences using techniques known in the art. Modifications can be confirmed by DNA sequence analysis. Plasmid DNA can be used to transform host cells for stable production of the polypeptides produced.

### Monomer-Dimer Hybrids

In some embodiments, the isolated nucleic acid molecules of the disclosure which further comprise a heterologous nucleotide sequence encode a monomer-dimer hybrid molecule comprising FVIII.

The term “monomer-dimer hybrid” used herein refers to a chimeric protein comprising a first polypeptide chain and a second polypeptide chain, which are associated with each other by a disulfide bond, wherein the first chain comprises Factor VIII and a first Fc region and the second chain comprises, consists essentially of, or consists of a second Fc region without the FVIII. The monomer-dimer hybrid construct thus is a hybrid comprising a monomer aspect having only one clotting factor and a dimer aspect having two Fc regions.

### Expression Control Element

In some embodiments, the nucleic acid molecule or vector of the disclosure further comprises at least one expression control sequence. A expression control sequences as used herein is any regulatory nucleotide sequence, such as a promoter sequence or promoter-enhancer combination, which facilitates the efficient transcription and translation of the coding nucleic acid to which it is operably linked. For example, the isolated nucleic acid molecule of the disclosure can be operably linked to at least one transcription control sequence. The gene expression control sequence can, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. Other constitutive promoters are known to those of ordinary skill in the art. The promoters useful as gene expression sequences of the disclosure also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In one embodiment, the disclosure includes expression of a transgene under the control of a tissue specific promoter and/or enhancer. In another embodiment, the promoter or other expression control sequence selectively enhances expression of the transgene in liver cells. Examples of liver specific promoters include, but are not limited to, a mouse thyretin promoter (mTTR), an endogenous human factor VIII promoter (F8), human alpha-1-antitrypsin promoter (hAAT), human albumin minimal promoter, and mouse albumin promoter. In a particular embodiment, the promoter comprises a mTTR promoter. The mTTR promoter is described in R. H. Costa et al., 1986, Mol. Cell. Biol. 6:4697. The F8 promoter is described in Figueiredo and Brownlee, 1995, J. Biol. Chem. 270:11828-11838.

Expression levels can be further enhanced to achieve therapeutic efficacy using one or more enhancers. One or more enhancers can be provided either alone or together with one or more promoter elements. Typically, the expression control sequence comprises a plurality of enhancer elements and a tissue specific promoter. In one embodiment, an enhancer comprises one or more copies of the α-1-microglobulin/bikunin enhancer (Rouet et al., 1992, J. Biol. Chem. 267:20765-20773; Rouet et al., 1995, Nucleic Acids Res. 23:395-404; Rouet et al., 1998, Biochem. J. 334:577-584; III et al., 1997, Blood Coagulation Fibrinolysis 8:S23-S30). In another embodiment, an enhancer is derived from liver specific transcription factor binding sites, such as EBP, DBP, HNF1, HNF3, HNF4, HNF6, with Enh1, comprising HNF1, (sense)-HNF3, (sense)-HNF4, (antisense)-HNF1, (antisense)-HNF6, (sense)-EBP, (antisense)-HNF4 (antisense).

In a particular example, a promoter useful for the disclosure comprises SEQ ID NO: 69 (i.e., ET promoter; FIG. 11Y), which is also known as GenBank No. AY661265. See also Vigna et al., Molecular Therapy 11(5):763 (2005). Examples of other suitable vectors and gene regulatory elements are described in WO 02/092134, EP1395293, or U.S. Pat. Nos. 6,808,905, 7,745,179, or 7,179,903, which are incorporated by reference herein in their entireties.

In general, the expression control sequences shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined coding nucleic acid. The gene expression sequences optionally include enhancer sequences or upstream activator sequences as desired.

### Vectors Systems

Some embodiments of the present disclosure are directed to vectors comprising one or more codon optimized nucleic acid molecules encoding a polypeptide with FVIII activity described herein, host cells comprising the vectors, and methods of treating a bleeding disorder using the vectors. The present disclosure meets an important need in the art by providing a vector comprising an optimized FVIII sequence that demonstrates increased expression in a subject and potentially result in greater therapeutic efficacy when used in gene therapy methods.

In some embodiments, the present disclosure provides a vector comprising an isolated nucleic acid molecule comprising a nucleotide sequence which comprises a first nucleic acid sequence encoding an N-terminal portion of a FVII) polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3 or (ii) nucleotides 58-1791 of SEQ ID NO: 4; and wherein the N-terminal portion and the C-terminal portion together have a FVIII polypeptide activity. In other embodiments, the nucleic acid molecule comprises a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5; or (iv) nucleotides 1792-2277 and 2320-4374 of SEQ ID NO: 6; and wherein the N-terminal portion and the C-terminal portion together have a FVIII polypeptide activity.

In some embodiments, the present disclosure provides a vector comprising an isolated nucleic acid molecule comprising a nucleotide sequence encoding a polypeptide with FVIII activity, wherein the nucleotide sequence comprises a nucleic acid sequence having at least about 91%, at least about 92%, at least about 93%, at least about 94%, at least about 95%, at least about 96%, at least about 97%, at least about 98%, or at least about 99% sequence identity to (i) nucleotides 58-4374 of SEQ ID NO: 1 or (ii) nucleotides 58-2277 and 2320-4374 of SEQ ID NO: 1 and is operably linked to a promoter, a target sequence, or both. In other embodiments, the nucleic acid sequence comprises (i) nucleotides 58-4374 of SEQ ID NO: 1 or (ii) nucleotides 58-2277 and 2320-4374 of SEQ ID NO: 1.

In some embodiments, the present disclosure provides a vector comprising an isolated nucleic acid molecule comprising a nucleotide sequence encoding a polypeptide with FVIII activity, wherein the nucleotide sequence comprises a nucleic acid sequence having at least about 94%, at least about 95%, at least about 96%, at least about 97%, at least about 98%, or at least about 99% sequence identity to (i) nucleotides 58-4374 of SEQ ID NO: 2 or (ii) nucleotides 58-2277 and 2320-4374 of SEQ ID NO: 2 and is operably linked to a promoter, a target sequence, or both. In other embodiments, the nucleic acid sequence comprises (i) nucleotides 58-4374 of SEQ ID NO: 2 or (ii) nucleotides 58-2277 and 2320-4374 of SEQ ID NO: 2.

In some embodiments, the present disclosure provides a vector comprising an isolated nucleic acid molecule comprising a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to (i) nucleotides 58-4374 of SEQ ID NO: 70 or (ii) nucleotides 58-2277 and 2320-4374 of SEQ ID NO: 70 and is operably linked to a promoter, a target sequence, or both. In other embodiments, the nucleic acid sequence comprises (i) nucleotides 58-4374 of SEQ ID NO: 70 or (ii) nucleotides 58-2277 and 2320-4374 of SEQ ID NO: 70.

In some embodiments, the present disclosure provides a vector comprising an isolated nucleic acid molecule comprising a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to (i) nucleotides 58-4374 of SEQ ID NO: 71 or (ii) nucleotides 58-2277 and 2320-4374 of SEQ ID NO: 71 and is operably linked to a promoter, a target sequence, or both. In other embodiments, the nucleic acid sequence comprises (i) nucleotides 58-4374 of SEQ ID NO: 71 or (ii) nucleotides 58-2277 and 2320-4374 of SEQ ID NO: 71.

In some embodiments, the present disclosure provides a vector comprising an isolated nucleic acid molecule comprising a nucleotide sequence encoding a polypeptide with FVIII activity, wherein the nucleotide sequence comprises a nucleic acid sequence having at least about 92%, at least about 93%, at least about 94%, at least about 95%, at least about 96%, at least about 97%, at least about 98%, or at least about 99% sequence identity (i) nucleotides 58-4374 of SEQ ID NO: 3 or (ii) nucleotides 58-2277 and 2320-4374 of SEQ ID NO: 3 and is operably linked to a promoter, a target sequence, or both. In other embodiments, the nucleic acid sequence comprises (i) nucleotides 58-4374 of SEQ ID NO: 3 or (ii) nucleotides 58-2277 and 2320-4374 of SEQ ID NO: 3.

In some embodiments, the present disclosure provides a vector comprising an isolated nucleic acid molecule comprising a nucleotide sequence encoding a polypeptide with FVIII activity, wherein the nucleotide sequence comprises a nucleic acid sequence at least about 91%, at least about 92%, at least about 93%, at least about 94%, at least about 95%, at least about 96%, at least about 97%, at least about 98%, or at least about 99% sequence identity to (i) nucleotides 58-4374 of SEQ ID NO: 4 or (ii) nucleotides 58-2277 and 2320-4374 of SEQ ID NO: 4 and is operably linked to a promoter, a target sequence, or both. In other embodiments, the nucleic acid sequence comprises (i) nucleotides 58-4374 of SEQ ID NO: 4 or (ii) nucleotides 58-2277 and 2320-4374 of SEQ ID NO: 4.

In some embodiments, the present disclosure provides a vector comprising an isolated nucleic acid molecule comprising a nucleotide sequence encoding a polypeptide with FVIII activity, wherein the nucleotide sequence comprises a nucleic acid sequence having at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of SEQ ID NO: 5 or (ii) nucleotides 58-2277 and 2320-4374 of SEQ ID NO: 5 and is operably linked to a promoter, a target sequence, or both. In other embodiments, the nucleic acid sequence comprises (i) nucleotides 58-4374 of SEQ ID NO: 5 or (ii) nucleotides 58-2277 and 2320-4374 of SEQ ID NO: 5.

In some embodiments, the present disclosure provides a vector comprising an isolated nucleic acid molecule comprising a nucleotide sequence encoding a polypeptide with FVIII activity, wherein the nucleotide sequence comprises a nucleic acid sequence having at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of SEQ ID NO: 6 or (ii) nucleotides 58-2277 and 2320-4374 of SEQ ID NO: 6 and is operably linked to a promoter, a target sequence, or both. In other embodiments, the nucleic acid sequence comprises (i) nucleotides 58-4374 of SEQ ID NO: 6 or (ii) nucleotides 58-2277 and 2320-4374 of SEQ ID NO: 6.

Suitable vectors for the disclosure include expression vectors, viral vectors, and plasmid vectors. In one embodiment, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

Expression vectors of the disclosure will include optimized polynucleotides encoding the BDD FVIII protein described herein. In one embodiment, the optimized coding sequences for the BDD FVIII protein is operably linked to an expression control sequence. As used herein, two nucleic acid sequences are operably linked when they are covalently linked in such a way as to permit each component nucleic acid sequence to retain its functionality. A coding sequence and a gene expression control sequence are said to be operably linked when they are covalently linked in such a way as to place the expression or transcription and/or translation of the coding sequence under the influence or control of the gene expression control sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding nucleic acid sequence if the gene expression sequence were capable of effecting transcription of that coding nucleic acid sequence such that the resulting transcript is translated into the desired protein or polypeptide.

Viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991).

In one embodiment, the virus is an adeno-associated virus, a double-stranded DNA virus. The adeno-associated virus can be engineered to be replication-deficient and is capable of infecting a wide range of cell types and species. It further has advantages such as heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In another embodiment, the viral vector is an adeno-associated virus (AAV) that has been manipulated to carry a polynucleotide encoding a FVIII protein as disclosed herein. General methods for obtaining recombinant AAVs (rAAVs) have been disclosed. See, for example, U.S. Pat. No. 8,734,809, 2013/0195801 as well as the references cited therein. In some embodiments, a rAAV vector comprises one or more AAV inverted terminal repeats (ITRs) and a transgene of interest (e.g., an optimized FVIII polynucleotide sequence). In certain embodiments, the methods of making rAAV involve culturing a desired host cell which contains a nucleic acid sequence encoding an AAV capsid protein or fragment thereof; a functional rep gene; a rAAV vector composed of, AAV inverted terminal repeats (ITRs) and a transgene of interest; and sufficient helper functions to permit packaging of the recombinant AAV vector into the AAV capsid proteins. Materials and methods for performing these and related procedures have been disclosed, for example, in U.S. Pat. No. 8,734,809, 2013/0195801, PCT/US1997/015692, PCT/US2002/033692, PCT/US2002/033630, WO2007/148971, WO00/20561, WO03/042361, and WO2007/04670.

One or more of different AAV vector sequences derived from nearly any serotype can be used in accord with the present disclosure. Choice of a particular AAV vector sequence will be guided by known parameters such as tropism of interest, required vector yields, etc. Generally, the AAV serotypes have genomic sequences of significant homology at the amino acid and the nucleic acid levels, provide a related set of genetic functions, produce virions which are related, and replicate and assemble similarly. For the genomic sequence of the various AAV serotypes and an overview of the genomic similarities see, e.g., GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 are an illustrative source of AAV nucleotide sequences for use in the context of the present disclosure. AAV6, AAV7, AAV8 or AAV9 or newly developed AAV-like particles obtained by e.g. capsid shuffling techniques and AAV capsid libraries, or from newly designed, developed or evolved ITR's are also suitable for certain disclosure applications. See Dalkara, D et al. (2013), Sci. Transl. Med. 5(189): 189ra76; Kotterman, MA Nat. Rev. Genet. (2014) 15(7):455.

In certain embodiments however, AAV vectors with significant tropism to the liver and related tissues will be of interest for expressing the FVIII proteins disclosed herein. Non-limiting examples include AAV serotypes 1, 2, 6 and 8. See, e.g., Torres-Torranteras et al. (2014) 22: 901 and references cited therein.

In other embodiments, the vector is derived from lentivirus. In certain embodiments, the vector is a vector of a recombinant lentivirus capable of infecting non-dividing cells.

The lentiviral genome and the proviral DNA typically have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA.

However, the resulting mutant remains capable of directing the synthesis of all virion proteins. The disclosure provides a method of producing a recombinant lentivirus capable of infecting a non-dividing cell comprising transfecting a suitable host cell with two or more vectors carrying the packaging functions, namely gag, pol and env, as well as rev and tat. As will be disclosed herein below, vectors lacking a functional tat gene are desirable for certain applications. Thus, for example, a first vector can provide a nucleic acid encoding a viral gag and a viral pol and another vector can provide a nucleic acid encoding a viral env to produce a packaging cell. Introducing a vector providing a heterologous gene, herein identified as a transfer vector, into that packaging cell yields a producer cell which releases infectious viral particles carrying the foreign gene of interest.

According to the above-indicated configuration of vectors and foreign genes, the second vector can provide a nucleic acid encoding a viral envelope (env) gene. The env gene can be derived from nearly any suitable virus, including retroviruses. In some embodiments, the env protein is an amphotropic envelope protein which allows transduction of cells of human and other species.

Examples of retroviral-derived env genes include, but are not limited to: Moloney murine leukemia virus (MoMuLV or MMLV), Harvey murine sarcoma virus (HaMuSV or HSV), murine mammary tumor virus (MuMTV or MMTV), gibbon ape leukemia virus (GaLV or GALV), human immunodeficiency virus (HIV) and Rous sarcoma virus (RSV). Other env genes such as Vesicular stomatitis virus (VSV) protein G (VSV G), that of hepatitis viruses and of influenza also can be used.

The vector providing the viral env nucleic acid sequence is associated operably with regulatory sequences described elsewhere herein.

In certain embodiments, the vector includes a lentiviral vector in which the HIV virulence genes env, vif, vpr, vpu and nef were deleted without compromising the ability of the vector to transduce non-dividing cells.

In some embodiments, the vector includes a lentiviral vector which comprises a deletion of the U3 region of the 3′ LTR. The deletion of the U3 region can be the complete deletion or a partial deletion.

In some embodiments, the lentiviral vector of the disclosure comprising the FVIII nucleotide sequence described herein can be transfected in a cell with (a) a first nucleotide sequence comprising a gag, a pol, or gag and pol genes and (b) a second nucleotide sequence comprising a heterologous env gene; wherein the lentiviral vector lacks a functional tat gene. In other embodiments, the cell is further transfected with a fourth nucleotide sequence comprising a rev gene. In certain embodiments, the lentiviral vector lacks functional genes selected from vif, vpr, vpu, vpx and nef, or a combination thereof.

In certain embodiments, a lentiviral vector comprises one or more nucleotide sequences encoding a gag protein, a Rev-response element, a central polypurine track (cPPT), or any combination thereof.

Examples of the lentiviral vectors are disclosed in WO9931251, WO9712622, WO9817815, WO9817816, and WO9818934, which are incorporated herein by reference in their entireties.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

### Tissue Specific Expression

In certain embodiments, it will be useful to include within the vector one or more miRNA target sequences which, for example, are operably linked to the optimized FVIII transgene. Thus, the disclosure also provides at least one miRNA sequence target operably linked to the optimized FVIII nucleotide sequence or otherwise inserted within a vector. More than one copy of a miRNA target sequence included in the vector can increase the effectiveness of the system. Also included are different miRNA target sequences. For example, vectors which express more than one transgene can have the transgene under control of more than one miRNA target sequence, which can be the same or different. The miRNA target sequences can be in tandem, but other arrangements are also included. The transgene expression cassette, containing miRNA target sequences, can also be inserted within the vector in antisense orientation. Antisense orientation can be useful in the production of viral particles to avoid expression of gene products which can otherwise be toxic to the producer cells. In other embodiments, the vector comprises 1, 2, 3, 4, 5, 6, 7 or 8 copies of the same or different miRNA target sequence. However in certain other embodiments, the vector will not include any miRNA target sequence. Choice of whether or not to include an miRNA target sequence (and how many) will be guided by known parameters such as the intended tissue target, the level of expression required, etc.

In one embodiment, the target sequence is an miR-223 target which has been reported to block expression most effectively in myeloid committed progenitors and at least partially in the more primitive HSPC. miR-223 target can block expression in differentiated myeloid cells including granulocytes, monocytes, macrophages, myeloid dendritic cells. miR-223 target can also be suitable for gene therapy applications relying on robust transgene expression in the lymphoid or erythroid lineage. miR-223 target can also block expression very effectively in human HSC.

In another embodiment, the target sequence is an miR142 target (tccataaagt aggaaacact aca (SEQ ID NO: 43)). In one embodiment, the vector comprises 4 copies of miR-142 target sequences. In certain embodiments, the complementary sequence of hematopoietic-specific microRNAs, such as miR-142 (142T), is incorporated into the 3′ untranslated region of a vector, e.g., lentiviral vectors (LV), making the transgene-encoding transcript susceptible to miRNA-mediated down-regulation. By this method, transgene expression can be prevented in hematopoietic-lineage antigen presenting cells (APC), while being maintained in non-hematopoietic cells (Brown et al., Nat Med 2006). This strategy can imposes a stringent post-transcriptional control on transgene expression and thus enables stable delivery and long-term expression of transgenes. In some embodiments, miR-142 regulation prevents immune-mediated clearance of transduced cells and/or induce antigen-specific Regulatory T cells (T regs) and mediate robust immunological tolerance to the transgene-encoded antigen.

In some embodiments, the target sequence is an miR181 target. Chen C-Z and Lodish H, Seminars in Immunology (2005) 17(2):155-165 discloses miR-181, a miRNA specifically expressed in B cells within mouse bone marrow (Chen and Lodish, 2005). It also discloses that some human miRNAs are linked to leukemias.

The target sequence can be fully or partially complementary to the miRNA. The term “fully complementary” means that the target sequence has a nucleic acid sequence which is 100% complementary to the sequence of the miRNA which recognizes it. The term “partially complementary” means that the target sequence is only in part complementary to the sequence of the miRNA which recognizes it, whereby the partially complementary sequence is still recognized by the miRNA. In other words, a partially complementary target sequence in the context of the present disclosure is effective in recognizing the corresponding miRNA and effecting prevention or reduction of transgene expression in cells expressing that miRNA. Examples of the miRNA target sequences are described at WO2007/000668, WO2004/094642, WO2010/055413, or WO2010/125471, which are incorporated herein by reference in their entireties.

### Host Cells

The disclosure also provides a host cell comprising a nucleic acid molecule or vector of the disclosure. As used herein, the term “transformation” shall be used in a broad sense to refer to the introduction of DNA into a recipient host cell that changes the genotype and consequently results in a change in the recipient cell.

“Host cells” refers to cells that have been transformed with vectors constructed using recombinant DNA techniques and encoding at least one heterologous gene. The host cells of the present disclosure are preferably of mammalian origin; most preferably of human or mouse origin. Those skilled in the art are credited with ability to preferentially determine particular host cell lines which are best suited for their purpose. Exemplary host cell lines include, but are not limited to, CHO, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte), PER.C6®, NS0, CAP, BHK21, and HEK 293 (human kidney). In one particular embodiment, the host cell is selected from the group consisting of: a CHO cell, a HEK293 cell, a BHK21 cell, a PER.C6© cell, a NS0 cell, and a CAP cell. Host cell lines are typically available from commercial services, the American Tissue Culture Collection, or from published literature.

Introduction of the isolated nucleic acid molecules or vectors of the disclosure into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or flourescence-activated cell sorter analysis (FACS), immunohistochemistry and the like.

Host cells comprising the isolated nucleic acid molecules or vectors of the disclosure are grown in an appropriate growth medium. As used herein, the term “appropriate growth medium” means a medium containing nutrients required for the growth of cells. Nutrients required for cell growth can include a carbon source, a nitrogen source, essential amino acids, vitamins, minerals, and growth factors. Optionally, the media can contain one or more selection factors. Optionally the media can contain bovine calf serum or fetal calf serum (FCS). In one embodiment, the media contains substantially no IgG. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g., MEM, DMEM, DMEM/F12). In one embodiment, the medium is CDoptiCHO (Invitrogen, Carlsbad, CA.). In another embodiment, the medium is CD17 (Invitrogen, Carlsbad, CA.). Selection of a medium appropriate for the particular cell line used is within the level of those ordinary skilled in the art.

### Preparation of Polypeptides

The disclosure also provides a polypeptide encoded by a nucleic acid molecule of the disclosure. In other embodiments, the polypeptide of the disclosure is encoded by a vector comprising the isolated nucleic molecules of the disclosure. In yet other embodiments, the polypeptide of the disclosure is produced by a host cell comprising the isolated nucleic molecules of the disclosure.

In other embodiments, the disclosure also provides a method of producing a polypeptide with FVIII activity, comprising culturing a host cell of the disclosure under conditions whereby a polypeptide with FVIII activity is produced, and recovering the polypeptide with FVIII activity. In some embodiments, the expression of the polypeptide with FVIII activity is increased relative to a host cell cultured under the same conditions but comprising a reference nucleotide sequence comprising SEQ ID NO: 16, the parental FVIII gene sequence.

In other embodiments, the disclosure provides a method of increasing the expression of a polypeptide with FVIII activity comprising culturing a host cell of the disclosure under conditions whereby a polypeptide with FVIII activity is expressed by the nucleic acid molecule, wherein the expression of the polypeptide with FVIII activity is increased relative to a host cell cultured under the same conditions comprising a reference nucleic acid molecule comprising SEQ ID NO: 16.

In other embodiments, the disclosure provides a method of improving yield of a polypeptide with FVIII activity comprising culturing a host cell under conditions whereby a polypeptide with FVIII activity is produced by the nucleic acid molecule, wherein the yield of polypeptide with FVIII activity is increased relative to a host cell cultured under the same conditions comprising a reference nucleic acid sequence comprising SEQ ID NO: 16.

In other embodiments, the disclosure provides a method of improving yield of a polypeptide with FVIII activity comprising culturing a host cell comprising a nucleotide sequence encoding the polypeptide, wherein the codon adaptation index of a 3′ portion of the nucleotide sequence is increased relative to a 5′ portion of the nucleotide sequence; wherein the yield of the polypeptide with FVIII activity is increased relative to a host cell cultured under the same conditions comprising a reference nucleic acid sequence comprising SEQ ID NO: 16. In some embodiments, the codon adaptation index of the 5′ portion of the nucleotide sequence is increased, decreased, or unchanged relative to the codon optimization index of SEQ ID NO: 16.

In other embodiments, the disclosure provides a method of improving yield of a polypeptide with FVIII activity comprising culturing a host cell comprising a nucleotide sequence encoding the polypeptide, wherein the codon adaptation index of a 5′ portion of the nucleotide sequence is increased relative to a 3′ portion of the nucleotide sequence; wherein the yield of polypeptide with FVIII activity is increased relative to a host cell cultured under the same conditions comprising a reference nucleic acid sequence comprising SEQ ID NO: 16. In some embodiments, the codon adaptation index of the 3′ portion of the nucleotide sequence is increased, decreased, or unchanged relative to the codon optimization index of SEQ ID NO: 16.

In other embodiments, the disclosure provides a method of improving yield of a polypeptide with FVIII activity comprising culturing a host cell comprising a nucleotide sequence encoding the polypeptide, wherein the codon adaptation index of a portion of the nucleotide encoding a C-terminal portion of the polypeptide is increased relative to a portion of the nucleotide encoding an N-terminal portion of the polypeptide; wherein the yield of polypeptide with FVIII activity is increased relative to a host cell cultured under the same conditions comprising a reference nucleic acid sequence comprising SEQ ID NO: 16. In some embodiments, the codon adaptation index of the portion of the nucleotide encoding the N-terminal portion of the polypeptide is increased, decreased, or unchanged relative to the codon optimization index of SEQ ID NO: 16.

In other embodiments, the disclosure provides a method of improving yield of a polypeptide with FVIII activity comprising culturing a host cell comprising a nucleotide sequence encoding the polypeptide, wherein the codon adaptation index of a portion of the nucleotide encoding a N-terminal portion of the polypeptide is increased relative to a portion of the nucleotide encoding an C-terminal portion of the polypeptide; wherein the yield of polypeptide with FVIII activity is increased relative to a host cell cultured under the same conditions comprising a reference nucleic acid sequence comprising SEQ ID NO: 16. In some embodiments, the codon adaptation index of the portion of the nucleotide encoding the C-terminal portion of the polypeptide is increased, decreased, or unchanged relative to the codon optimization index of SEQ ID NO: 16.

In certain embodiments of improving yield of a polypeptide with FVIII activity, the 5′ portion of the nucleotide sequence, when properly aligned, corresponds with about nucleotides 1-1791 of SEQ ID NO: 1, nucleotides 58-1791 of SEQ ID NO: 1, or a fragment thereof. In other embodiments, a polypeptide encoded by the 5′ portion of the nucleotide, when properly aligned, corresponds with about amino acids 1-497 of SEQ ID NO: 17, amino acids 20-497 of SEQ ID NO: 17, or a fragment thereof. In certain embodiments, the portion of the nucleotide sequence encoding the N-terminal portion of the polypeptide, when properly aligned, corresponds with about nucleotides 1-1791 of SEQ ID NO: 1, nucleotides 58-1791 of SEQ ID NO: 1, or a fragment thereof. In some embodiments, the 3′ portion of the nucleotide sequence, when properly aligned, corresponds with about nucleotides 1792-4374 of SEQ ID NO: 1 or a fragment thereof. In other embodiments, a polypeptide encoded by the 3′ portion of the nucleotide, when properly aligned, corresponds with about amino acids 498-1458 of SEQ ID NO: 17, or a fragment thereof. In certain embodiments, the portion of the nucleotide sequence encoding the C-terminal portion of the polypeptide, when properly aligned, corresponds with about nucleotides 1792-4374 of SEQ ID NO: 1 or a fragment thereof

In some embodiments, the expression of the FVIII polypeptide is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 50-fold, at least about 60-fold, at least about 70-fold, at least about 80-fold, at least about 90-fold, at least about 100-fold, at least about 150-fold, or at least about 200-fold relative to a host cell cultured under the same conditions comprising a reference nucleic acid sequence comprising SEQ ID NO: 16.

A variety of methods are available for recombinantly producing a FVIII protein from the optimized nucleic acid molecule of the disclosure. A polynucleotide of the desired sequence can be produced by de novo solid-phase DNA synthesis or by PCR mutagenesis of an earlier prepared polynucleotide. Oligonucleotide-mediated mutagenesis is one method for preparing a substitution, insertion, deletion, or alteration (e.g., altered codon) in a nucleotide sequence. For example, the starting DNA is altered by hybridizing an oligonucleotide encoding the desired mutation to a single-stranded DNA template. After hybridization, a DNA polymerase is used to synthesize an entire second complementary strand of the template that incorporates the oligonucleotide primer. In one embodiment, genetic engineering, e.g., primer-based PCR mutagenesis, is sufficient to incorporate an alteration, as defined herein, for producing a polynucleotide of the disclosure.

For recombinant protein production, an optimized polynucleotide sequence of the disclosure encoding the FVIII protein is inserted into an appropriate expression vehicle, i.e., a vector which contains the necessary elements for the transcription and translation of the inserted coding sequence, or in the case of an RNA viral vector, the necessary elements for replication and translation.

The polynucleotide sequence of the disclosure is inserted into the vector in proper reading frame. The expression vector is then transfected into a suitable target cell which will express the polypeptide. Transfection techniques known in the art include, but are not limited to, calcium phosphate precipitation (Wigler et al. 1978, Cell 14: 725) and electroporation (Neumann et al. 1982, EMBO, J. 1: 841). A variety of host-expression vector systems can be utilized to express the FVIII proteins described herein in eukaryotic cells. In one embodiment, the eukaryotic cell is an animal cell, including mammalian cells (e.g. HEK293 cells, PER.C6®, CHO, BHK, Cos, HeLa cells). A polynucleotide sequence of the disclosure can also code for a signal sequence that will permit the FVIII protein to be secreted. One skilled in the art will understand that while the FVIII protein is translated the signal sequence is cleaved by the cell to form the mature protein. Various signal sequences are known in the art, e.g., native factor VII signal sequence, native factor IX signal sequence and the mouse IgK light chain signal sequence. Alternatively, where a signal sequence is not included the FVIII protein can be recovered by lysing the cells.

The FVIII protein of the disclosure can be synthesized in a transgenic animal, such as a rodent, goat, sheep, pig, or cow. The term “transgenic animals” refers to non-human animals that have incorporated a foreign gene into their genome. Because this gene is present in germline tissues, it is passed from parent to offspring. Exogenous genes are introduced into single-celled embryos (Brinster et al. 1985, Proc. Natl. Acad. Sci. USA 82:4438). Methods of producing transgenic animals are known in the art including transgenics that produce immunoglobulin molecules (Wagner et al. 1981, Proc. Natl. Acad. Sci. USA 78: 6376; McKnight et al. 1983, Cell 34: 335; Brinster et al. 1983, Nature 306: 332; Ritchie et al. 1984, Nature 312: 517; Baldassarre et al. 2003, Theriogenology 59: 831; Robl et al. 2003, Theriogenology 59: 107; Malassagne et al. 2003, Xenotransplantation 10 (3): 267).

The expression vectors can encode for tags that permit for easy purification or identification of the recombinantly produced protein. Examples include, but are not limited to, vector pUR278 (Ruther et al. 1983, EMBO J. 2: 1791) in which the FVIII protein described herein coding sequence can be ligated into the vector in frame with the lac Z coding region so that a hybrid protein is produced; pGEX vectors can be used to express proteins with a glutathione S-transferase (GST) tag. These proteins are usually soluble and can easily be purified from cells by adsorption to glutathione-agarose beads followed by elution in the presence of free glutathione. The vectors include cleavage sites (e.g., PreCission Protease (Pharmacia, Peapack, N. J.)) for easy removal of the tag after purification.

For the purposes of this disclosure, numerous expression vector systems can be employed. These expression vectors are typically replicable in the host organisms either as episomes or as an integral part of the host chromosomal DNA. Expression vectors can include expression control sequences including, but not limited to, promoters (e.g., naturally-associated or heterologous promoters), enhancers, signal sequences, splice signals, enhancer elements, and transcription termination sequences. Preferably, the expression control sequences are eukaryotic promoter systems in vectors capable of transforming or transfecting eukaryotic host cells. Expression vectors can also utilize DNA elements which are derived from animal viruses such as bovine papilloma virus, polyoma virus, adenovirus, vaccinia virus, baculovirus, retroviruses (RSV, MMTV or MOMLV), cytomegalovirus (CMV), or SV40 virus. Others involve the use of polycistronic systems with internal ribosome binding sites.

Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362). Cells which have integrated the DNA into their chromosomes can be selected by introducing one or more markers which allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation.

An example of a vector useful for expressing an optimized FVIII sequence is NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in cells, followed by selection in G418 containing medium and methotrexate amplification. Vector systems are also taught in U.S. Pat. Nos. 5,736,137 and 5,658,570, each of which is incorporated by reference in its entirety herein. This system provides for high expression levels, e.g., >30 μg/cell/day. Other exemplary vector systems are disclosed e.g., in U.S. Pat. No. 6,413,777.

In other embodiments the polypeptides of the disclosure of the instant disclosure can be expressed using polycistronic constructs. In these expression systems, multiple gene products of interest such as multiple polypeptides of multimer binding protein can be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980 which is also incorporated herein.

More generally, once the vector or DNA sequence encoding a polypeptide has been prepared, the expression vector can be introduced into an appropriate host cell. That is, the host cells can be transformed. Introduction of the plasmid into the host cell can be accomplished by various techniques well known to those of skill in the art, as discussed above. The transformed cells are grown under conditions appropriate to the production of the FVIII polypeptide, and assayed for FVIII polypeptide synthesis. Exemplary assay techniques include enzyme-linked immunosorbent assay (ELISA), radioimmunoassay (RIA), or fluorescence-activated cell sorter analysis (FACS), immunohistochemistry and the like.

In descriptions of processes for isolation of polypeptides from recombinant hosts, the terms “cell” and “cell culture” are used interchangeably to denote the source of polypeptide unless it is clearly specified otherwise. In other words, recovery of polypeptide from the “cells” can mean either from spun down whole cells, or from the cell culture containing both the medium and the suspended cells.

The host cell line used for protein expression is preferably of mammalian origin; most preferably of human or mouse origin, as the isolated nucleic acids of the disclosure have been optimized for expression in human cells. Exemplary host cell lines have been described above. In one embodiment of the method to produce a polypeptide with FVIII activity, the host cell is a HEK293 cell. In another embodiment of the method to produce a polypeptide with FVIII activity, the host cell is a CHO cell.

Genes encoding the polypeptides of the disclosure can also be expressed in non-mammalian cells such as bacteria or yeast or plant cells. In this regard it will be appreciated that various unicellular non-mammalian microorganisms such as bacteria can also be transformed; i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules.

Alternatively, optimized nucleotide sequences of the disclosure can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for polypeptides in operable linkage with a promoter and enhancer from a mammary gland specific gene, such as casein or beta lactoglobulin.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein. An affinity tag sequence (e.g. a His(6) tag) can optionally be attached or included within the polypeptide sequence to facilitate downstream purification.

Once expressed, the FVIII protein can be purified according to standard procedures of the art, including ammonium sulfate precipitation, affinity column chromatography, HPLC purification, gel electrophoresis and the like (see generally Scopes, Protein Purification (Springer-Verlag, N.Y., (1982)). Substantially pure proteins of at least about 90 to 95% homogeneity are preferred, and 98 to 99% or more homogeneity most preferred, for pharmaceutical uses.

### Pharmaceutical Composition

Compositions containing an isolated nucleic acid molecule, a polypeptide having FVIII activity encoded by the nucleic acid molecule, a vector, or a host cell of the present disclosure can contain a suitable pharmaceutically acceptable carrier. For example, they can contain excipients and/or auxiliaries that facilitate processing of the active compounds into preparations designed for delivery to the site of action.

The pharmaceutical composition can be formulated for parenteral administration (i.e. intravenous, subcutaneous, or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

Suitable formulations for parenteral administration also include aqueous solutions of the active compounds in water-soluble form, for example, water-soluble salts. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides. Aqueous injection suspensions can contain substances, which increase the viscosity of the suspension, including, for example, sodium carboxymethyl cellulose, sorbitol and dextran. Optionally, the suspension can also contain stabilizers. Liposomes also can be used to encapsulate the molecules of the disclosure for delivery into cells or interstitial spaces. Exemplary pharmaceutically acceptable carriers are physiologically compatible solvents, dispersion media, coatings, antibacterial and antifungal agents, isotonic and absorption delaying agents, water, saline, phosphate buffered saline, dextrose, glycerol, ethanol and the like. In some embodiments, the composition comprises isotonic agents, for example, sugars, polyalcohols such as mannitol, sorbitol, or sodium chloride. In other embodiments, the compositions comprise pharmaceutically acceptable substances such as wetting agents or minor amounts of auxiliary substances such as wetting or emulsifying agents, preservatives or buffers, which enhance the shelf life or effectiveness of the active ingredients.

Compositions of the disclosure can be in a variety of forms, including, for example, liquid (e.g., injectable and infusible solutions), dispersions, suspensions, semi-solid and solid dosage forms. The preferred form depends on the mode of administration and therapeutic application.

The composition can be formulated as a solution, micro emulsion, dispersion, liposome, or other ordered structure suitable to high drug concentration. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The active ingredient can be formulated with a controlled-release formulation or device. Examples of such formulations and devices include implants, transdermal patches, and microencapsulated delivery systems. Biodegradable, biocompatible polymers can be used, for example, ethylene vinyl acetate, polyanhydrides, polyglycolic acid, collagen, polyorthoesters, and polylactic acid. Methods for the preparation of such formulations and devices are known in the art. See, e.g., Sustained and Controlled Release Drug Delivery Systems, J. R. Robinson, ed., Marcel Dekker, Inc., New York, 1978.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

Supplementary active compounds can be incorporated into the compositions. In one embodiment, the chimeric protein of the disclosure is formulated with another clotting factor, or a variant, fragment, analogue, or derivative thereof. For example, the clotting factor includes, but is not limited to, factor V, factor VII, factor VIII, factor IX, factor X, factor XI, factor XII, factor XIII, prothrombin, fibrinogen, von Willebrand factor or recombinant soluble tissue factor (rsTF) or activated forms of any of the preceding. The clotting factor of hemostatic agent can also include anti-fibrinolytic drugs, e.g., epsilon-amino-caproic acid, tranexamic acid.

Dosage regimens can be adjusted to provide the optimum desired response. For example, a single bolus can be administered, several divided doses can be administered over time, or the dose can be proportionally reduced or increased as indicated by the exigencies of the therapeutic situation. It is advantageous to formulate parenteral compositions in dosage unit form for ease of administration and uniformity of dosage. See, e.g., Remington's Pharmaceutical Sciences (Mack Pub. Co., Easton, Pa. 1980).

In addition to the active compound, the liquid dosage form can contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

Non-limiting examples of suitable pharmaceutical carriers are also described in Remington's Pharmaceutical Sciences by E. W. Martin. Some examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal administration, the composition can take the form of tablets or lozenges according to conventional protocols.

For administration by inhalation, the compounds for use according to the present disclosure are conveniently delivered in the form of a nebulized aerosol with or without excipients or in the form of an aerosol spray from a pressurized pack or nebulizer, with optionally a propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can also be formulated for rectal administration as a suppository or retention enema, e.g., containing conventional suppository bases such as cocoa butter or other glycerides.

In one embodiment, a pharmaceutical composition comprises a polypeptide having Factor VIII activity, an optimized nucleic acid molecule encoding the polypeptide having Factor VIII activity, the vector comprising the nucleic acid molecule, or the host cell comprising the vector, and a pharmaceutically acceptable carrier. In some embodiments, the composition is administered by a route selected from the group consisting of topical administration, intraocular administration, parenteral administration, intrathecal administration, subdural administration and oral administration. The parenteral administration can be intravenous or subcutaneous administration.

In other embodiments, the composition is used to treat a bleeding disease or condition in a subject in need thereof. The bleeding disease or condition is selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath and any combinations thereof. In still other embodiments, the subject is scheduled to undergo a surgery. In yet other embodiments, the treatment is prophylactic or on-demand.

### Methods of Treatment

The disclosure provides a method of treating a bleeding disorder comprising administering to a subject in need thereof a nucleic acid molecule, vector, or polypeptide of the disclosure. In some embodiments, the bleeding disorder is characterized by a deficiency in FVIII. In some embodiments, the bleeding disorder is hemophilia. In some embodiments, the bleeding disorder is hemophilia A. In some embodiments of the method of treating a bleeding disorder, plasma FVIII activity at 24 hours post administration is increased relative to a subject administered a reference nucleic acid molecule comprising SEQ ID NO: 16, a vector comprising the reference nucleic acid molecule, or a polypeptide encoded by the reference nucleic acid molecule.

In some embodiments, plasma FVIII activity is increased at about 6 hours, at about 12 hours, at about 18 hours, at about 24 hours, at about 36 hours, at about 48 hours, at about 3 days, at about 4 days, at about 5 days, at about 6 days, at about 7 days, at about 8 days, at about 9 days, at about 10 days, at about 11 days, at about 12 days, at about 13 days, at about 14 days, at about 15 days, at about 16 days, at about 17 days, at about 18 days, at about 19 days, at about 20 days, at about 21 days, at about 22 days, at about 23 days, at about 24 days, at about 25 days, at about 26 days, at about 27 days, or at about 28 days post administration relative to a subject administered a reference nucleic acid molecule comprising SEQ ID NO: 16, a viral vector comprising the reference nucleic acid molecule, or a polypeptide encoded by the reference nucleic acid molecule. In certain embodiments, plasma FVIII activity is increased at about 24 hours post administration relative to a subject administered a reference nucleic acid molecule comprising SEQ ID NO: 16, a viral vector comprising the reference nucleic acid molecule, or a polypeptide encoded by the reference nucleic acid molecule. In another embodiment, plasma FVIII activity is increased at about 21 days post administration relative to a subject administered a reference nucleic acid molecule comprising SEQ ID NO: 16, a viral vector comprising the reference nucleic acid molecule, or a polypeptide encoded by the reference nucleic acid molecule.

In some embodiments, plasma FVIII activity post administration is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 50-fold, at least about 60-fold, at least about 70-fold, at least about 80-fold, at least about 90-fold, at least about 100-fold, at least about 150-fold, at least about 200-fold, at least about 250-fold, at least about 300-fold, at least about 350-fold, at least about 400-fold, at least about 450-fold, or at least about 500-fold relative to a subject administered a reference nucleic acid molecule comprising SEQ ID NO: 16, a viral vector comprising the reference nucleic acid molecule, or a polypeptide encoded by the reference nucleic acid molecule. In some embodiments, plasma FVIII activity post administration is increased by at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1500%, at least about 2000%, at least about 2500%, at least about 3000%, at least about 3500%, at least about 4000%, at least about 4500%, at least about 5000%, at least about 5500%, at least about 6000%, at least about 7000%, at least about 8000%, at least about 9000%, at least about 10,000% relative to physiologically normal circulating FVIII levels. In one embodiment, the plasma FVIII activity post administration is increased by at least about 3000 to about 5000% relative to physiologically normal circulating FVIII levels. In some embodiments, at 24 hours post administration of a plasmid comprising a codon-optimized gene encoding polypeptides with Factor VIII (FVIII) activity described herein, or at 21 days post administration of a lentiviral or AAV vector comprising a codon-optimized gene encoding polypeptides with Factor VIII (FVIII) activity described herein, plasma FVIII activity is increased by at least about 6-fold relative to a subject administered a reference nucleic acid molecule comprising SEQ ID NO: 16, a vector comprising the reference nucleic acid molecule, or a polypeptide encoded by the reference nucleic acid molecule. In some embodiments, at 24 hours post administration of a plasmid comprising a codon-optimized gene encoding polypeptides with Factor VIII (FVIII) activity described herein, or at 21 days post administration of a lentiviral or AAV vector comprising a codon-optimized gene encoding polypeptides with Factor VIII (FVIII) activity described herein, plasma FVIII activity is increased by at least about 10-fold relative to a subject administered a reference nucleic acid molecule comprising SEQ ID NO: 16, a vector comprising the reference nucleic acid molecule, or a polypeptide encoded by the reference nucleic acid molecule. In some embodiments, at 24 hours post administration of a plasmid comprising a codon-optimized gene encoding polypeptides with Factor VIII (FVIII) activity described herein, or at 21 days post administration of a lentiviral or AAV vector comprising a codon-optimized gene encoding polypeptides with Factor VIII (FVIII) activity described herein, plasma FVIII activity is increased by at least about 23-fold relative to a subject administered a reference nucleic acid molecule comprising SEQ ID NO: 16, a vector comprising the reference nucleic acid molecule, or a polypeptide encoded by the reference nucleic acid molecule. In some embodiments, at 24 hours post administration of a plasmid comprising a codon-optimized gene encoding polypeptides with Factor VIII (FVIII) activity described herein, or at 21 days post administration of a lentiviral or AAV vector comprising a codon-optimized gene encoding polypeptides with Factor VIII (FVIII) activity described herein, plasma FVIII activity is increased by at least about 18-fold relative to a subject administered a reference nucleic acid molecule comprising SEQ ID NO: 16, a vector comprising the reference nucleic acid molecule, or a polypeptide encoded by the reference nucleic acid molecule. In some embodiments, at 24 hours post administration of a plasmid comprising a codon-optimized gene encoding polypeptides with Factor VIII (FVIII) activity described herein, or at 21 days post administration of a lentiviral or AAV vector comprising a codon-optimized gene encoding polypeptides with Factor VIII (FVIII) activity described herein, plasma FVIII activity is increased by at least about 30-fold relative to a subject administered a reference nucleic acid molecule comprising SEQ ID NO: 16, a vector comprising the reference nucleic acid molecule, or a polypeptide encoded by the reference nucleic acid molecule. In some embodiments, at 24 hours post administration of a plasmid comprising a codon-optimized gene encoding polypeptides with Factor VIII (FVIII) activity described herein, or at 21 days post administration of a lentiviral or AAV vector comprising a codon-optimized gene encoding polypeptides with Factor VIII (FVIII) activity described herein, plasma FVIII activity is increased by at least about 50-fold relative to a subject administered a reference nucleic acid molecule comprising SEQ ID NO: 16, a vector comprising the reference nucleic acid molecule, or a polypeptide encoded by the reference nucleic acid molecule. In some embodiments, at 24 hours post administration of a plasmid comprising a codon-optimized gene encoding polypeptides with Factor VIII (FVIII) activity described herein, or at 21 days post administration of a lentiviral or AAV vector comprising a codon-optimized gene encoding polypeptides with Factor VIII (FVIII) activity described herein, plasma FVIII activity is increased by at least about 100-fold relative to a subject administered a reference nucleic acid molecule comprising SEQ ID NO: 16, a vector comprising the reference nucleic acid molecule, or a polypeptide encoded by the reference nucleic acid molecule.

The disclosure also relates to a method of treating, ameliorating, or preventing a hemostatic disorder in a subject comprising administering a therapeutically effective amount of an isolated nucleic acid molecule of the disclosure or a polypeptide having FVIII activity encoded by the nucleic acid molecule of the disclosure. The treatment, amelioration, and prevention by the isolated nucleic acid molecule or the encoded polypeptide can be a bypass therapy. The subject receiving bypass therapy can have already developed an inhibitor to a clotting factor, e.g., FVIII, or is subject to developing a clotting factor inhibitor.

The nucleic acid molecules, vectors, or polypeptides of the disclosure treat or prevent a hemostatic disorder by promoting the formation of a fibrin clot. The polypeptide having FVIII activity encoded by the nucleic acid molecule of the disclosure can activate a member of a coagulation cascade. The clotting factor can be a participant in the extrinsic pathway, the intrinsic pathway or both.

The nucleic acid molecules, vectors, or polypeptides of the disclosure can be used to treat hemostatic disorders known to be treatable with FVIII. The hemostatic disorders that can be treated using methods of the disclosure include, but are not limited to, hemophilia A, hemophilia B, von Willebrand's disease, Factor XI deficiency (PTA deficiency), Factor XII deficiency, as well as deficiencies or structural abnormalities in fibrinogen, prothrombin, Factor V, Factor VII, Factor X, or Factor XIII, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath. Compositions for administration to a subject include nucleic acid molecules which comprise an optimized nucleotide sequence of the disclosure encoding a FVIII clotting factor (for gene therapy applications) as well as FVIII polypeptide molecules.

In some embodiments, the hemostatic disorder is an inherited disorder. In one embodiment, the subject has hemophilia A. In other embodiments, the hemostatic disorder is the result of a deficiency in FVIII. In other embodiments, the hemostatic disorder can be the result of a defective FVIII clotting factor.

In another embodiment, the hemostatic disorder can be an acquired disorder. The acquired disorder can result from an underlying secondary disease or condition. The unrelated condition can be, as an example, but not as a limitation, cancer, an autoimmune disease, or pregnancy. The acquired disorder can result from old age or from medication to treat an underlying secondary disorder (e.g., cancer chemotherapy).

The disclosure also relates to methods of treating a subject that does not have a hemostatic disorder or a secondary disease or condition resulting in acquisition of a hemostatic disorder. The disclosure thus relates to a method of treating a subject in need of a general hemostatic agent comprising administering a therapeutically effective amount of the isolated nucleic acid molecule, vector, or FVIII polypeptide of the disclosure. For example, in one embodiment, the subject in need of a general hemostatic agent is undergoing, or is about to undergo, surgery. The isolated nucleic acid molecule, vector, or FVIII polypeptide of the disclosure can be administered prior to or after surgery as a prophylactic. The isolated nucleic acid molecule, vector, or FVIII polypeptide of the disclosure can be administered during or after surgery to control an acute bleeding episode. The surgery can include, but is not limited to, liver transplantation, liver resection, or stem cell transplantation.

In another embodiment, the isolated nucleic acid molecule, vector, or FVIII polypeptide of the disclosure can be used to treat a subject having an acute bleeding episode who does not have a hemostatic disorder. The acute bleeding episode can result from severe trauma, e.g., surgery, an automobile accident, wound, laceration gun shot, or any other traumatic event resulting in uncontrolled bleeding.

The isolated nucleic acid molecule, vector, or FVIII protein can be used to prophylactically treat a subject with a hemostatic disorder. The isolated nucleic acid molecule, vector, or FVIII protein can be used to treat an acute bleeding episode in a subject with a hemostatic disorder.

In another embodiment, expression of FVIII protein by administering the isolated nucleic acid molecule or vector of the disclosure does not induce an immune response in a subject. In some embodiments, the immune response comprises development of antibodies against FVIII. In some embodiments, the immune response comprises cytokine secretion. In some embodiments, the immune response comprises activation of B cells, T cells, or both B cells and T cells. In some embodiments, the immune response is an inhibitory immune response, wherein the immune response in the subject reduces the activity of the FVIII protein relative to the activity of the FVIII in a subject that has not developed an immune response. In certain embodiments, expression of FVIII protein by administering the isolated nucleic acid molecule or vector, of the disclosure prevents an inhibitory immune response against the FVIII protein or the FVIII protein expressed from the isolated nucleic acid molecule or the vector.

In some embodiments, an isolated nucleic acid molecule, vector, or FVIII protein composition of the disclosure is administered in combination with at least one other agent that promotes hemostasis. Said other agent that promotes hemostasis in a therapeutic with demonstrated clotting activity. As an example, but not as a limitation, the hemostatic agent can include Factor V, Factor VII, Factor IX, Factor X, Factor XI, Factor XII, Factor XIII, prothrombin, or fibrinogen or activated forms of any of the preceding. The clotting factor or hemostatic agent can also include anti-fibrinolytic drugs, e.g., epsilon-amino-caproic acid, tranexamic acid.

In one embodiment of the disclosure, the composition (e.g., the isolated nucleic acid molecule, vector, or FVIII polypeptide) is one in which the FVIII is present in activatable form when administered to a subject. Such an activatable molecule can be activated in vivo at the site of clotting after administration to a subject.

The isolated nucleic acid molecule, vector, or FVIII polypeptide can be administered intravenously, subcutaneously, intramuscularly, or via any mucosal surface, e.g., orally, sublingually, buccally, sublingually, nasally, rectally, vaginally or via pulmonary route. The FVIII protein can be implanted within or linked to a biopolymer solid support that allows for the slow release of the chimeric protein to the desired site.

For oral administration, the pharmaceutical composition can take the form of tablets or capsules prepared by conventional means. The composition can also be prepared as a liquid for example a syrup or a suspension. The liquid can include suspending agents (e.g. sorbitol syrup, cellulose derivatives or hydrogenated edible fats), emulsifying agents (lecithin or acacia), non-aqueous vehicles (e.g. almond oil, oily esters, ethyl alcohol, or fractionated vegetable oils), and preservatives (e.g. methyl or propyl-p-hydroxybenzoates or sorbic acid). The preparations can also include flavoring, coloring and sweetening agents. Alternatively, the composition can be presented as a dry product for constitution with water or another suitable vehicle.

For buccal and sublingual administration the composition can take the form of tablets, lozenges or fast dissolving films according to conventional protocols.

For administration by inhalation, the polypeptide having FVIII activity for use according to the present disclosure are conveniently delivered in the form of an aerosol spray from a pressurized pack or nebulizer (e.g., in PBS), with a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In one embodiment, the route of administration of the isolated nucleic acid molecule, vector, or FVIII polypeptide is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the disclosure, a form for administration would be a solution for injection, in particular for intravenous or intraarterial injection or drip. Usually, a suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, the isolated nucleic acid molecule, vector, or FVIII polypeptide can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject disclosure, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture will preferably have labels or package inserts indicating that the associated compositions are useful for treating a subject suffering from, or predisposed to clotting disorders.

The pharmaceutical composition can also be formulated for rectal administration as a suppository or retention enema, e.g., containing conventional suppository bases such as cocoa butter or other glycerides.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

Dosages for administering a plasmid comprising a codon-optimized gene encoding polypeptides with Factor VIII (FVIII) activity, as described herein, or a polypeptide encoded by the codon-optimized gene can range from 1000 ug/kg to 0.1 ng/kg body weight. In one embodiment, the dosing range is 1 ug/kg to 100 ug/kg. Dosages for administering a lentiviral vector comprising a codon-optimized gene encoding polypeptides with Factor VIII (FVIII) activity, as described herein, can range from 10³ to 10¹⁵ TU/kg. Dosages for administering an AAV vector comprising a codon-optimized gene encoding polypeptides with Factor VIII (FVIII) activity, as described herein, can range from 10⁵ to 10¹⁸ VG/kg.

The isolated nucleic acid molecule, plasmid, vector, or FVIII polypeptide can be administered as a single dose or as multiple doses, wherein the multiple doses can be administered continuously or at specific timed intervals. In vitro assays can be employed to determine optimal dose ranges and/or schedules for administration. In vitro assays that measure clotting factor activity are known in the art. Additionally, effective doses can be extrapolated from dose-response curves obtained from animal models, e.g., a hemophiliac dog (Mount et al. 2002, Blood 99 (8): 2670).

Doses intermediate in the above ranges are also intended to be within the scope of the disclosure. Subjects can be administered such doses daily, on alternative days, weekly or according to any other schedule determined by empirical analysis. An exemplary treatment entails administration in multiple dosages over a prolonged period, for example, of at least six months. In some methods, two or more polypeptides can be administered simultaneously, in which case the dosage of each polypeptide administered falls within the ranges indicated.

The isolated nucleic acid molecule, vector, or FVIII polypeptides of the disclosure can be administered on multiple occasions. Intervals between single dosages can be daily, weekly, monthly or yearly. Intervals can also be irregular as indicated by measuring blood levels of modified polypeptide or antigen in the patient. Alternatively, polypeptides can be administered as a sustained release formulation, in which case less frequent administration is required. Dosage and frequency vary depending on the half-life of the polypeptide or polynucleotide in the patient.

The dosage and frequency of administration can vary depending on whether the treatment is prophylactic or therapeutic. In prophylactic applications, compositions containing the isolated nucleic acid molecule, vector, or FVIII polypeptide or a cocktail thereof are administered to a patient not already in the disease state to enhance the patient's resistance or minimize effects of disease. Such an amount is defined to be a “prophylactic effective dose.” A relatively low dosage is administered at relatively infrequent intervals over a long period of time. Some patients continue to receive treatment for the rest of their lives.

The isolated nucleic acid molecule, vector, or FVIII polypeptides of the disclosure can optionally be administered in combination with other agents that are effective in treating the disorder or condition in need of treatment (e.g., prophylactic or therapeutic).

As used herein, the administration of isolated nucleic acid molecules, vectors, or FVIII polypeptides of the disclosure in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the isolated nucleic acid molecule, vector, or FVIII polypeptide of the instant disclosure can be used in conjunction or combination with an agent or agents (e.g., to provide a combined therapeutic regimen). Exemplary agents with which a polypeptide or polynucleotide of the disclosure can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the polynucleotides or polypeptides of the instant disclosure can vary by subject or can be administered according to what is known in the art. See, e.g., Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996). In another embodiment, an amount of such an agent consistent with the standard of care is administered.

As previously discussed, the polynucleotides and polypeptides of the present disclosure, can be administered in a pharmaceutically effective amount for the in vivo treatment of clotting disorders. In this regard, it will be appreciated that the polypeptides or polynucleotides of the disclosure can be formulated to facilitate administration and promote stability of the active agent. Preferably, pharmaceutical compositions in accordance with the present disclosure comprise a pharmaceutically acceptable, non-toxic, sterile carrier such as physiological saline, non-toxic buffers, preservatives and the like. Of course, the pharmaceutical compositions of the present disclosure can be administered in single or multiple doses to provide for a pharmaceutically effective amount of the polypeptide.

A number of tests are available to assess the function of the coagulation system: activated partial thromboplastin time (aPTT) test, chromogenic assay, ROTEM® assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphosholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g, ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII or FIX. It is used in conjunction with prothrombin time (PT), which measures the extrinsic pathway.

ROTEM® analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

### Gene Therapy

The disclosure provides a method of increasing expression of a polypeptide with FVIII activity in a subject comprising administering the isolated nucleic acid molecule of the disclosure to a subject in need thereof, wherein the expression of the polypeptide is increased relative to a reference nucleic acid molecule comprising SEQ ID NO: 16. The disclosure also provides a method of increasing expression of a polypeptide with FVIII activity in a subject comprising administering a vector of the disclosure to a subject in need thereof, wherein the expression of the polypeptide is increased relative to a vector comprising a reference nucleic acid molecule.

Somatic gene therapy has been explored as a possible treatment for hemophilia A. Gene therapy is a particularly appealing treatment for hemophilia because of its potential to cure the disease through continuous endogenous production of FVIII following a single administration of vector. Haemophilia A is well suited for a gene replacement approach because its clinical manifestations are entirely attributable to the lack of a single gene product (FVIII) that circulates in minute amounts (200 ng/ml) in the plasma.

A FVIII protein of the disclosure can be produced in vivo in a mammal, e.g., a human patient, using a gene therapy approach to treatment of a bleeding disease or disorder selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath would be therapeutically beneficial. In one embodiment, the bleeding disease or disorder is hemophilia. In another embodiment, the bleeding disease or disorder is hemophilia A. This involves administration of an optimized FVIII encoding nucleic acid operably linked to suitable expression control sequences. In certain embodiment, these sequences are incorporated into a viral vector. Suitable viral vectors for such gene therapy include adenoviral vectors, lentiviral vectors, baculoviral vectors, Epstein Barr viral vectors, papovaviral vectors, vaccinia viral vectors, herpes simplex viral vectors, and adeno associated virus (AAV) vectors. The viral vector can be a replication-defective viral vector. In other embodiments, an adenoviral vector has a deletion in its E1 gene or E3 gene. In other embodiments, the sequences are incorporated into a non-viral vector known to those skilled in the art.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

Having generally described this disclosure, a further understanding can be obtained by reference to the examples provided herein. These examples are for purposes of illustration only and are not intended to be limiting.

### EXAMPLES

### Example 1. Codon Optimization Strategy

Eight codon optimized BDD FVIII variants were created by controlling the codon usage bias, including coFVIII-3 (SEQ ID NO: 1; FIG. 1A), coFVIII-4 (SEQ ID NO: 2; FIG. 1B), coFVIII-5 (SEQ ID NO: 70; FIG. 1C), coFVIII-6 (SEQ ID NO: 71; FIG. 1D), coFVIII-52 (SEQ ID NO: 3; FIG. 1E), coFVIII-62 (SEQ ID NO: 4; FIG. 1F), coFVIII-25 (SEQ ID NO: 5; FIG. 1G), and coFVIII-26 (SEQ ID NO: 6; FIG. 1H). The online tool Eugene was used to facilitate codon optimization as previously described (See Gaspar et al., “EuGene: maximizing synthetic gene design for heterologous expression,” Bioinformatics 28:2683-84 (2012)), and several codon usage parameters, such as codon adaptation index (CAI) and relative synonymous codon usage (RSCU), were monitored (Table 5). All variants were adjusted to CAI≥83% and RSCU≥1.63, while the parental B-domain deleted FVIII sequence, prior to optimization, has a CAI of 74% and an RSCU of 1.12 (Table 5).

TABLE 5
Codon Optimization Parameters

| | Parental | | | | | coFVIII- | coFVIII- | coFVIII- | coFVIII- |
|---|---|---|---|---|---|---|---|---|---|
| | BDD FVIII | coFVIII-3 | coFVIII-4 | coFVIII-5 | coFVIII-6 | 52 | 62 | 25 | 26 |
| Codon | 74 | 91 | 97 | 83 | 83 | 91 | 91 | 88 | 88 |
| Adaptation | | | | | | | | | |
| Index (CAI; %) | | | | | | | | | |
| Frequency of | 39 | 65 | 92 | 64 | 64 | 79 | 79 | 74 | 75 |
| Optimal | | | | | | | | | |
| Codons (FOP) | | | | | | | | | |
| GC Content (%) | 44.10 | 52.10 | 60.80 | 55.7 | 55.9% | 58.30 | 58.30 | 57.30 | 57.60 |
| Relative | 1.12 | 2.32 | 2.72 | 1.63 | 1.63 | 2.22 | 2.19 | 2.04 | 2.58 |
| Synonymous | | | | | | | | | |
| Codon Usage | | | | | | | | | |
| (RSCU) | | | | | | | | | |
| Codon Pair | 0.19 | 0.43 | 0.04 | 0.11 | 0.11 | 0.27 | 0.27 | 0.23 | 0.48 |
| Bias | | | | | | | | | |
| Effective | 54.2 | 25.6 | 22.8 | 39.7 | 39.1 | 30.9 | 31.4 | 34.1 | 26.7 |
| number of | | | | | | | | | |
| codons | | | | | | | | | |

In addition to the overall increase of the CAI, the eight variants were designed into three classes based on the distribution of CAI across the coding region, as illustrated in FIG. 2, relative to the non-optimized BDD FVIII sequence (FIG. 2A). The first class comprises BDD FVIII variants with an even distribution of the high CAI across the entire coding region (see FIGS. 2C-2F). The first class includes coFVIII-3 (FIG. 2C), coFVIII-4 (FIG. 2D), coFVIII-5 (FIG. 2E), coFVIII-6 (FIG. 2F), as well as the previously described coFVIII-1 (see International Publication No. WO 2014/127215 (SEQ ID NO: 1)) (FIG. 2B). The second class comprises BDD-FVIII variants with a lower CAI at the N-terminal half of the coding sequence and a higher CAI at the C-terminal half of the coding sequence (see FIGS. 2G and 2H). The second class includes coFVIII-52 (FIG. 2G) and coFVIII-62 (FIG. 2H). The third class comprises BDD FVIII variants with a higher CAI at the N-terminal half of the coding sequence and a lower CAI at the C-terminal half of the coding sequence (see FIGS. 2I and 2J). The third class includes coFVIII-25 (FIG. 2I) and coFVIII-26 (FIG. 2J).

Without being bound by any theory, it was speculated that a higher CAI might correlate with faster protein translation, and these three classes might represent different rates of protein synthesis from the start to finish. For example, translation of a region having a lower CAI might proceed slowly relative to translation of a region having a higher CAI. If so, translation of, e.g., the N-terminal half of coFVIII-52 of coFVIII-62, having a lower CAI, might initially proceed slowly followed by more rapid translation of the C-terminal half, having a higher CAI. This could be preferred for protein folding and post-translational modification during translation without slowing down the overall protein synthesis. The opposite effect might be seen for the coFVIII-25 and coFVIII-26 variants, which have a higher CAI at the N-terminal half and a lower CAI at the C-terminal half.

To ensure the stability of the mRNA, all the FVIII codon optimized variants were adjusted to avoid a number of sites, including cryptic splicing sites, premature polyA sites, RNA instability motifs (ARE), and repeat sequences, and to adjust the GC content (see Table 2).

### Example 2. Cloning and Expression of coFVIII Variants from a pcDNA3 Plasmid

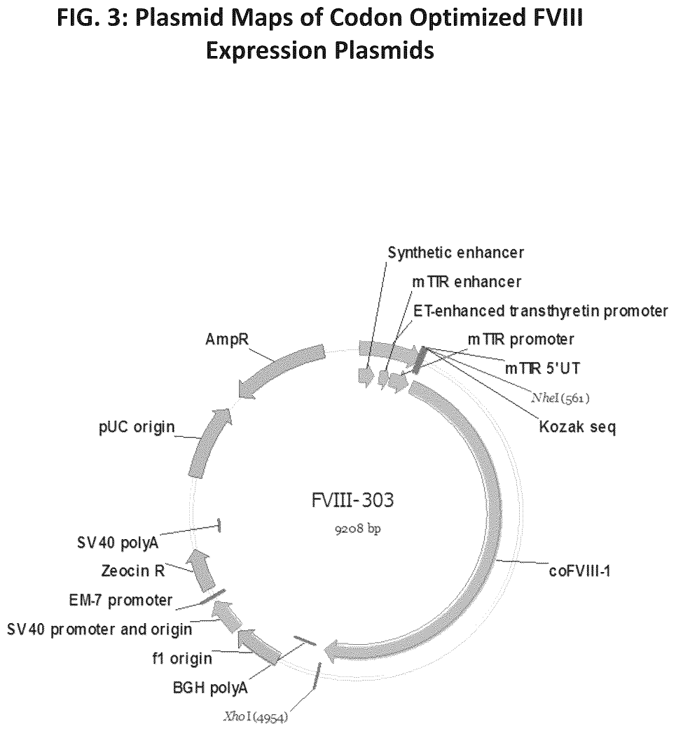

Expression plasmids containing the various FVIII variants were designed for in vivo expression. The non-optimized BDD FVIII (FIG. 1I; SEQ ID NO: 16) and coFVIII-1 (FIG. 11Z; SEQ ID NO: 68) polynucleotides were cloned into a pcDNA3 backbone (Invitrogen), wherein the CMV promoter was replaced by an ET promoter (see FIG. 3). The resulting plasmids, FVIII-311 (BDD FVIII) and FVIII-303 (coFVIII-1), drive the expression of non-optimized BDD FVIII and coFVIII-1, respectively.

In vivo expression of FVIII-311 and FVIII-303 was evaluated in Hem A mice by hydrodynamic injection of 5 μg DNA/mouse of FVIII-303 or FVIII-311. Plasma samples were collected at 24, 48, and 72 hours post-injection, and FVIII activity was determined by a FVIII specific chromogenic assay.

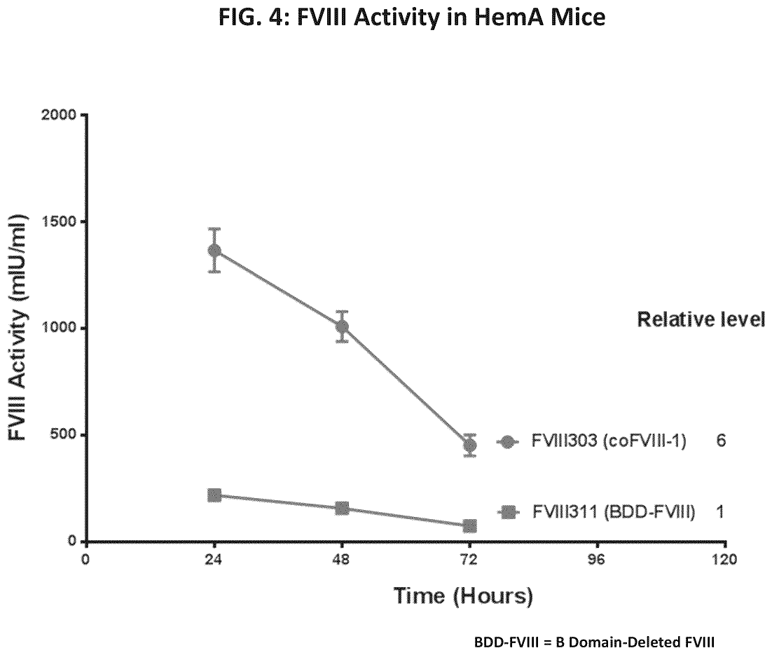

As shown in FIG. 4, the plasma FVIII activity of mice treated with FVIII-311 (BDD FVIII; squares) was 74±43 mU/mL at 72 hours post-injection, whereas the plasma FVIII activity of mice treated with FVIII-303 (coFVIII-1; circles) was 452±170 mU/mL at 72 hours post-injection (FIG. 4). This represents an approximately six-fold increase in the expression of coFVIII-1 relative to non-optimized BDD FVIII.

### Example 3. Cloning and Expressing coFVIII Variants Using a Lentiviral Vector System

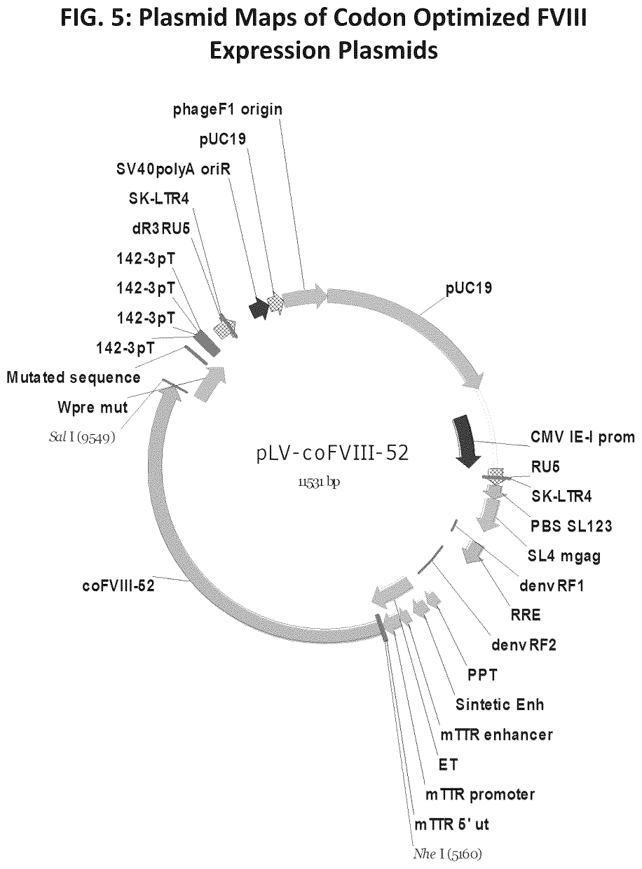

To further assess the expression level of the codon optimized BDD FVIII variants, the coding sequences were cloned into lentiviral plasmids under the control of an ET promoter (see Amendola et al., “Coordinate dual-gene transgenesis by lentiviral vectors carrying synthetic bidirectional promoters,” Nature Biol. 23:108-16 (2005); International Publication No. WO 2000/066759 A1). A plasmid map of pLV-coFVIII-52 is shown in FIG. 5; and plasmids containing non-optimized BDD FVIII (LV-2116), coFVIII-1 (LV-coFVIII-1), coFVIII-3 (LV-coFVIII-3), coFVIII-4 (LV-coFVIII-4), coFVIII-5 (LV-coFVIII-5), and coFVIII-6 (LV-coFVIII-6), coFVIII-62 (LV-coFVIII-62), coFVIII-25 (LV-coFVIII-25), and coFVIII-26 (LV-coFVIII-26) were constructed in the same manner, except that the coFVIII-52 fragment was replaced by each indicated coding sequence using the NheI and SaII sites (Table 6).

TABLE 6
Expression Plasmids Coding for FVIII Variants

| Plasmid ID | Description |
|---|---|
| FVIII-303 | coFVIII-1 under ET promoter in pcDNA3 |
| (coFVIII-1) | |
| FVIII-311 | Parental BDD-FVIII under ET promoter in pcDNA3 |
| (BDD FVIII) | |
| LV-2116 | Parental BDD-FVIII under ET promoter in lentiviral |
| (BDD FVIII) | plasmid |
| LV-coFVIII-1 | coFVIII-1 under ET promoter in lentiviral plasmid |
| LV-coFVIII-3 | coFVIII-3 under ET promoter in lentiviral plasmid |
| LV-coFVIII-4 | coFVIII-4 under ET promoter in lentiviral plasmid |
| LV-coFVIII-5 | coFVIII-5 under ET promoter in lentiviral plasmid |
| LV-coFVIII-6 | coFVIII-6 under ET promoter in lentiviral plasmid |
| LV-coFVIII-52 | coFVIII-52 under ET promoter in lentiviral plasmid |
| LV-coFVIII-62 | coFVIII-62 under ET promoter in lentiviral plasmid |
| LV-coFVIII-25 | coFVIII-25 under ET promoter in lentiviral plasmid |
| LV-coFVIII-26 | coFVIII-26 under ET promoter in lentiviral plasmid |

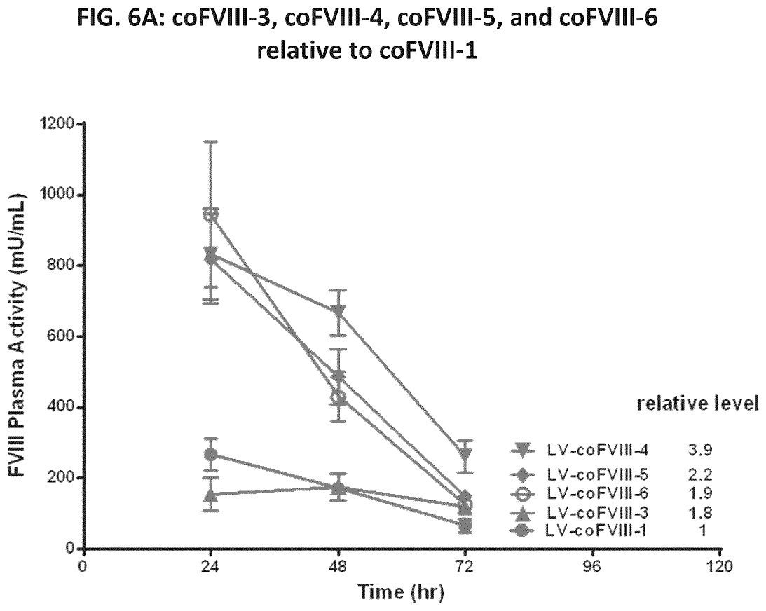

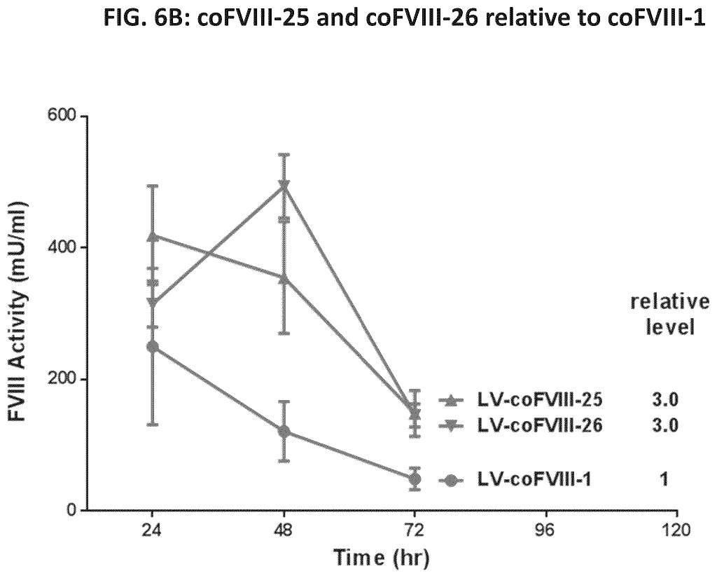

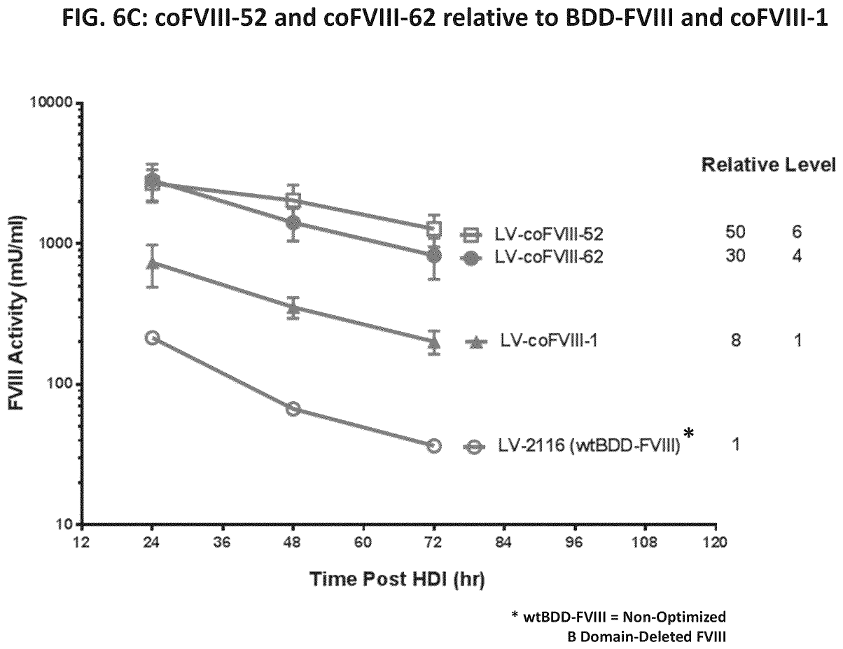

The lentiviral codon optimized FVIII variants were evaluated in HemA mice by hydrodynamic injection at a dose of 5 μg DNA/mouse (FIGS. 6A, 6B) or 20 μg DNA/mouse (FIG. 6C). As shown in FIG. 6, each of coFVIII-3 (FIG. 6A; triangles), coFVIII-4 (FIG. 6A; inverted triangles), coFVIII-5 (FIG. 6A; diamonds), coFVIII-6 (FIG. 6A; open circles), coFVIII-25 (FIG. 6B; triangles), coFVIII-26 (FIG. 6B; inverted triangles), coFVIII-52 (FIG. 6C; squares), and coFVIII-62 (FIG. 6C; filled circles) exhibited higher FVIII activity than coFVIII-1 (FIG. 6A, circles; FIG. 6B, circles; and FIG. 6C, triangles). In particular, coFVIII-25 and coFVIII-26 exhibited a similar expression level at 72 hours post-injection, reaching about 3-fold higher activity than that of the coFVIII-1 (FIG. 6B), which translates into 24-fold higher FVIII activity compared to the non-optimized, parental BDD FVIII (see FIG. 4). Both coFVIII-52 (squares) and coFVIII-62 (filled circles) achieved even higher expression at 72 hours post-injection, exhibiting 6-fold and 4-fold greater expression, respectively, than coFVIII-1 (triangles), and 50-fold and 30-fold greater expression, respectively, than non-optimized, parental BDD FVIII (open circles) (FIG. 6C). These data indicate that the combination of a lower CAI at the N-terminal half of the coding sequence and a higher CAI at the C-terminal half of the coding sequence might be more beneficial for FVIII expression as compared to the reversed distribution of CAI.

### Example 4: Long-Term Lentiviral Expression of Codon-Optimized FVIII Variants in HemA Mice

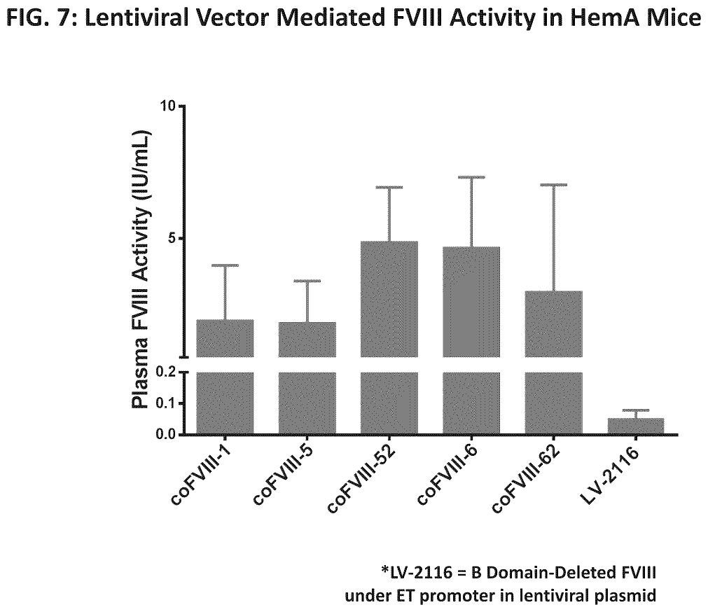

Variants identified to drive high expression of FVIII in HemA mice at 72 hours post-hydrodynamic injection were evaluated for long term FVIII expression by lentiviral vectors mediated gene transfer. Lentiviral vectors were produced in 293T cells by transient transfection and concentrated by ultracentrifugation to about 5E9 TU/ml. The lentiviral vectors were then administered into 12-14 day old HemA mice by retro-orbital injection at a dose of 1E8 TU/mouse. At 21 days after lentiviral injection, the average plasma FVIII activity was about 0.04 IU/ml for mice injected with LV-2116 (BDD FVIII; FIG. 7). Each of coFVIII-1, coFVIII-5, coFVIII-52, coFVIII-6, and coFVIII-62 resulted in a higher circulating FVIII level at 21 days post-injection relative to the LV-2116 (non-optimized B domain deleted FVIII) control. In particular, coFVIII-1 and coFVIII-5 injection yielded a FVIII plasma activity levels of about 1.8 IU/mL, coFVIII-52 yielded a FVIII plasma activity level of about 4.9 IU/mL, coFVIII-6 yielded a FVIII plasma activity levels of about 4.6 IU/mL, and coFVIII-62 yielded a FVIII plasma activity level of about 2.5 IU/mL at 21 days post injection (FIG. 7). The FVIII plasma levels observed in mice injected with LV-coFVIII-6 and LV-coFVIII-52, 4.6 IU/ml and 4.9 IU/ml, respectively, are more than 100-fold higher than the plasma levels observed in mice injected with the LV-2116 (non-optimized BDD-FVIII) control.

### Example 5. coFVIII-XTEN Fusion Constructs

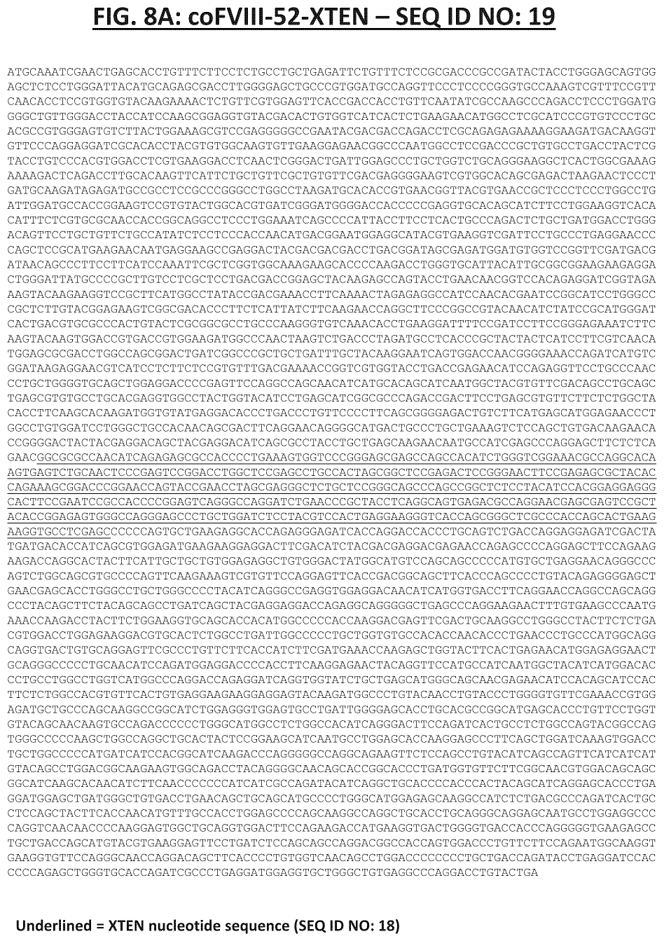

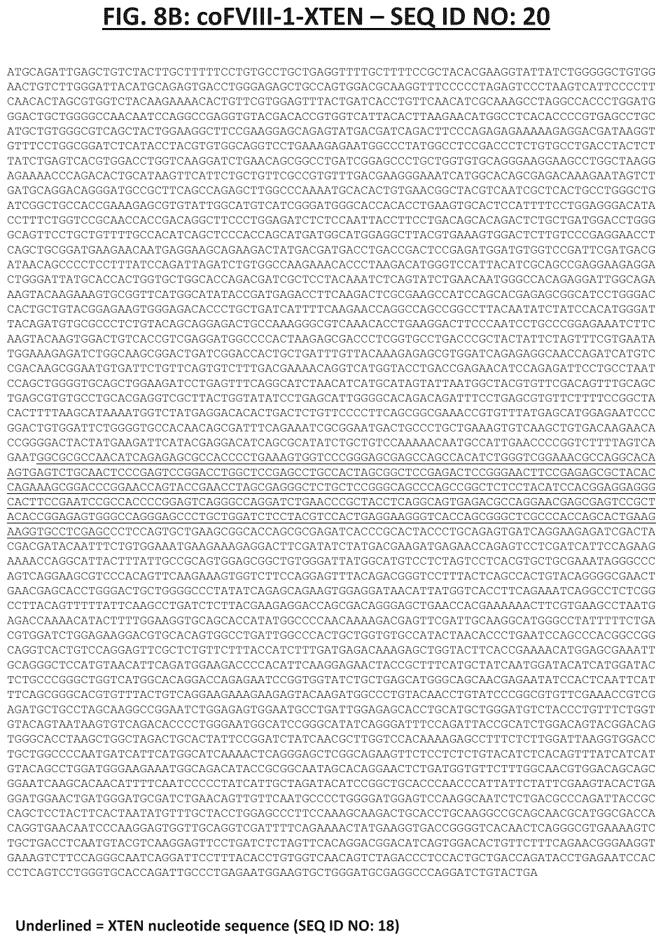

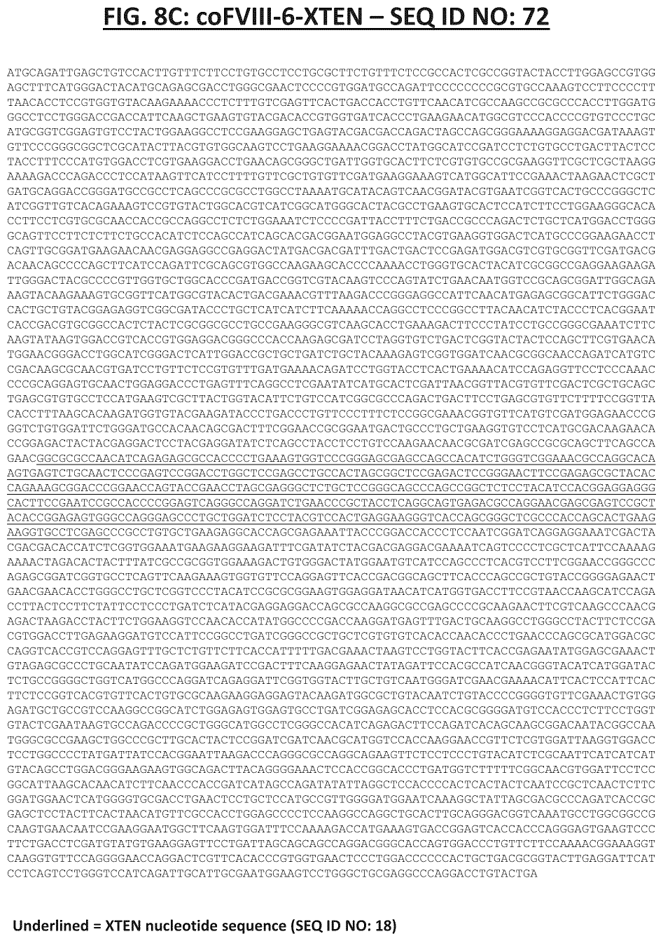

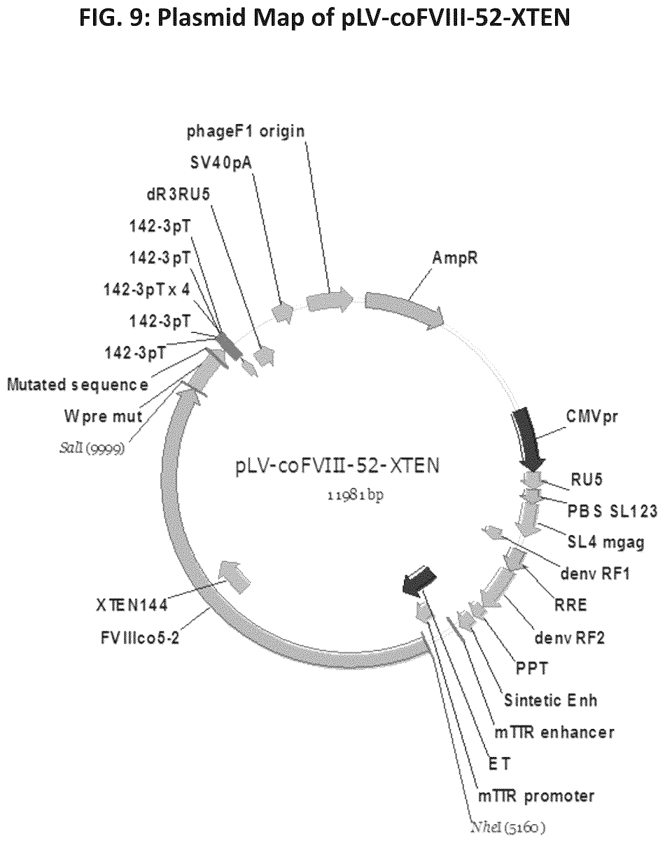

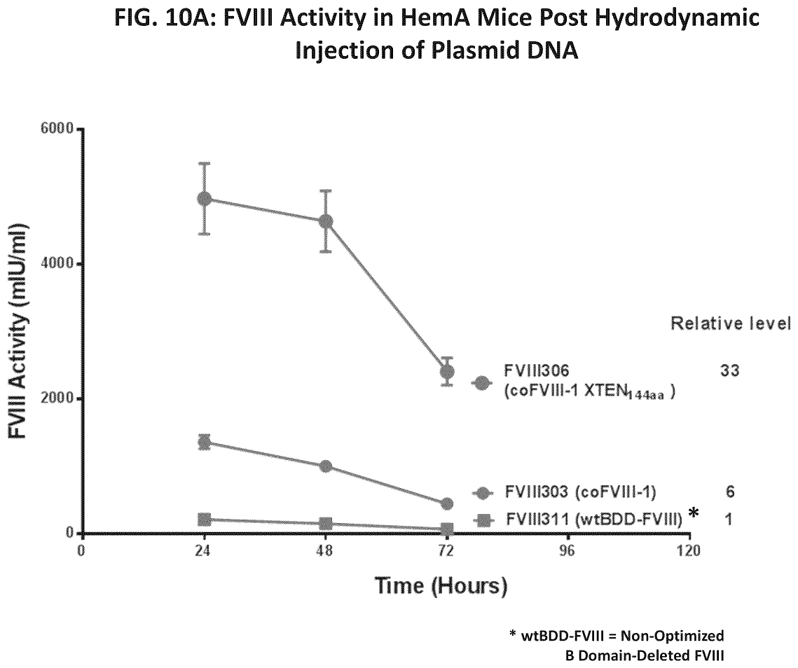

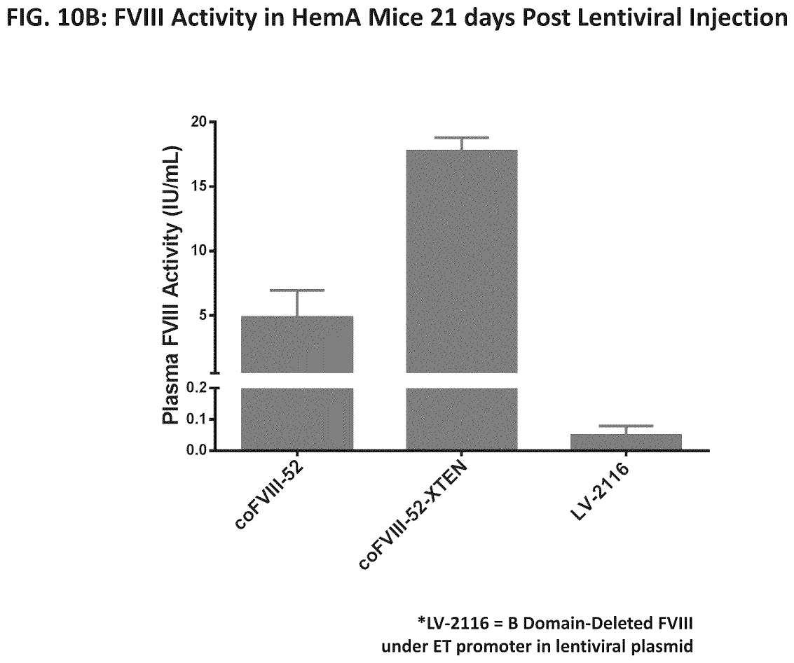

The ability of XTEN to improve the steady state FVIII expression was tested. First, the coding sequence for an XTEN of 144 amino acids (“XTEN₁₄₄”; SEQ ID NO: 18) was inserted at nucleotide 1193 (or after the first 764 amino acids of the encoded polypeptide) of coFVIII-52 and coFVIII-1 to generate coFVIII-52-XTEN (FIG. 8A; SEQ ID NO: 19) and coFVIII-1-XTEN (FIG. 8B; SEQ ID NO: 20), respectively. The coFVIII-1-XTEN sequence was then cloned into a pcDNA3 backbone (Invitrogen) under the control of an ET promoter, as described above, to create the FVIII-306 expression plasmid; and the coFVIII-52-XTEN sequence was cloned into a lentiviral plasmid under the control of an ET promoter, as disclosed above, to create the pLV-coFVIII-52-XTEN (FIG. 9). FVIII-306 (coFVIII-1-XTEN) was administered to HemA mice at 5 μg DNA/mouse by hydrodynamic injection. As compared to FVIII-303 (coFVIII-1; FIG. 10A, small circles) and FVIII-311 (BDD FVIII; FIG. 10A, squares), fusion of XTEN₁₄₄ to coFVIII-1 (FVIII-306; FIG. 10A, large circles) resulted in about 5-fold and 33-fold higher FVIII expression, respectively, in HemA mice at 72 hours post-injection. The effect of XTEN insertion on FVIII expression was also evaluated using lentiviral vector in HemA mice (FIG. 10B). LV-coFVIII-52-XTEN was administered to 12-14 day old HemA mice at 1E8 TU/mouse by retro orbital injection. As compared to LV-coFVIII52 and LV-2116 (BDD-FVIII), fusion of XTEN₁₄₄ to coFVIII-52 (FIG. 10B) resulted in about 4-fold and 450-fold higher FVIII expression, respectively, in HemA mice at 21 days post-injection.

Lentiviral vectors comprising each of coFVIII-3, co-FVIII-4, coFVIII-5, coFVIII-6, coFVIII-62, coFVIII-25, and coFVIII-26 fused to XTEN₁₄₄ and fused to an ET promoter will be made as described above. The vectors will be tested for their expression of FVIII proteins.

### Example 6. Expression of coFVIII Constructs

Codon optimized FVIII variants were cloned into lentiviral plasmids, as illustrated in FIG. 9, by standard molecular cloning techniques. Lentiviral vectors were then produced in HEK293 cells through transient transfection and isolated by ultracentrifugation.

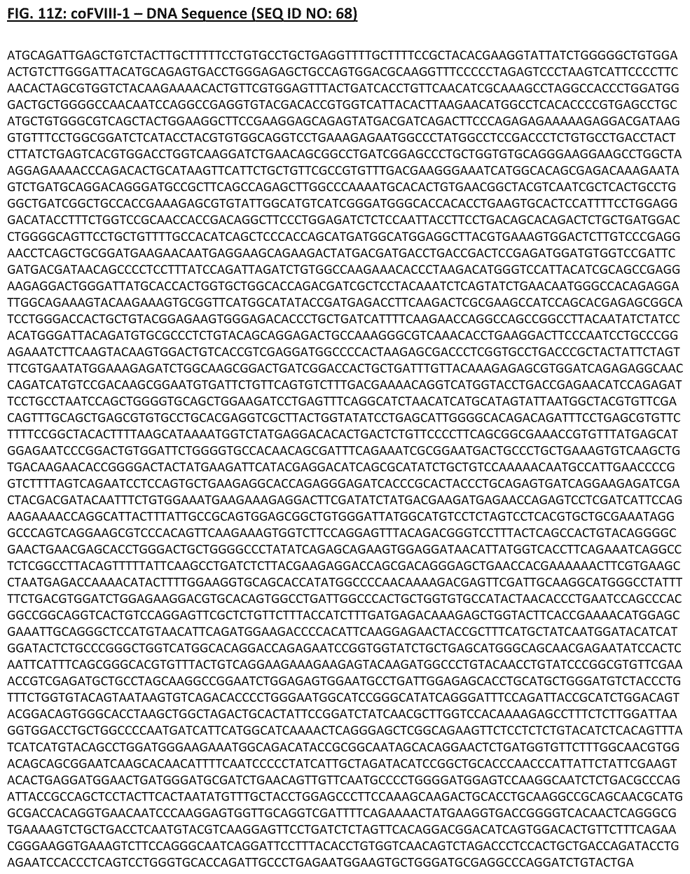

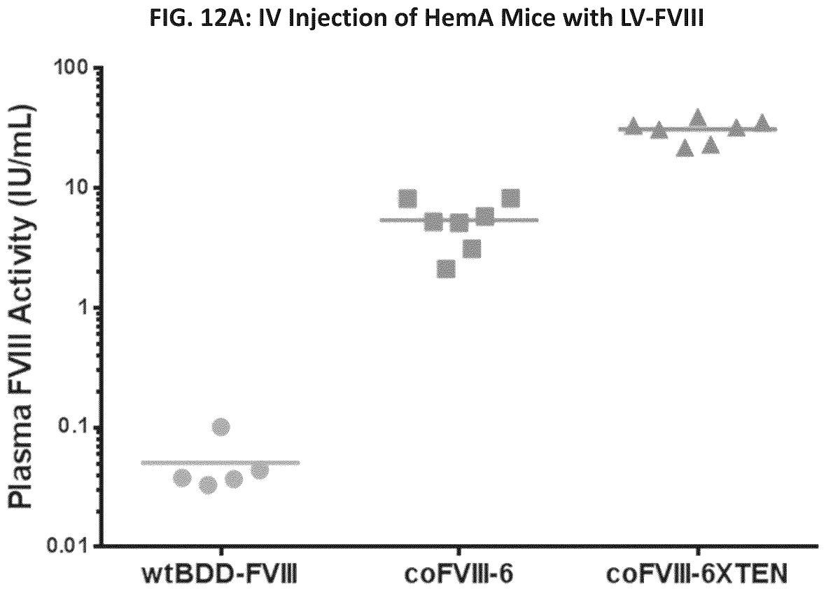

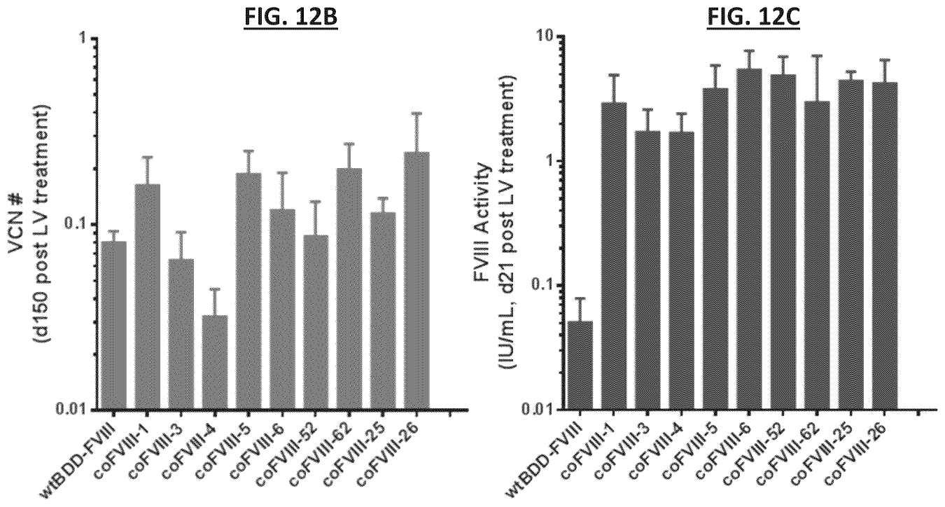

FVIII lentiviral vectors were administered to 14-day-old HemA mouse pups by intravenous injection at a dose of 1.5E10 TU/kg LV-FVIII variant. FVIII plasma activity was measured at day 21 post LV-FVIII treatment, and vector copy number (VCN) per cell was measured in liver necropsy samples collected from LV-FVIII treated animals at day 150 post LV-FVIII treatment. While VCN values were similar in all animals regardless of the LV-FVIII variants administered (FIG. 12B), FVIII activity levels in animals treated with coFVIII variants were 30 to 100-fold higher than in animals treated with wtBDD-FVIII (FIGS. 12A and 12C; Table 7). These data indicate that FVIII codon optimization improves FVIII expression in a lentiviral vector setting.

TABLE 7
Relative expression of codon optimized FVIII constructs

| | LV-FVIII variants | | | | |
|---|---|---|---|---|---|
| | CoFVIII- | CoFVIII- | CoFVIII- | CoFVIII- | CoFVIII- |
| | 1 | 3 | 4 | 5 | 6 |
| Fold- | 57 | 34 | 33 | 74 | 107 |
| improvement | | | | | |
| of FVIII | | | | | |
| expression | | | | | |
| relative to | | | | | |
| LV-wtBDD- | | | | | |
| FVIII | | | | | |

| | LV-FVIII variants | | | |
|---|---|---|---|---|
| | CoFVIII-52 | CoFVIII-62 | CoFVIII-25 | CoFVIII-26 |
| Fold- | 96 | 59 | 87 | 83 |
| improvement | | | | |
| of FVIII | | | | |
| expression | | | | |
| relative to LV- | | | | |
| wtBDD-FVIII | | | | |

### Example 7. FVIII Transgene Expression Mediated Immune Response in HemA Mice Following Lentiviral Treatment

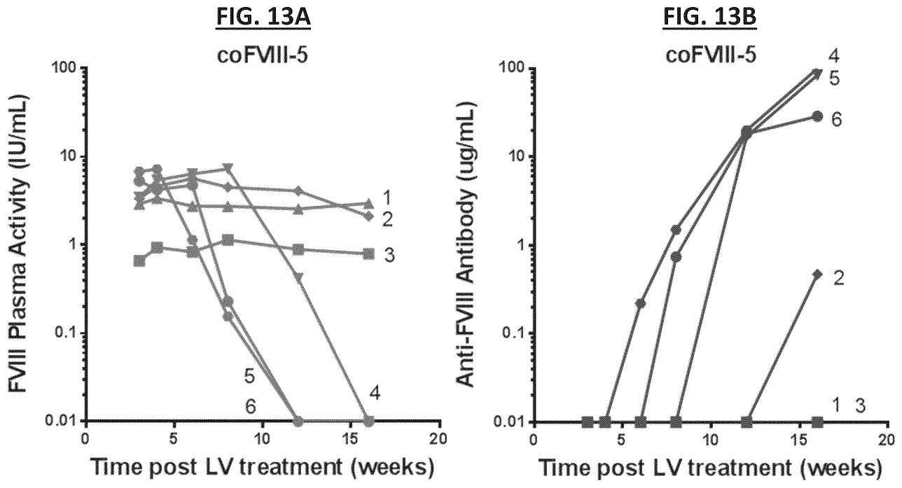

The LV-FVIII-treated mice of Example 6 were evaluated for long-term FVIII expression and anti-FVIII antibody formation. FVIII expression, as evidenced by FVIII plasma activity, varied among animals within the same treatment group (FIG. 13A). For example, three mice (designated 1, 2, and 3) treated with a lentiviral vector expressing the coFVIII-5 variant showed consistent FVIII expression over approximately 16 weeks, whereas three littermates (designated 4, 5, and 6), which were treated with the same lentiviral vector, showed sharp declines in FVIII plasma activity levels by about 10 weeks post treatment (FIG. 13A). The consistent FVIII plasma activity observed in mice 1, 2, and 3 correlated with non-detectable or very low levels of anti-FVIII antibodies (FIG. 13B; mice 1, 2, and 3). Conversely, the mice that exhibited sharp declines in FVIII plasma activity also exhibited increased levels of anti-FVIII antibodies (FIG. 13B; mice 4, 5, and 6). These data suggest that FVIII transgene expression induces anti-FVIII antibody formation in a subset of animals, and the resulting anti-FVIII antibodies eliminated transgenic FVIII protein from circulation.

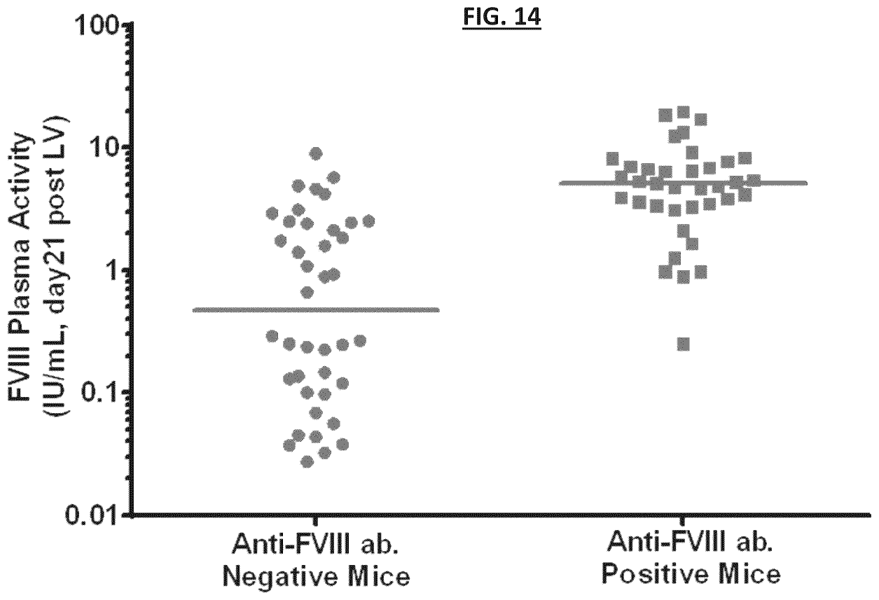

The relationship between FVIII expression and anti-FVIII antibody formation was assessed. The LV-FVIII-treated mice of Example 6 were divided into two groups: mice that were anti-FVIII antibody negative and mice that were anti-FVIII antibody positive. As shown in FIG. 14, expression of transgenic FVIII at physiological levels does not induce an immune response to the transgenic FVIII (FIG. 14, circles) However, supra physiological levels of FVIII expression appears to induce anti-FVIII antibody formation, such that the higher the FVIII expression level, the higher the chance of anti-FVIII antibody induction. These data suggest that it may be beneficial to maintain physiological levels of FVIII expression in patients subjected to a FVIII gene therapy treatment.

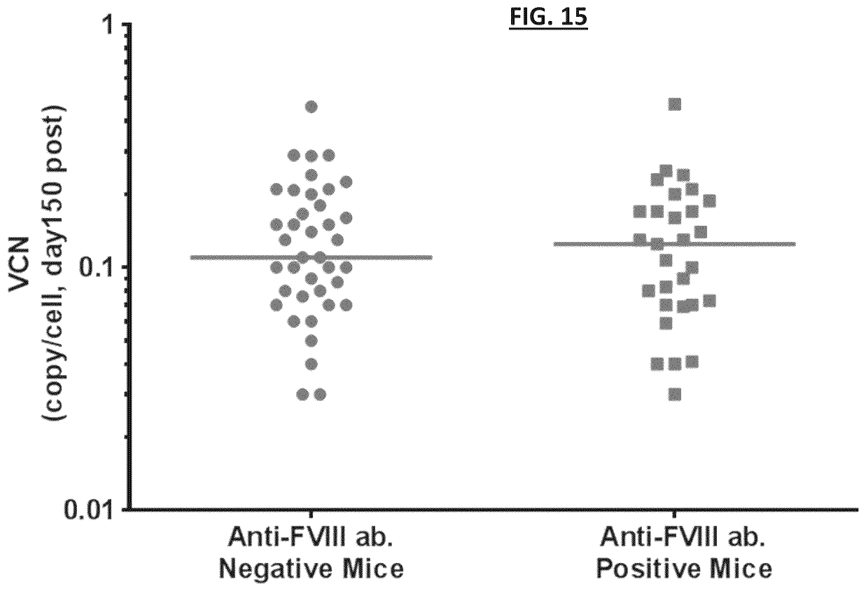

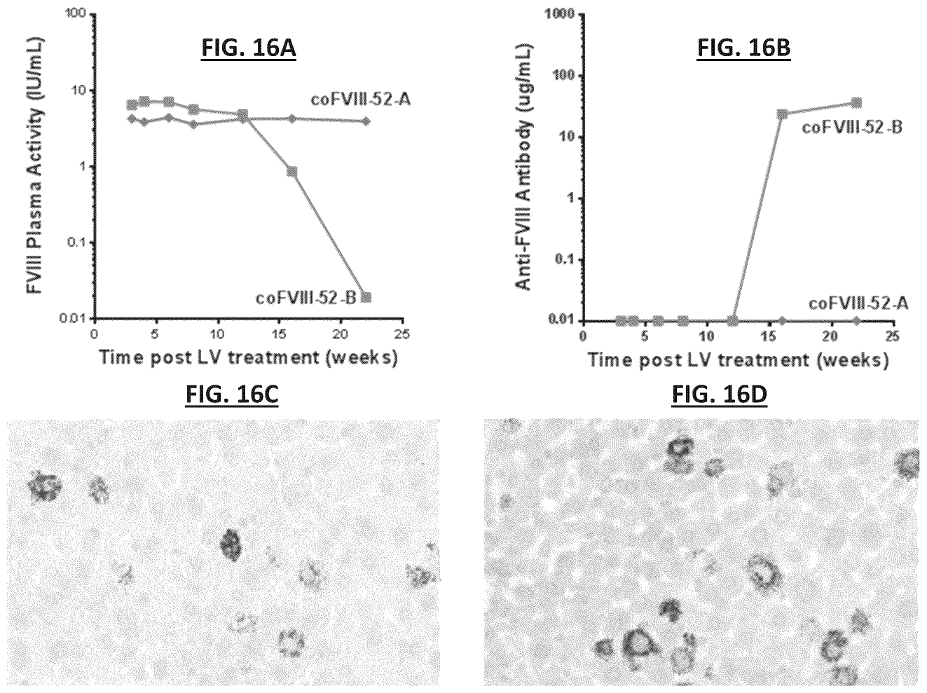

To determine if FVIII expression induced immune response results in loss of transgene expressing liver cells, vector copy number (FIG. 15) and FVIII RNA transcription level (FIG. 16) were evaluated in liver necropsy samples from anti-FVIII antibody positive and negative mice. As shown in FIG. 15, the distribution of vector copy number was the same in anti-FVIII antibody positive and negative mice, indicating that cells with LV-FVIII integration were maintained despite the development of anti-FVIII antibody. This suggests that LV-FVIII mediated FVIII transgene expression dose not induce a Cytotoxic T Lymphocyte (CTL) response against FVIII expressing liver cells. To further confirm these results, FVIII RNA transcription was assessed by RNA in situ hybridization (FIGS. 16C and 16D). At the time of liver harvesting, mouse coFVIII-52-B had no detectable circulating FVIII and a high level of anti-FVIII antibodies (FIGS. 16A and 16B). However, the RNA transcription signal and the number of FVIII-RNA positive cells in liver tissue from the coFVIII-52-B mouse were comparable to the FVIII-52-A mouse, which had about 4 IU/ml of circulating FVIII at time of necropsy. Therefor FVIII expression did not induce CTL response in experimental HemA mice.

### Example 8. FVIII Long-Term Expression in LV-FVIII Treated HemA Mouse Neonates

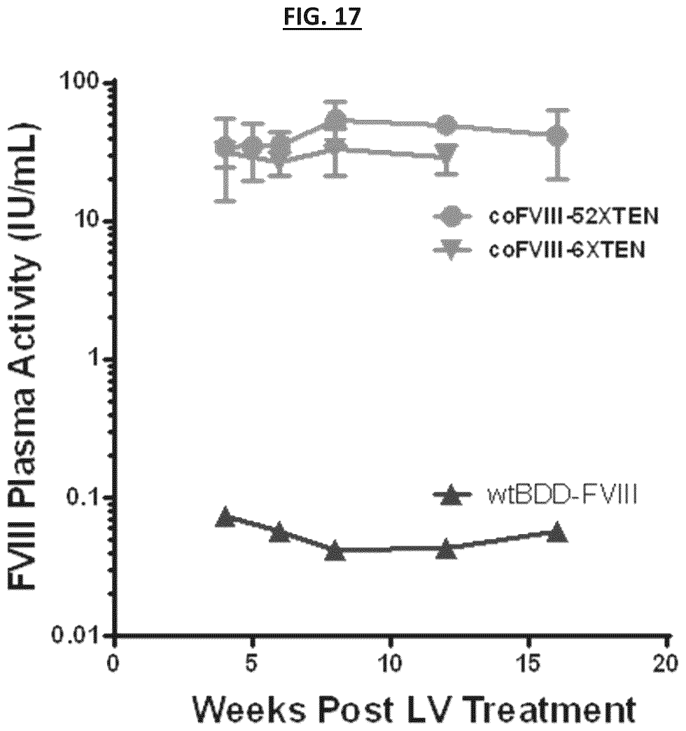

To assess the efficacy of using a lentiviral system for the treatment of pediatric HemA patients through targeting the liver, 2-day-old HemA mice were administered by temple vein injection about 1.5 E10 TU/kg of LV-coFVIII-52XTEN, LV-coFVIII6-XTEN, or a lentiviral vector expressing wtBDD-FVIII. Consistent long-term FVIII expression was observed for both variants and the control, demonstrating that the integrated FVIII expression cascade was maintained in the dividing liver cells of the treated mice (FIG. 17). These data suggest that LV-FVIII could potentially be used to treat both pediatric and adult HemA patients.

### Example 9. Evaluation of LV-FVIII in HemA Dog Neonates

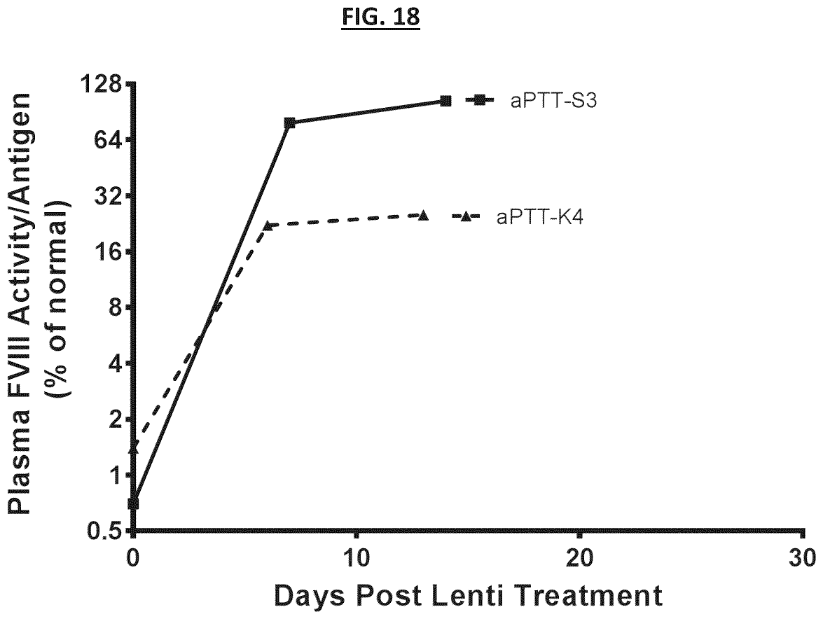

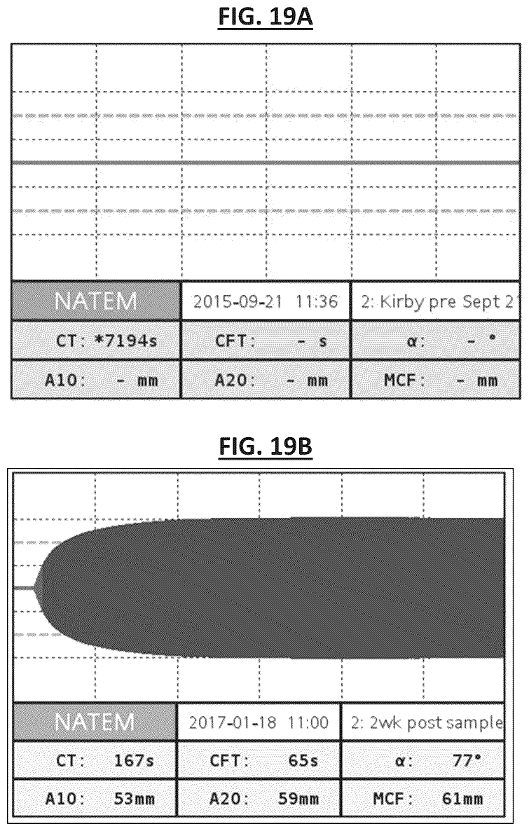

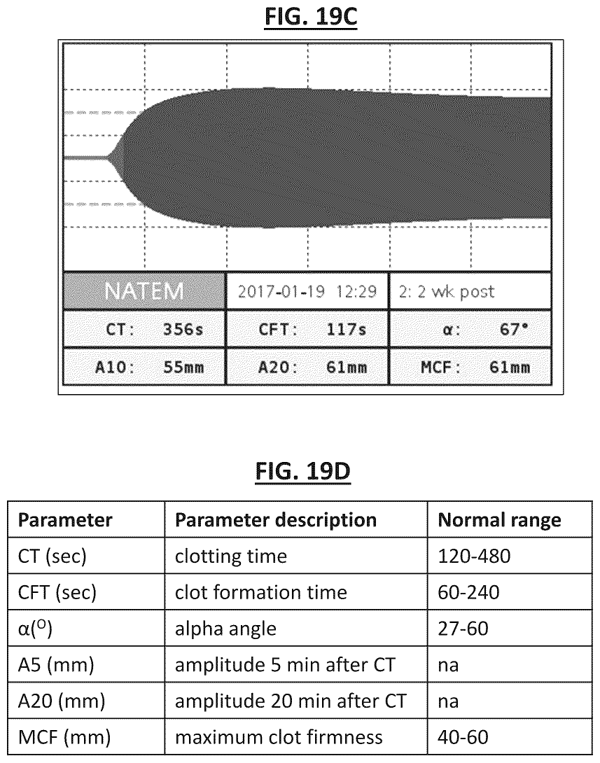

To further evaluate the efficacy of LV-FVIII in larger animal models, two one-week old HemA dog neonates (designated S3 and K4) were administered by intravenous injection 1.3×10⁹ TU/kg LV-coFVIII-6-XTEN. This dose was more than 10-fold lower than the dose previously used in HemA mouse models. Following administration of the lentiviral vectors, plasma FVIII activity was monitored by one stage clotting assay (aPTT) (FIG. 18) and whole blood hemostasis was monitored by rotational thromboelastometry (ROTEM) assay (FIGS. 19A-19D). Before LV-FVIII treatment, the FVIII level for S3 was 0.7% of normal (FIG. 18). Post lentiviral vector treatment, the FVIII level of S3 increased to 79% and 103% of normal at day 7 and day 14, respectively (FIG. 18). For K4, the pre-administration FVIII level was 1.4% of normal (FIG. 18). After lentiviral vector treatment, the FVIII level increased to 22% and 25% of normal at day 6 and day 14, respectively (FIG. 18).

Correlated to the FVIII level, normalized ROTEM was observed for both animals at 2 weeks post treatment (FIGS. 19A-19C), demonstrating the therapeutic benefit mediated by LV-FVIII. The therapeutically beneficial FVIII expression level achieved by LV-FVIII in HemA dog, confirms the potential use of LV-FVIII for the treatment of Hemophilia A.

### Example 10. Evaluation of LV-FVIII in HemA Mouse Neonates

Ex vivo gene therapy with lentiviral vectors (LV) for gene replacement has demonstrated clinical efficacy for multiple indications and with multi-year follow up in treated patients showing no evidence of tumorigenesis. Systemic delivery of LV-FIX mediates persistent FIX expression and is well tolerated in hemophilia animal models. The large packaging capacity, ability to sustain long-term transgene expression via gene integration, lack of pre-existing anti-LV antibodies (abs) in human populations and the encouraging in vivo profiles demonstrated in pre-clinical and clinical settings, make LV a promising vehicle for in vivo gene delivery, especially for gene candidates with large cDNA size such as FVIII.

To evaluate the potential use of LV-FVIII for the treatment of hemophilia A (HemA), codon optimized Human FVIII (hFVIII) variants placed under a hepatocyte-specific promoter were built into a LV system that contains multiple copies of microRNA-142 target sequences to minimize FVIII expression in antigen presenting cells and reduce the probability of inducing anti-FVIII antibodies. LV-hFVIII vectors were produced by transient-transfection of 293T cells, followed by 1000-fold concentration by ultra-centrifugation and evaluated in HemA mouse models. Post intravenous administration of LV-hFVIII, circulating hFVIII level was monitored by FVIII activity and antigen assays, LV transduction efficiency in the liver was assessed by measuring LV DNA copies via quantitative PCR and transgene RNA via In Situ Hybridization, anti-hFVIII antibodies were measured by total anti-hFVIII antibody ELISA.

Persistent FVIII expression was observed for all LV-hFVIII variants in HemA mice that were treated at the neonatal stage. At 1.5E10 transducing units/kg dose, LV encoding codon optimized hFVIII (LV-cohFVlll) resulted in 30 to 100-fold higher circulating FVIII than LV encoding wild type hFVIII (FIG. 12C), while the vector copy number in liver cells and percent of FVIII RNA positive cells were comparable in all tested groups (FIG. 12B). Combination of codon optimization with XTEN (LV-cohFVlll-XTEN), a non-structured hydrophilic poly-peptide that presumably improves the circulating half-life by increasing the hydrodynamic size of the payload, resulted in 30-50 IU/mL FVIII activity in plasma, representing 3,000 to 5,000% of normal circulating FVIII level (FIG. 12A, FIG. 17). Furthermore, anti-hFVIII abs were only detected in mice with supra physiological level of hFVIII (FIG. 14), but no cytotoxic T lymphocyte response against LV transduced cells was observed in anti-hFVIII antibody positive mice (FIGS. 15 and 16A-16D). Our result supports further development of LV-FVIII for in vivo gene therapy of hemophilia A.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1-15. (canceled)

16. An isolated nucleic acid molecule comprising a nucleotide sequence encoding a polypeptide with FVIII activity, wherein the nucleotide sequence comprises:
(i) at least 91%, at least 95%, at least 96%, at least 97%, at least 98%, or at least 99% sequence identity to nucleotides 58-2277 and 2320-4374 of SEQ ID NO: 1;
(ii) at least 94%, at least 95%, at least 96%, at least 97%, at least 98%, or at least 99% sequence identity to nucleotides 58-2277 and 2320-4374 of SEQ ID NO: 2;
(iii) at least 90%, at least 91%, at least 95%, at least 96%, at least 97%, at least 98%, or at least 99% sequence identity to nucleotides 58-2277 and 2320-4374 of SEQ ID NO: 70;
(iv) at least about 92%, at least about 93%, at least about 94%, at least about 95%, at least 96%, at least 97%, at least 98% or at least 99% sequence identity to nucleotides 58-2277 and 2320-4374 of SEQ ID NO: 3;
(v) at least about 91%, at least about 92%, at least about 93%, at least about 94%, at least about 95%, at least 96%, at least 97%, at least 98% or at least 99% sequence identity to nucleotides 58-2277 and 2320-4374 of SEQ ID NO: 4;
(vi) at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 5; or
(vii) at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 6.

17. The isolated nucleic acid molecule of claim 16, wherein the nucleotide sequence comprises at least 92% sequence identity to nucleotides 58-2277 and 2320-4374 of SEQ ID NO: 3.

18. The isolated nucleic acid molecule of claim 16, wherein the nucleotide sequence further comprises a nucleic acid sequence encoding a signal peptide, wherein the nucleic acid sequence encoding a signal peptide has at least 60%, at least 70%, at least 80%, at least 90%, at least 95%, at least 96%, at least 97%, at least 98%, at least 99%, or 100% sequence identity to:
(i) nucleotides 1 to 57 of SEQ ID NO: 1;
(ii) nucleotides 1 to 57 of SEQ ID NO: 2;
(iii) nucleotides 1 to 57 of SEQ ID NO: 3;
(iv) nucleotides 1 to 57 of SEQ ID NO: 4;
(v) nucleotides 1 to 57 of SEQ ID NO: 5;
(vi) nucleotides 1 to 57 of SEQ ID NO: 6;
(vii) nucleotides 1 to 57 of SEQ ID NO: 70;
(viii) nucleotides 1 to 57 of SEQ ID NO: 71; or
(ix) nucleotides 1 to 57 of SEQ ID NO: 68.

19. The isolated nucleic acid molecule of claim 16, wherein the nucleic acid molecule comprises one or more property selected from the group consisting of:
(a) the human codon adaptation index the nucleic acid molecule or a portion thereof is increased relative to SEQ ID NO: 16;
(b) the frequency of optimal codons of the nucleotide sequence or a portion thereof is increased relative to SEQ ID NO:16;
(c) the nucleotide sequence or a portion thereof contains a higher percentage of G/C nucleotides compared to the percentage of G/C nucleotides in SEQ ID NO: 16;
(d) the relative synonymous codon usage of the nucleotide sequence or a portion thereof is increased relative to SEQ ID NO: 16;
(e) the effective number of codons of the nucleotide sequence or a portion thereof is reduced relative SEQ ID NO: 16;
(f) the nucleotide sequence contains fewer MARS/ARS sequences (SEQ ID NOs: 21 and 22) relative to SEQ ID NO: 16;
(g) the nucleotide sequence contains fewer destabilizing elements (SEQ ID NOs: 23 and 24) relative to SEQ ID NO: 16; and
(h) any combination thereof

20. The isolated nucleic acid molecule of claim 16, further comprising a heterologous nucleotide sequence encoding a heterologous amino acid sequence, optionally wherein the heterologous amino acid sequence is an immunoglobulin constant region or a portion thereof, XTEN, transferrin, albumin, or a PAS sequence.

21. The isolated nucleic acid molecule of claim 20, wherein the isolated nucleic acid molecule comprises a heterologous nucleotide sequence encoding an XTEN and the nucleotide sequence of the isolated nucleic acid molecule comprises SEQ ID NO: 19.

22. The isolated nucleic acid molecule of claim 20, wherein the heterologous amino acid sequence is linked to the N-terminus or the C-terminus of the amino acid sequence encoded by the nucleotide sequence or inserted between two amino acids in the amino acid sequence encoded by the nucleotide sequence at one or more insertion site selected from Table 3.

23. The isolated nucleic acid molecule of claim 16, wherein the FVIII polypeptide is a full length FVIII or a B domain deleted FVIII.

24. A vector comprising the nucleic acid molecule of claim 16, optionally wherein the vector is a lentiviral vector.

25. A host cell comprising the nucleic acid molecule of claim 16.

26. A host cell comprising the vector of claim 24.

27. A method of producing a polypeptide with FVIII activity, comprising:
culturing the host cell of claim 25 under conditions whereby a polypeptide with FVIII activity is produced, and
recovering the polypeptide with FVIII activity.

28. An isolated nucleic acid molecule comprising a nucleotide sequence encoding a polypeptide with FVIII activity, wherein the nucleotide sequence comprises:
(i) at least 90% sequence identity to SEQ ID NO: 71;
(ii) at least 90% sequence identity to SEQ ID NO: 72;
(ii) at least 90% sequence identity to SEQ ID NO: 1;
(iii) at least 94% sequence identity to SEQ ID NO: 2;
(iv) at least 90% sequence identity to SEQ ID NO: 70;
(v) at least 90% sequence identity to SEQ ID NO: 3;
(vi) at least 91% sequence identity to SEQ ID NO: 4;
(vii) at least 90% sequence identity to SEQ ID NO: 5; or
(viii) at least 90% sequence identity to SEQ ID NO: 6.

29. A method of treating a bleeding disorder comprising:
administering to a subject in need a FVIII polypeptide encoded by any one of the nucleic acid molecules of claim 28.

30. The method of claim 29, wherein the bleeding disorder is hemophilia.

31. The method of claim 29, wherein administering the isolated nucleic acid molecule comprising SEQ ID NO: 71 or SEQ ID NO: 72 to a subject in need thereof increases expression of a polypeptide with FVIII activity in a subject wherein the expression of the polypeptide is increased relative to a reference nucleic acid molecule comprising SEQ ID NO: 16.

32. The polypeptide encoded by the isolated nucleic acid molecule of claim 28.


SEQUENCE LISTING (103 sequences)

SEQ ID NO: 1 - DNA, 4374 bp, synthetic construct
  misc_feature 1..4374: note = coFVIII-5
  source 1..4374: mol_type = other DNA
atgcaaatcgaactgagcacctgtttcttcctctgcctgctgagattctgtttctccgcgacccgccgatactacctgggagcagtggagctctcctgggattacatgcagagcgaccttggggagctgcccgtggatgccaggttccctccccgggtgccaaagtcgtttccgttcaacacctccgtggtgtacaagaaaactctgttcgtggagttcaccgaccacctgttcaatatcgccaagcccagacctccctggatggggctgttgggacctaccatccaagcggaggtgtacgacactgtggtcatcactctgaagaacatggcctcgcatcccgtgtccctgcacgccgtgggagtgtcttactggaaagcgtccgagggggccgaatacgacgaccagacctcgcagagagaaaaggaagatgacaaggtgttcccaggaggatcgcacacctacgtgtggcaagtgttgaaggagaacggcccaatggcctccgacccgctgtgcctgacctactcgtacctgtcccacgtggacctcgtgaaggacctcaactcgggactgattggagccctgctggtctgcagggaaggctcactggcgaaagaaaagactcagaccttgcacaagttcattctgctgttcgctgtgttcgacgaggggaagtcgtggcacagcgagactaagaactccctgatgcaagatagagatgccgcctccgcccgggcctggcctaagatgcacaccgtgaacggttacgtgaaccgctccctccctggcctgattggatgccaccggaagtccgtgtactggcacgtgatcgggatggggaccacccccgaggtgcacagcatcttcctggaaggtcacacatttctcgtgcgcaaccaccggcaggcctccctggaaatcagccccattaccttcctcactgcccagactctgctgatggacctgggacagttcctgctgttctgccatatctcctcccaccaacatgacggaatggaggcatacgtgaaggtcgattcctgccctgaggaaccccagctccgcatgaagaacaatgaggaagccgaggactacgacgacgacctgacggatagcgagatggatgtggtccggttcgatgacgataacagcccttccttcatccaaattcgctcggtggcaaagaagcaccccaagacctgggtgcattacattgcggcggaagaagaggactgggattatgccccgcttgtcctcgctcctgacgaccggagctacaagagccagtacctgaacaacggtccacagaggatcggtagaaagtacaagaaggtccgcttcatggcctataccgacgaaaccttcaaaactagagaggccatccaacacgaatccggcatcctgggcccgctcttgtacggagaagtcggcgacacccttctcattatcttcaagaaccaggcttcccggccgtacaacatctatccgcatgggatcactgacgtgcgcccactgtactcgcggcgcctgcccaagggtgtcaaacacctgaaggattttccgatccttccgggagaaatcttcaagtacaagtggaccgtgaccgtggaagatggcccaactaagtctgaccctagatgcctcacccgctactactcatccttcgtcaacatggagcgcgacctggccagcggactgatcggcccgctgctgatttgctacaaggaatcagtggaccaacggggaaaccagatcatgtcggataagaggaacgtcatcctcttctccgtgtttgacgaaaaccggtcgtggtacctgactgaaaacatccagcggttcctccccaaccccgcgggcgtgcagctggaagatcctgagtttcaggcatcaaacatcatgcactccattaacggctacgtgttcgattcgctgcagctgagcgtgtgtctgcacgaagtggcctactggtacatcctgtccattggtgcccagactgacttcctgtccgtgtttttctccggctacacgttcaagcacaagatggtgtacgaggacaccctgaccctcttccctttttccggcgaaactgtgtttatgagcatggagaatcccggcctgtggatcttgggctgccacaacagcgacttccgtaacagaggaatgactgcgctgctcaaggtgtccagctgcgacaagaacaccggagactattatgaggactcatacgaggacatctccgcctacctcctgtccaagaataacgccattgaacctcggagcttcagccagaacccacccgtgcttaagagacatcaacgggagatcactaggaccaccctgcagtcagaccaggaggaaatcgactacgatgacaccatctcggtcgagatgaagaaggaggactttgacatctacgacgaagatgaaaaccagagcccgaggtcgttccaaaagaaaacccgccactactttattgctgctgtcgagcggctgtgggactacggaatgtcgtcctcgccgcacgtgctccgcaaccgagcccagagcggctcggtgccgcaattcaagaaggtcgtgttccaggagttcactgacgggagcttcactcagcctttgtaccggggagaactcaatgaacatctcggcctcctcggaccttacatcagagcagaagtggaagataacatcatggtcactttccgtaaccaagccagccgcccgtactcgttctactcctccctcatttcttacgaagaggaccagcggcagggcgcagaaccgcgcaagaacttcgtgaagcccaacgaaaccaagacctacttctggaaagtgcagcatcatatggccccgactaaggacgagtttgactgcaaagcctgggcctacttctccgatgtggacttggagaaggacgtccactccggcctcatcggtcccctgctcgtgtgccataccaataccctgaaccccgcacacggtcgccaggtcaccgtgcaggagttcgctctgttcttcactatcttcgacgaaactaagtcctggtacttcaccgagaacatggagaggaactgcagagccccctgtaacatccagatggaggacccgacgttcaaggaaaactaccggttccacgccattaacggatacatcatggatacgctgccgggtcttgtgatggcccaggatcaacggatcagatggtacttattgtcgatgggcagcaacgagaacatccactctattcacttctccggtcatgtgttcactgtgcggaagaaggaagagtacaagatggccctgtacaacctttatcccggagtgttcgaaactgtggaaatgctgccgtcgaaggccggcatttggcgcgtggagtgtttgattggagaacatctccatgcggggatgtcaaccctgttcctggtgtatagcaacaagtgccagactccgcttgggatggcgtcaggacacattagggatttccagatcactgcgtccggccagtacggccaatgggcccctaagctggcccgcctgcattactccggatccattaacgcctggtcaaccaaggagccattctcctggatcaaggtggaccttctggcccccatgattatccacggaattaagacccagggggcccggcagaagttctcctcactgtacatcagccagttcataatcatgtactccctggacggaaagaagtggcaaacctacagggggaacagcaccggcacactgatggtctttttcggaaatgtggactcctccgggattaagcataacatcttcaaccctccgattatcgctcggtacattagacttcaccctacccactacagcattcgctccaccctgcggatggaactgatgggctgcgatctgaactcgtgcagcatgccgttgggaatggagtccaaagcaatttccgacgcgcagatcaccgcctcgtcctactttaccaacatgttcgccacgtggtcaccgtccaaggcccggctgcacctccagggaagatccaacgcatggcggccacaggtcaacaaccctaaggagtggctccaggtggacttccagaaaaccatgaaggtcaccggagtcacaacccagggagtgaagtcgctgctgacttctatgtacgtcaaggagttcctgatctccagcagccaggacgggcaccagtggaccctgttcttccaaaatggaaaggtcaaggtgtttcagggcaatcaggattcattcaccccggtggtgaactcccttgatccacccctcctgacccgctaccttcgcatccacccacagtcctgggtgcaccagatcgcgctgaggatggaggtcctgggatgcgaagcccaggacctgtactga

SEQ ID NO: 2 - DNA, 4374 bp, synthetic construct
  misc_feature 1..4374: note = coFVIII-4
  source 1..4374: mol_type = other DNA
atgcagatcgagctgagcacgtgcttcttcctgtgcctgctgaggttctgcttcagcgccaccaggaggtactacctgggcgccgtggagctgagctgggactacatgcagagcgacctgggcgagctgcccgtggacgccaggttcccccccagggtgcccaagagcttccccttcaacacgagcgtggtgtacaagaagaccctgttcgtggagttcaccgaccatctgttcaatatcgccaagcccaggcccccctggatggggctgctggggcccacgatccaggccgaggtgtacgacaccgtggtcatcaccctgaagaacatggccagccaccccgtgagcctgcacgccgtgggcgtgagctactggaaggccagcgagggcgccgagtacgacgaccagaccagccagagggagaaggaggacgacaaggtgttccccggcggcagccacacctacgtgtggcaggtgctgaaggagaatgggcccatggccagcgaccccctgtgcctgacctactcttacctgagccacgtggatctggtgaaggacctgaacagcggcctgatcggcgccctgctggtgtgcagggagggcagcctggccaaggagaagacccagaccctgcacaagttcatcctgctgttcgccgtgttcgacgagggcaagagctggcacagcgagaccaagaacagcctgatgcaggatagggacgccgccagcgccagggcctggcccaagatgcacaccgtgaacggctacgtgaacaggtctctgcccggcctgatcggctgccacaggaagagcgtgtactggcacgtgatcggcatggggaccacccccgaggtgcacagcatcttcctggagggccacacgttcctggtgaggaatcacaggcaggccagcctggagatcagcccgatcaccttcctgaccgcccagaccctgctgatggacctggggcagttcctgctgttctgccatatcagctctcaccagcacgacggcatggaggcctacgtgaaggtggatagctgccccgaggagccccagctgaggatgaagaacaacgaggaggccgaggactacgacgacgacctgaccgacagcgagatggacgtggtgaggttcgacgacgacaatagcccgagcttcatccagatcaggagcgtggccaagaagcaccccaagacctgggtgcattacatcgccgccgaggaggaggattgggactacgcccccctggtgctggcccccgacgacaggtcttacaagagccagtacctgaacaacgggccccagaggatcggcaggaagtacaagaaggtgaggttcatggcctacaccgacgagaccttcaagaccagggaggcgatccagcacgagagcgggatcctggggcccctgctgtacggcgaggtgggcgacacgctgctgatcatcttcaagaaccaggccagcaggccgtacaatatctacccccacgggatcaccgacgtgaggcccctgtactctaggaggctgcccaagggcgtgaagcacctgaaggacttccccatcctgcccggcgagatcttcaagtacaagtggaccgtgaccgtggaggacgggcccacgaagagcgaccccaggtgcctgaccaggtactacagctctttcgtgaacatggagagggacctggccagcggcctgatcgggcccctgctgatctgctacaaggagagcgtggatcagaggggcaaccagatcatgagcgacaagaggaacgtgatcctgttcagcgtgttcgacgagaataggtcttggtacctgaccgagaatatccagaggttcctgcccaaccccgccggcgtgcagctggaggatcccgagttccaggccagcaacatcatgcacagcatcaacggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttcagcggcgagaccgtgttcatgagcatggagaaccccggcctgtggatcctgggctgccataactccgacttcaggaataggggcatgaccgccctgctgaaggtgagctcttgcgacaagaacaccggcgactactacgaggatagctacgaggatatcagcgcctacctgctgagcaagaacaacgccatcgagcccaggtctttcagccagaacccccccgtgctgaagaggcaccagagggagatcaccaggacgaccctgcagagcgaccaggaggagatcgactacgacgacacgatcagcgtggagatgaagaaggaggatttcgacatctacgacgaggacgagaatcagagccccaggtctttccagaagaagaccaggcattacttcatcgccgccgtggagaggctgtgggactacggcatgagcagctctccccacgtgctgaggaatagggcccagagcggcagcgtgccccagttcaagaaggtggtgttccaggagttcaccgacggcagcttcacccagcccctgtacaggggcgagctgaacgagcacctgggcctgctggggccctacatcagggccgaggtggaggataacatcatggtgaccttcaggaatcaggccagcaggccctatagcttctatagctctctgatcagctacgaggaggatcagaggcagggcgccgagcccaggaagaacttcgtgaagcccaacgagaccaagacctacttctggaaggtgcagcaccacatggcccccacgaaggacgagttcgactgcaaggcctgggcctacttcagcgacgtggatctggagaaggacgtgcacagcggcctgatcgggcccctgctggtgtgccacaccaacaccctgaaccccgcccacggcaggcaggtgaccgtgcaggagttcgccctgttcttcaccatcttcgacgagaccaagagctggtacttcaccgagaatatggagaggaattgcagggccccctgcaatatccagatggaggacccgaccttcaaggagaattacaggttccacgccatcaacggctacatcatggacacgctgcccggcctggtcatggcccaggatcagaggatcaggtggtatctgctgagcatggggagcaacgagaatatccacagcatccacttcagcggccacgtgttcaccgtgaggaagaaggaggagtacaagatggccctgtacaatctgtaccccggcgtgttcgagaccgtggagatgctgcccagcaaggccgggatctggagggtggagtgcctgatcggcgagcacctgcacgccggcatgagcacgctgttcctggtgtactctaacaagtgccagacccccctggggatggccagcggccacatcagggacttccagatcaccgccagcggccagtacggccagtgggcccccaagctggccaggctgcactattccggaagcatcaacgcctggagcacgaaggagcccttcagctggatcaaggtggatctgctggcccccatgatcatccacgggatcaagacccagggcgccaggcagaagttcagctctctgtatatcagccagttcatcatcatgtactctctggacggcaagaagtggcagacctacaggggcaacagcaccggcacgctgatggtgttcttcggcaacgtggactctagcgggatcaagcacaatatcttcaacccccccatcatcgccaggtacatcaggctgcaccccacccattactctatcaggtctaccctgaggatggagctgatgggctgcgacctgaacagctgcagcatgcccctggggatggagagcaaggccatcagcgacgcccagatcaccgccagctcttacttcaccaacatgttcgccacctggagcccgagcaaggccaggctgcacctgcagggcaggtctaacgcctggaggccccaggtgaacaaccccaaggagtggctgcaggtggatttccagaagaccatgaaggtgaccggcgtgaccacgcagggcgtgaagagcctgctgaccagcatgtacgtgaaggagttcctgatcagctctagccaggacggccaccagtggaccctgttcttccagaacggcaaggtgaaggtgttccagggcaaccaggatagcttcacccccgtggtgaacagcctggacccccccctgctgaccaggtatctgaggatccacccccagagctgggtgcaccagatcgccctgaggatggaggtgctgggctgcgaggcccaggatctgtattga

SEQ ID NO: 3 - DNA, 4374 bp, synthetic construct
  misc_feature 1..4374: note = coFVIII-52
  source 1..4374: mol_type = other DNA
atgcaaatcgaactgagcacctgtttcttcctctgcctgctgagattctgtttctccgcgacccgccgatactacctgggagcagtggagctctcctgggattacatgcagagcgaccttggggagctgcccgtggatgccaggttccctccccgggtgccaaagtcgtttccgttcaacacctccgtggtgtacaagaaaactctgttcgtggagttcaccgaccacctgttcaatatcgccaagcccagacctccctggatggggctgttgggacctaccatccaagcggaggtgtacgacactgtggtcatcactctgaagaacatggcctcgcatcccgtgtccctgcacgccgtgggagtgtcttactggaaagcgtccgagggggccgaatacgacgaccagacctcgcagagagaaaaggaagatgacaaggtgttcccaggaggatcgcacacctacgtgtggcaagtgttgaaggagaacggcccaatggcctccgacccgctgtgcctgacctactcgtacctgtcccacgtggacctcgtgaaggacctcaactcgggactgattggagccctgctggtctgcagggaaggctcactggcgaaagaaaagactcagaccttgcacaagttcattctgctgttcgctgtgttcgacgaggggaagtcgtggcacagcgagactaagaactccctgatgcaagatagagatgccgcctccgcccgggcctggcctaagatgcacaccgtgaacggttacgtgaaccgctccctccctggcctgattggatgccaccggaagtccgtgtactggcacgtgatcgggatggggaccacccccgaggtgcacagcatcttcctggaaggtcacacatttctcgtgcgcaaccaccggcaggcctccctggaaatcagccccattaccttcctcactgcccagactctgctgatggacctgggacagttcctgctgttctgccatatctcctcccaccaacatgacggaatggaggcatacgtgaaggtcgattcctgccctgaggaaccccagctccgcatgaagaacaatgaggaagccgaggactacgacgacgacctgacggatagcgagatggatgtggtccggttcgatgacgataacagcccttccttcatccaaattcgctcggtggcaaagaagcaccccaagacctgggtgcattacattgcggcggaagaagaggactgggattatgccccgcttgtcctcgctcctgacgaccggagctacaagagccagtacctgaacaacggtccacagaggatcggtagaaagtacaagaaggtccgcttcatggcctataccgacgaaaccttcaaaactagagaggccatccaacacgaatccggcatcctgggcccgctcttgtacggagaagtcggcgacacccttctcattatcttcaagaaccaggcttcccggccgtacaacatctatccgcatgggatcactgacgtgcgcccactgtactcgcggcgcctgcccaagggtgtcaaacacctgaaggattttccgatccttccgggagaaatcttcaagtacaagtggaccgtgaccgtggaagatggcccaactaagtctgaccctagatgcctcacccgctactactcatccttcgtcaacatggagcgcgacctggccagcggactgatcggcccgctgctgatttgctacaaggaatcagtggaccaacggggaaaccagatcatgtcggataagaggaacgtcatcctcttctccgtgtttgacgaaaaccggtcgtggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaaccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 4 - DNA, 4374 bp, synthetic construct
  misc_feature 1..4374: note = coFVIII-62
  source 1..4374: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaaccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 5 - DNA, 4374 bp, synthetic construct
  misc_feature 1..4374: note = coFVIII-25
  source 1..4374: mol_type = other DNA
atgcagattgagctgagcacctgcttcttcctgtgcctgctgaggttctgcttctctgccaccaggagatactacctgggcgccgtggagctgagctgggactacatgcagtctgacctgggcgagctgccagtggacgccaggttcccccccagagtgcccaagagcttccccttcaacaccagcgtggtgtacaagaagaccctgttcgtggagttcactgaccacctgttcaacatcgccaagcccaggcccccctggatgggcctgctgggccccaccatccaggccgaggtgtacgacaccgtggtcatcaccctgaagaacatggccagccaccccgtctccctgcacgccgtgggggtgagctactggaaggcctctgagggcgccgagtacgacgaccagaccagccagagggagaaggaggacgacaaggtgttccctgggggcagccacacctacgtgtggcaggtcctgaaggagaacggccccatggcctctgaccccctgtgcctgacctacagctacctgagccacgtggacctggtgaaggacctgaactctggcctgattggggccctgctggtgtgcagggagggcagcctggccaaggagaagacccagaccctgcacaagttcatcctgctgttcgccgtgttcgacgagggcaagagctggcactctgaaaccaagaacagcctgatgcaggacagggacgccgcctctgccagggcctggcccaagatgcacaccgtcaacggctacgtcaacaggagcctgcctggcctgattggctgccacaggaagagcgtgtactggcatgtgatcggcatgggcaccacccctgaggtgcacagcatcttcctggagggccacaccttcctggtcaggaaccacaggcaggccagcctggagatcagccccatcaccttcctgaccgcccagaccctgctgatggacctgggccagttcctgctgttctgccacatctccagccaccagcacgacggcatggaggcctacgtgaaagtggacagctgccctgaggagccccagctgaggatgaagaacaacgaggaggccgaggactatgatgacgacctgaccgacagcgagatggacgtggtcaggttcgacgacgacaacagccccagcttcatccagatcaggagcgtggccaagaagcaccccaagacctgggtgcactacatcgctgctgaggaggaggactgggactatgcccccctggtgctggcccctgatgacaggagctacaagagccagtacctgaacaatggcccccagaggattggcaggaagtacaagaaagtcaggttcatggcctacactgatgaaaccttcaagaccagggaggccatccagcatgagtctggcatcctgggccccctgctgtacggggaggtgggggacaccctgctgatcatcttcaagaaccaggccagcaggccctacaacatctacccccatggcatcaccgacgtgaggcccctgtacagcaggaggctgcctaagggggtgaagcacctgaaagacttccccatcctgcctggggagatcttcaagtacaagtggactgtgactgtggaggacggccccaccaagagcgaccccaggtgcctgaccagatactacagcagcttcgtcaacatggagagggacctggcctctggcctgattggccccctgctgatctgctacaaggagtctgtggaccagaggggcaaccagatcatgagcgacaagaggaacgtgatcctgttctctgtcttcgacgagaacaggagctggtacctgactgaaaacatccagcggttcctccccaaccccgcgggcgtgcagctggaagatcctgagtttcaggcatcaaacatcatgcactccattaacggctacgtgttcgattcgctgcagctgagcgtgtgtctgcacgaagtggcctactggtacatcctgtccattggtgcccagactgacttcctgtccgtgtttttctccggctacacgttcaagcacaagatggtgtacgaggacaccctgaccctcttccctttttccggcgaaactgtgtttatgagcatggagaatcccggcctgtggatcttgggctgccacaacagcgacttccgtaacagaggaatgactgcgctgctcaaggtgtccagctgcgacaagaacaccggagactattatgaggactcatacgaggacatctccgcctacctcctgtccaagaataacgccattgaacctcggagcttcagccagaacccacccgtgcttaagagacatcaacgggagatcactaggaccaccctgcagtcagaccaggaggaaatcgactacgatgacaccatctcggtcgagatgaagaaggaggactttgacatctacgacgaagatgaaaaccagagcccgaggtcgttccaaaagaaaacccgccactactttattgctgctgtcgagcggctgtgggactacggaatgtcgtcctcgccgcacgtgctccgcaaccgagcccagagcggctcggtgccgcaattcaagaaggtcgtgttccaggagttcactgacgggagcttcactcagcctttgtaccggggagaactcaatgaacatctcggcctcctcggaccttacatcagagcagaagtggaagataacatcatggtcactttccgtaaccaagccagccgcccgtactcgttctactcctccctcatttcttacgaagaggaccagcggcagggcgcagaaccgcgcaagaacttcgtgaagcccaacgaaaccaagacctacttctggaaagtgcagcatcatatggccccgactaaggacgagtttgactgcaaagcctgggcctacttctccgatgtggacttggagaaggacgtccactccggcctcatcggtcccctgctcgtgtgccataccaataccctgaaccccgcacacggtcgccaggtcaccgtgcaggagttcgctctgttcttcactatcttcgacgaaactaagtcctggtacttcaccgagaacatggagaggaactgcagagccccctgtaacatccagatggaggacccgacgttcaaggaaaactaccggttccacgccattaacggatacatcatggatacgctgccgggtcttgtgatggcccaggatcaacggatcagatggtacttattgtcgatgggcagcaacgagaacatccactctattcacttctccggtcatgtgttcactgtgcggaagaaggaagagtacaagatggccctgtacaacctttatcccggagtgttcgaaactgtggaaatgctgccgtcgaaggccggcatttggcgcgtggagtgtttgattggagaacatctccatgcggggatgtcaaccctgttcctggtgtatagcaacaagtgccagactccgcttgggatggcgtcaggacacattagggatttccagatcactgcgtccggccagtacggccaatgggcccctaagctggcccgcctgcattactccggatccattaacgcctggtcaaccaaggagccattctcctggatcaaggtggaccttctggcccccatgattatccacggaattaagacccagggggcccggcagaagttctcctcactgtacatcagccagttcataatcatgtactccctggacggaaagaagtggcaaacctacagggggaacagcaccggcacactgatggtctttttcggaaatgtggactcctccgggattaagcataacatcttcaaccctccgattatcgctcggtacattagacttcaccctacccactacagcattcgctccaccctgcggatggaactgatgggctgcgatctgaactcgtgcagcatgccgttgggaatggagtccaaagcaatttccgacgcgcagatcaccgcctcgtcctactttaccaacatgttcgccacgtggtcaccgtccaaggcccggctgcacctccagggaagatccaacgcatggcggccacaggtcaacaaccctaaggagtggctccaggtggacttccagaaaaccatgaaggtcaccggagtcacaacccagggagtgaagtcgctgctgacttctatgtacgtcaaggagttcctgatctccagcagccaggacgggcaccagtggaccctgttcttccaaaatggaaaggtcaaggtgtttcagggcaatcaggattcattcaccccggtggtgaactcccttgatccacccctcctgacccgctaccttcgcatccacccacagtcctgggtgcaccagatcgcgctgaggatggaggtcctgggatgcgaagcccaggacctgtactga

SEQ ID NO: 6 - DNA, 4374 bp, synthetic construct
  misc_feature 1..4374: note = coFVIII-26
  source 1..4374: mol_type = other DNA
atgcagattgagctgagcacctgcttcttcctgtgcctgctgaggttctgcttctctgccaccaggagatactacctgggcgccgtggagctgagctgggactacatgcagtctgacctgggcgagctgccagtggacgccaggttcccccccagagtgcccaagagcttccccttcaacaccagcgtggtgtacaagaagaccctgttcgtggagttcactgaccacctgttcaacatcgccaagcccaggcccccctggatgggcctgctgggccccaccatccaggccgaggtgtacgacaccgtggtcatcaccctgaagaacatggccagccaccccgtctccctgcacgccgtgggggtgagctactggaaggcctctgagggcgccgagtacgacgaccagaccagccagagggagaaggaggacgacaaggtgttccctgggggcagccacacctacgtgtggcaggtcctgaaggagaacggccccatggcctctgaccccctgtgcctgacctacagctacctgagccacgtggacctggtgaaggacctgaactctggcctgattggggccctgctggtgtgcagggagggcagcctggccaaggagaagacccagaccctgcacaagttcatcctgctgttcgccgtgttcgacgagggcaagagctggcactctgaaaccaagaacagcctgatgcaggacagggacgccgcctctgccagggcctggcccaagatgcacaccgtcaacggctacgtcaacaggagcctgcctggcctgattggctgccacaggaagagcgtgtactggcatgtgatcggcatgggcaccacccctgaggtgcacagcatcttcctggagggccacaccttcctggtcaggaaccacaggcaggccagcctggagatcagccccatcaccttcctgaccgcccagaccctgctgatggacctgggccagttcctgctgttctgccacatctccagccaccagcacgacggcatggaggcctacgtgaaagtggacagctgccctgaggagccccagctgaggatgaagaacaacgaggaggccgaggactatgatgacgacctgaccgacagcgagatggacgtggtcaggttcgacgacgacaacagccccagcttcatccagatcaggagcgtggccaagaagcaccccaagacctgggtgcactacatcgctgctgaggaggaggactgggactatgcccccctggtgctggcccctgatgacaggagctacaagagccagtacctgaacaatggcccccagaggattggcaggaagtacaagaaagtcaggttcatggcctacactgatgaaaccttcaagaccagggaggccatccagcatgagtctggcatcctgggccccctgctgtacggggaggtgggggacaccctgctgatcatcttcaagaaccaggccagcaggccctacaacatctacccccatggcatcaccgacgtgaggcccctgtacagcaggaggctgcctaagggggtgaagcacctgaaagacttccccatcctgcctggggagatcttcaagtacaagtggactgtgactgtggaggacggccccaccaagagcgaccccaggtgcctgaccagatactacagcagcttcgtcaacatggagagggacctggcctctggcctgattggccccctgctgatctgctacaaggagtctgtggaccagaggggcaaccagatcatgagcgacaagaggaacgtgatcctgttctctgtcttcgacgagaacaggagctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 7 - DNA, 1734 bp, synthetic construct
  misc_feature 1..1734: note = coFVIII-52-NT58
  source 1..1734: mol_type = other DNA
gcgacccgccgatactacctgggagcagtggagctctcctgggattacatgcagagcgaccttggggagctgcccgtggatgccaggttccctccccgggtgccaaagtcgtttccgttcaacacctccgtggtgtacaagaaaactctgttcgtggagttcaccgaccacctgttcaatatcgccaagcccagacctccctggatggggctgttgggacctaccatccaagcggaggtgtacgacactgtggtcatcactctgaagaacatggcctcgcatcccgtgtccctgcacgccgtgggagtgtcttactggaaagcgtccgagggggccgaatacgacgaccagacctcgcagagagaaaaggaagatgacaaggtgttcccaggaggatcgcacacctacgtgtggcaagtgttgaaggagaacggcccaatggcctccgacccgctgtgcctgacctactcgtacctgtcccacgtggacctcgtgaaggacctcaactcgggactgattggagccctgctggtctgcagggaaggctcactggcgaaagaaaagactcagaccttgcacaagttcattctgctgttcgctgtgttcgacgaggggaagtcgtggcacagcgagactaagaactccctgatgcaagatagagatgccgcctccgcccgggcctggcctaagatgcacaccgtgaacggttacgtgaaccgctccctccctggcctgattggatgccaccggaagtccgtgtactggcacgtgatcgggatggggaccacccccgaggtgcacagcatcttcctggaaggtcacacatttctcgtgcgcaaccaccggcaggcctccctggaaatcagccccattaccttcctcactgcccagactctgctgatggacctgggacagttcctgctgttctgccatatctcctcccaccaacatgacggaatggaggcatacgtgaaggtcgattcctgccctgaggaaccccagctccgcatgaagaacaatgaggaagccgaggactacgacgacgacctgacggatagcgagatggatgtggtccggttcgatgacgataacagcccttccttcatccaaattcgctcggtggcaaagaagcaccccaagacctgggtgcattacattgcggcggaagaagaggactgggattatgccccgcttgtcctcgctcctgacgaccggagctacaagagccagtacctgaacaacggtccacagaggatcggtagaaagtacaagaaggtccgcttcatggcctataccgacgaaaccttcaaaactagagaggccatccaacacgaatccggcatcctgggcccgctcttgtacggagaagtcggcgacacccttctcattatcttcaagaaccaggcttcccggccgtacaacatctatccgcatgggatcactgacgtgcgcccactgtactcgcggcgcctgcccaagggtgtcaaacacctgaaggattttccgatccttccgggagaaatcttcaagtacaagtggaccgtgaccgtggaagatggcccaactaagtctgaccctagatgcctcacccgctactactcatccttcgtcaacatggagcgcgacctggccagcggactgatcggcccgctgctgatttgctacaaggaatcagtggaccaacggggaaaccagatcatgtcggataagaggaacgtcatcctcttctccgtg

SEQ ID NO: 8 - DNA, 2583 bp, synthetic construct
  misc_feature 1..2583: note = coFVIII-52-CT
  source 1..2583: mol_type = other DNA
tttgacgaaaaccggtcgtggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaaccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 9 - DNA, 1734 bp, synthetic construct
  misc_feature 1..1734: note = coFVIII-62-NT58
  source 1..1734: mol_type = other DNA
gccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtg

SEQ ID NO: 10 - DNA, 2583 bp, synthetic construct
  misc_feature 1..2583: note = coFVIII-62-CT
  source 1..2583: mol_type = other DNA
tttgatgaaaacagatcctggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaaccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 11 - DNA, 1734 bp, synthetic construct
  misc_feature 1..1734: note = coFVIII-25-NT58
  source 1..1734: mol_type = other DNA
gccaccaggagatactacctgggcgccgtggagctgagctgggactacatgcagtctgacctgggcgagctgccagtggacgccaggttcccccccagagtgcccaagagcttccccttcaacaccagcgtggtgtacaagaagaccctgttcgtggagttcactgaccacctgttcaacatcgccaagcccaggcccccctggatgggcctgctgggccccaccatccaggccgaggtgtacgacaccgtggtcatcaccctgaagaacatggccagccaccccgtctccctgcacgccgtgggggtgagctactggaaggcctctgagggcgccgagtacgacgaccagaccagccagagggagaaggaggacgacaaggtgttccctgggggcagccacacctacgtgtggcaggtcctgaaggagaacggccccatggcctctgaccccctgtgcctgacctacagctacctgagccacgtggacctggtgaaggacctgaactctggcctgattggggccctgctggtgtgcagggagggcagcctggccaaggagaagacccagaccctgcacaagttcatcctgctgttcgccgtgttcgacgagggcaagagctggcactctgaaaccaagaacagcctgatgcaggacagggacgccgcctctgccagggcctggcccaagatgcacaccgtcaacggctacgtcaacaggagcctgcctggcctgattggctgccacaggaagagcgtgtactggcatgtgatcggcatgggcaccacccctgaggtgcacagcatcttcctggagggccacaccttcctggtcaggaaccacaggcaggccagcctggagatcagccccatcaccttcctgaccgcccagaccctgctgatggacctgggccagttcctgctgttctgccacatctccagccaccagcacgacggcatggaggcctacgtgaaagtggacagctgccctgaggagccccagctgaggatgaagaacaacgaggaggccgaggactatgatgacgacctgaccgacagcgagatggacgtggtcaggttcgacgacgacaacagccccagcttcatccagatcaggagcgtggccaagaagcaccccaagacctgggtgcactacatcgctgctgaggaggaggactgggactatgcccccctggtgctggcccctgatgacaggagctacaagagccagtacctgaacaatggcccccagaggattggcaggaagtacaagaaagtcaggttcatggcctacactgatgaaaccttcaagaccagggaggccatccagcatgagtctggcatcctgggccccctgctgtacggggaggtgggggacaccctgctgatcatcttcaagaaccaggccagcaggccctacaacatctacccccatggcatcaccgacgtgaggcccctgtacagcaggaggctgcctaagggggtgaagcacctgaaagacttccccatcctgcctggggagatcttcaagtacaagtggactgtgactgtggaggacggccccaccaagagcgaccccaggtgcctgaccagatactacagcagcttcgtcaacatggagagggacctggcctctggcctgattggccccctgctgatctgctacaaggagtctgtggaccagaggggcaaccagatcatgagcgacaagaggaacgtgatcctgttctctgtc

SEQ ID NO: 12 - DNA, 2583 bp, synthetic construct
  misc_feature 1..2583: note = coFVIII-25-CT
  source 1..2583: mol_type = other DNA
ttcgacgagaacaggagctggtacctgactgaaaacatccagcggttcctccccaaccccgcgggcgtgcagctggaagatcctgagtttcaggcatcaaacatcatgcactccattaacggctacgtgttcgattcgctgcagctgagcgtgtgtctgcacgaagtggcctactggtacatcctgtccattggtgcccagactgacttcctgtccgtgtttttctccggctacacgttcaagcacaagatggtgtacgaggacaccctgaccctcttccctttttccggcgaaactgtgtttatgagcatggagaatcccggcctgtggatcttgggctgccacaacagcgacttccgtaacagaggaatgactgcgctgctcaaggtgtccagctgcgacaagaacaccggagactattatgaggactcatacgaggacatctccgcctacctcctgtccaagaataacgccattgaacctcggagcttcagccagaacccacccgtgcttaagagacatcaacgggagatcactaggaccaccctgcagtcagaccaggaggaaatcgactacgatgacaccatctcggtcgagatgaagaaggaggactttgacatctacgacgaagatgaaaaccagagcccgaggtcgttccaaaagaaaacccgccactactttattgctgctgtcgagcggctgtgggactacggaatgtcgtcctcgccgcacgtgctccgcaaccgagcccagagcggctcggtgccgcaattcaagaaggtcgtgttccaggagttcactgacgggagcttcactcagcctttgtaccggggagaactcaatgaacatctcggcctcctcggaccttacatcagagcagaagtggaagataacatcatggtcactttccgtaaccaagccagccgcccgtactcgttctactcctccctcatttcttacgaagaggaccagcggcagggcgcagaaccgcgcaagaacttcgtgaagcccaacgaaaccaagacctacttctggaaagtgcagcatcatatggccccgactaaggacgagtttgactgcaaagcctgggcctacttctccgatgtggacttggagaaggacgtccactccggcctcatcggtcccctgctcgtgtgccataccaataccctgaaccccgcacacggtcgccaggtcaccgtgcaggagttcgctctgttcttcactatcttcgacgaaactaagtcctggtacttcaccgagaacatggagaggaactgcagagccccctgtaacatccagatggaggacccgacgttcaaggaaaactaccggttccacgccattaacggatacatcatggatacgctgccgggtcttgtgatggcccaggatcaacggatcagatggtacttattgtcgatgggcagcaacgagaacatccactctattcacttctccggtcatgtgttcactgtgcggaagaaggaagagtacaagatggccctgtacaacctttatcccggagtgttcgaaactgtggaaatgctgccgtcgaaggccggcatttggcgcgtggagtgtttgattggagaacatctccatgcggggatgtcaaccctgttcctggtgtatagcaacaagtgccagactccgcttgggatggcgtcaggacacattagggatttccagatcactgcgtccggccagtacggccaatgggcccctaagctggcccgcctgcattactccggatccattaacgcctggtcaaccaaggagccattctcctggatcaaggtggaccttctggcccccatgattatccacggaattaagacccagggggcccggcagaagttctcctcactgtacatcagccagttcataatcatgtactccctggacggaaagaagtggcaaacctacagggggaacagcaccggcacactgatggtctttttcggaaatgtggactcctccgggattaagcataacatcttcaaccctccgattatcgctcggtacattagacttcaccctacccactacagcattcgctccaccctgcggatggaactgatgggctgcgatctgaactcgtgcagcatgccgttgggaatggagtccaaagcaatttccgacgcgcagatcaccgcctcgtcctactttaccaacatgttcgccacgtggtcaccgtccaaggcccggctgcacctccagggaagatccaacgcatggcggccacaggtcaacaaccctaaggagtggctccaggtggacttccagaaaaccatgaaggtcaccggagtcacaacccagggagtgaagtcgctgctgacttctatgtacgtcaaggagttcctgatctccagcagccaggacgggcaccagtggaccctgttcttccaaaatggaaaggtcaaggtgtttcagggcaatcaggattcattcaccccggtggtgaactcccttgatccacccctcctgacccgctaccttcgcatccacccacagtcctgggtgcaccagatcgcgctgaggatggaggtcctgggatgcgaagcccaggacctgtactga

SEQ ID NO: 13 - DNA, 1734 bp, synthetic construct; identical to SEQ ID NO: 11
  misc_feature 1..1734: note = coFVIII-26-NT58
  source 1..1734: mol_type = other DNA

SEQ ID NO: 14 - DNA, 2583 bp, synthetic construct
  misc_feature 1..2583: note = coFVIII-26-CT
  source 1..2583: mol_type = other DNA
ttcgacgagaacaggagctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 15 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 16 - DNA, 4371 bp, synthetic construct
  misc_feature 1..4371: note = BDD-FVIII (non-optimized; "parental"), Nucleotide Sequence
  source 1..4371: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 17 - protein, 1438 aa, synthetic construct
  REGION 1..1438: note = BDD-FVIII (non-optimized; "parental"), Amino Acid Sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 18 - DNA, 450 bp, synthetic construct
  misc_feature 1..450: note = XTEN nucleotide sequence
  source 1..450: mol_type = other DNA
ggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagc

SEQ ID NO: 19 - DNA, 4824 bp, synthetic construct
  misc_feature 1..4824: note = coFVIII-52-XTEN
  source 1..4824: mol_type = other DNA
atgcaaatcgaactgagcacctgtttcttcctctgcctgctgagattctgtttctccgcgacccgccgatactacctgggagcagtggagctctcctgggattacatgcagagcgaccttggggagctgcccgtggatgccaggttccctccccgggtgccaaagtcgtttccgttcaacacctccgtggtgtacaagaaaactctgttcgtggagttcaccgaccacctgttcaatatcgccaagcccagacctccctggatggggctgttgggacctaccatccaagcggaggtgtacgacactgtggtcatcactctgaagaacatggcctcgcatcccgtgtccctgcacgccgtgggagtgtcttactggaaagcgtccgagggggccgaatacgacgaccagacctcgcagagagaaaaggaagatgacaaggtgttcccaggaggatcgcacacctacgtgtggcaagtgttgaaggagaacggcccaatggcctccgacccgctgtgcctgacctactcgtacctgtcccacgtggacctcgtgaaggacctcaactcgggactgattggagccctgctggtctgcagggaaggctcactggcgaaagaaaagactcagaccttgcacaagttcattctgctgttcgctgtgttcgacgaggggaagtcgtggcacagcgagactaagaactccctgatgcaagatagagatgccgcctccgcccgggcctggcctaagatgcacaccgtgaacggttacgtgaaccgctccctccctggcctgattggatgccaccggaagtccgtgtactggcacgtgatcgggatggggaccacccccgaggtgcacagcatcttcctggaaggtcacacatttctcgtgcgcaaccaccggcaggcctccctggaaatcagccccattaccttcctcactgcccagactctgctgatggacctgggacagttcctgctgttctgccatatctcctcccaccaacatgacggaatggaggcatacgtgaaggtcgattcctgccctgaggaaccccagctccgcatgaagaacaatgaggaagccgaggactacgacgacgacctgacggatagcgagatggatgtggtccggttcgatgacgataacagcccttccttcatccaaattcgctcggtggcaaagaagcaccccaagacctgggtgcattacattgcggcggaagaagaggactgggattatgccccgcttgtcctcgctcctgacgaccggagctacaagagccagtacctgaacaacggtccacagaggatcggtagaaagtacaagaaggtccgcttcatggcctataccgacgaaaccttcaaaactagagaggccatccaacacgaatccggcatcctgggcccgctcttgtacggagaagtcggcgacacccttctcattatcttcaagaaccaggcttcccggccgtacaacatctatccgcatgggatcactgacgtgcgcccactgtactcgcggcgcctgcccaagggtgtcaaacacctgaaggattttccgatccttccgggagaaatcttcaagtacaagtggaccgtgaccgtggaagatggcccaactaagtctgaccctagatgcctcacccgctactactcatccttcgtcaacatggagcgcgacctggccagcggactgatcggcccgctgctgatttgctacaaggaatcagtggaccaacggggaaaccagatcatgtcggataagaggaacgtcatcctcttctccgtgtttgacgaaaaccggtcgtggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 20 - DNA, 4824 bp, synthetic construct
  misc_feature 1..4824: note = coFVIII-1-XTEN
  source 1..4824: mol_type = other DNA
atgcagattgagctgtctacttgctttttcctgtgcctgctgaggttttgcttttccgctacacgaaggtattatctgggggctgtggaactgtcttgggattacatgcagagtgacctgggagagctgccagtggacgcaaggtttccccctagagtccctaagtcattccccttcaacactagcgtggtctacaagaaaacactgttcgtggagtttactgatcacctgttcaacatcgcaaagcctaggccaccctggatgggactgctggggccaacaatccaggccgaggtgtacgacaccgtggtcattacacttaagaacatggcctcacaccccgtgagcctgcatgctgtgggcgtcagctactggaaggcttccgaaggagcagagtatgacgatcagacttcccagagagaaaaagaggacgataaggtgtttcctggcggatctcatacctacgtgtggcaggtcctgaaagagaatggccctatggcctccgaccctctgtgcctgacctactcttatctgagtcacgtggacctggtcaaggatctgaacagcggcctgatcggagccctgctggtgtgcagggaaggaagcctggctaaggagaaaacccagacactgcataagttcattctgctgttcgccgtgtttgacgaagggaaatcatggcacagcgagacaaagaatagtctgatgcaggacagggatgccgcttcagccagagcttggcccaaaatgcacactgtgaacggctacgtcaatcgctcactgcctgggctgatcggctgccaccgaaagagcgtgtattggcatgtcatcgggatgggcaccacacctgaagtgcactccattttcctggagggacatacctttctggtccgcaaccaccgacaggcttccctggagatctctccaattaccttcctgacagcacagactctgctgatggacctggggcagttcctgctgttttgccacatcagctcccaccagcatgatggcatggaggcttacgtgaaagtggactcttgtcccgaggaacctcagctgcggatgaagaacaatgaggaagcagaagactatgacgatgacctgaccgactccgagatggatgtggtccgattcgatgacgataacagcccctcctttatccagattagatctgtggccaagaaacaccctaagacatgggtccattacatcgcagccgaggaagaggactgggattatgcaccactggtgctggcaccagacgatcgctcctacaaatctcagtatctgaacaatgggccacagaggattggcagaaagtacaagaaagtgcggttcatggcatataccgatgagaccttcaagactcgcgaagccatccagcacgagagcggcatcctgggaccactgctgtacggagaagtgggagacaccctgctgatcattttcaagaaccaggccagccggccttacaatatctatccacatgggattacagatgtgcgccctctgtacagcaggagactgccaaagggcgtcaaacacctgaaggacttcccaatcctgcccggagaaatcttcaagtacaagtggactgtcaccgtcgaggatggccccactaagagcgaccctcggtgcctgacccgctactattctagtttcgtgaatatggaaagagatctggcaagcggactgatcggaccactgctgatttgttacaaagagagcgtggatcagagaggcaaccagatcatgtccgacaagcggaatgtgattctgttcagtgtctttgacgaaaacaggtcatggtacctgaccgagaacatccagagattcctgcctaatccagctggggtgcagctggaagatcctgagtttcaggcatctaacatcatgcatagtattaatggctacgtgttcgacagtttgcagctgagcgtgtgcctgcacgaggtcgcttactggtatatcctgagcattggggcacagacagatttcctgagcgtgttcttttccggctacacttttaagcataaaatggtctatgaggacacactgactctgttccccttcagcggcgaaaccgtgtttatgagcatggagaatcccggactgtggattctggggtgccacaacagcgatttcagaaatcgcggaatgactgccctgctgaaagtgtcaagctgtgacaagaacaccggggactactatgaagattcatacgaggacatcagcgcatatctgctgtccaaaaacaatgccattgaaccccggtcttttagtcagaatggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagccctccagtgctgaagcggcaccagcgcgagatcacccgcactaccctgcagagtgatcaggaagagatcgactacgacgatacaatttctgtggaaatgaagaaagaggacttcgatatctatgacgaagatgagaaccagagtcctcgatcattccagaagaaaaccaggcattactttattgccgcagtggagcggctgtgggattatggcatgtcctctagtcctcacgtgctgcgaaatagggcccagtcaggaagcgtcccacagttcaagaaagtggtcttccaggagtttacagacgggtcctttactcagccactgtacaggggcgaactgaacgagcacctgggactgctggggccctatatcagagcagaagtggaggataacattatggtcaccttcagaaatcaggcctctcggccttacagtttttattcaagcctgatctcttacgaagaggaccagcgacagggagctgaaccacgaaaaaacttcgtgaagcctaatgagaccaaaacatacttttggaaggtgcagcaccatatggccccaacaaaagacgagttcgattgcaaggcatgggcctatttttctgacgtggatctggagaaggacgtgcacagtggcctgattggcccactgctggtgtgccatactaacaccctgaatccagcccacggccggcaggtcactgtccaggagttcgctctgttctttaccatctttgatgagacaaagagctggtacttcaccgaaaacatggagcgaaattgcagggctccatgtaacattcagatggaagaccccacattcaaggagaactaccgctttcatgctatcaatggatacatcatggatactctgcccgggctggtcatggcacaggaccagagaatccggtggtatctgctgagcatgggcagcaacgagaatatccactcaattcatttcagcgggcacgtgtttactgtcaggaagaaagaagagtacaagatggccctgtacaacctgtatcccggcgtgttcgaaaccgtcgagatgctgcctagcaaggccggaatctggagagtggaatgcctgattggagagcacctgcatgctgggatgtctaccctgtttctggtgtacagtaataagtgtcagacacccctgggaatggcatccgggcatatcagggatttccagattaccgcatctggacagtacggacagtgggcacctaagctggctagactgcactattccggatctatcaacgcttggtccacaaaagagcctttctcttggattaaggtggacctgctggccccaatgatcattcatggcatcaaaactcagggagctcggcagaagttctcctctctgtacatctcacagtttatcatcatgtacagcctggatgggaagaaatggcagacataccgcggcaatagcacaggaactctgatggtgttctttggcaacgtggacagcagcggaatcaagcacaacattttcaatccccctatcattgctagatacatccggctgcacccaacccattattctattcgaagtacactgaggatggaactgatgggatgcgatctgaacagttgttcaatgcccctggggatggagtccaaggcaatctctgacgcccagattaccgccagctcctacttcactaatatgtttgctacctggagcccttccaaagcaagactgcacctgcaaggccgcagcaacgcatggcgaccacaggtgaacaatcccaaggagtggttgcaggtcgattttcagaaaactatgaaggtgaccggggtcacaactcagggcgtgaaaagtctgctgacctcaatgtacgtcaaggagttcctgatctctagttcacaggacggacatcagtggacactgttctttcagaacgggaaggtgaaagtcttccagggcaatcaggattcctttacacctgtggtcaacagtctagaccctccactgctgaccagatacctgagaatccaccctcagtcctgggtgcaccagattgccctgagaatggaagtgctgggatgcgaggcccaggatctgtactga

SEQ ID NO: 21-31 - skipped (11 entries)

SEQ ID NO: 32 - DNA, 13 bp, synthetic construct
  misc_feature 1..13: note = kozak consensus sequence
  source 1..13: mol_type = other DNA
gccgccaccatgc

SEQ ID NO: 33 - protein, 32 aa, synthetic construct
  REGION 1..32: note = CTP peptide
DPRFQDSSSSKAPPPSLPSPSRLPGPSDTPIL

SEQ ID NO: 34 - protein, 28 aa, synthetic construct
  REGION 1..28: note = CTP peptide
SSSSKAPPPSLPSPSRLPGPSDTPILPQ

SEQ ID NO: 35 - protein, 11 aa, synthetic construct
  REGION 1..11: note = albumin-binding peptide core sequence
DICLPRWGCLW

SEQ ID NO: 36 - protein, 20 aa, synthetic construct
  REGION 1..20: note = PAS sequence
ASPAAPAPASPAAPAPSAPA

SEQ ID NO: 37 - protein, 20 aa, synthetic construct
  REGION 1..20: note = PAS sequence
AAPASPAPAAPSAPAPAAPS

SEQ ID NO: 38 - protein, 20 aa, synthetic construct
  REGION 1..20: note = PAS sequence
APSSPSPSAPSSPSPASPSS

SEQ ID NO: 39 - protein, 19 aa, synthetic construct
  REGION 1..19: note = PAS sequence
APSSPSPSAPSSPSPASPS

SEQ ID NO: 40 - protein, 20 aa, synthetic construct
  REGION 1..20: note = PAS sequence
SSPSAPSPSSPASPSPSSPA

SEQ ID NO: 41 - protein, 24 aa, synthetic construct
  REGION 1..24: note = PAS sequence
AASPAAPSAPPAAASPAAPSAPPA

SEQ ID NO: 42 - protein, 20 aa, synthetic construct
  REGION 1..20: note = PAS sequence
ASAAAPAAASAAASAPSAAA

SEQ ID NO: 43 - DNA, 23 bp, synthetic construct
  misc_feature 1..23: note = miR142 target
  source 1..23: mol_type = other DNA
tccataaagtaggaaacactaca

SEQ ID NO: 44 - protein, 2332 aa, Homo sapiens; identical to SEQ ID NO: 15

SEQ ID NO: 45 - protein, 4 aa, synthetic construct
  REGION 1..4: note = amino acids 233-236 of human IgG1
ELLG

SEQ ID NO: 46 - protein, 42 aa, synthetic construct
  REGION 1..42: note = XTEN AE42-4, protein sequence
GAPGSPAGSPTSTEEGTSESATPESGPGSEPATSGSETPASS

SEQ ID NO: 47 - DNA, 126 bp, synthetic construct
  misc_feature 1..126: note = XTEN AE42-4, DNA sequence
  source 1..126: mol_type = other DNA
ggcgcgccaggttctcctgctggctcccccacctcaacagaagaggggacaagcgaaagcgctacgcctgagagtggccctggctctgagccagccacctccggctctgaaacccctgcctcgagc

SEQ ID NO: 48 - protein, 144 aa, synthetic construct
  REGION 1..144: note = XTEN AE144-2A, protein sequence
TSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPG

SEQ ID NO: 49 - DNA, 450 bp, synthetic construct
  misc_feature 1..450: note = XTEN AE144-2A, DNA sequence
  source 1..450: mol_type = other DNA
ggcgcgccaaccagtacggagccgtccgaggggagcgcaccaggaagcccggctgggagcccgacttctaccgaagagggtacatctaccgaaccaagtgaaggttcagcaccaggcacctcaacagaaccctctgagggctcggcgcctggtacaagtgagtccgccaccccagaatccgggcctgggacaagcacagaaccttcggaagggagtgcccctggaacatccgaatcggcaaccccagaatcagggccaggatctgagcccgcgacttcgggctccgagacgcctgggacatccaccgagccctccgaaggatcagccccaggcaccagcacggagccctctgagggaagcgcacctggtaccagcgaaagcgcaactcccgaatcaggtcccggtacgagcgagtcggcgaccccggagagcgggccaggtgcctcgagc

SEQ ID NO: 50 - protein, 144 aa, synthetic construct
  REGION 1..144: note = XTEN AE144-3B, protein sequence
SPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPG

SEQ ID NO: 51 - DNA, 450 bp, synthetic construct
  misc_feature 1..450: note = XTEN AE144-3B, DNA sequence
  source 1..450: mol_type = other DNA
ggcgcgccaagtcccgctggaagcccaactagcaccgaagaggggacctcagagtccgccacccccgagtccggccctggctctgagcctgccactagcggctccgagactcctggcacatccgaaagcgctacacccgagagtggacccggcacctctaccgagcccagtgagggctccgcccctggaacaagcaccgagcccagcgaaggcagcgccccagggacctccacagagcccagtgaaggcagtgctcctggcaccagcaccgaaccaagcgagggctctgcacccgggacctccaccgagccaagcgaaggctctgcccctggcacttccaccgagcccagcgaaggcagcgcccctgggagccccgctggctctcccaccagcactgaggagggcacatctaccgaaccaagtgaaggctctgcaccaggtgcctcgagc

SEQ ID NO: 52 - protein, 144 aa, synthetic construct
  REGION 1..144: note = XTEN AE144-4A, protein sequence
TSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPG

SEQ ID NO: 53 - DNA, 450 bp, synthetic construct
  misc_feature 1..450: note = XTEN AE144-4A, DNA sequence
  source 1..450: mol_type = other DNA
ggcgcgccaacgtccgaaagtgctacccctgagtcaggccctggtagtgagcctgccacaagcggaagcgaaactccggggacctcagagtctgccactcccgaatcggggccaggctctgaaccggccacttcagggagcgaaacaccaggaacatcggagagcgctaccccggagagcgggccaggaactagtactgagcctagcgagggaagtgcacctggtacaagcgagtccgccacacccgagtctggccctggctctccagcgggctcacccacgagcactgaagagggctctcccgctggcagcccaacgtcgacagaagaaggatcaccagcaggctcccccacatcaacagaggagggtacatcagaatctgctactcccgagagtggacccggtacctccactgagcccagcgaggggagtgcaccaggtgcctcgagc

SEQ ID NO: 54 - protein, 144 aa, synthetic construct
  REGION 1..144: note = XTEN AE144-5A, protein sequence
TSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEG

SEQ ID NO: 55 - DNA, 450 bp, synthetic construct; identical to SEQ ID NO: 18
  misc_feature 1..450: note = XTEN AE144-5A, DNA sequence
  source 1..450: mol_type = other DNA

SEQ ID NO: 56 - protein, 144 aa, synthetic construct
  REGION 1..144: note = XTEN AE144-6B, protein sequence
TSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 57 - DNA, 450 bp, synthetic construct
  misc_feature 1..450: note = XTEN AE144-6B, DNA sequence
  source 1..450: mol_type = other DNA
ggcgcgccaacatctaccgagccttccgaaggctctgcccctgggacctcagaatctgcaacccctgaaagcggccctggaacctccgaaagtgccactcccgagagcggcccagggacaagcgagtcagcaacccctgagtctggacccggcagcgagcctgcaacctctggctcagagactcccggctcagaacccgctacctcaggctccgagacacccggctctcctgctgggagtcccacttccaccgaggaaggaacatccactgagcctagtgagggctctgcccctggaaccagcacagagccaagtgagggcagtgcaccaggatccgagccagcaaccagcgggtccgagactcccgggacctctgagtctgccaccccagagagcggacccggcacttcaaccgagccctccgaaggatcagcaccaggtgcctcgagc

SEQ ID NO: 58 - protein, 144 aa, synthetic construct
  REGION 1..144: note = XTEN AG144-1, protein sequence
PGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSS

SEQ ID NO: 59 - DNA, 450 bp, synthetic construct
  misc_feature 1..450: note = XTEN AG144-1, DNA sequence
  source 1..450: mol_type = other DNA
ggcgcgccacccgggtcgtccccgtcggcgtccaccggaacagggccagggtcatccccgtcagcgtcgactgggacgggacccgggacacccggttcggggactgcatcctcctcgcctggttcgtccaccccgtcaggagccacgggttcgccgggaagcagcccaagcgcatccactggtacagggcctggggcttcaccgggtacttcatccacggggtcaccgggaacgcccggatcggggacggcttcctcatcaccaggatcgtcaacaccctcgggcgcaacgggcagccccggaacccctggttcgggtacggcgtcgtcgagccccggtgcgagcccgggaacaagctcgacaggatcgcctggggcgtcacccggcacgtcgagcacaggcagccccggaacccctggatcgggaaccgcgtcgtcaagcgcctcgagc

SEQ ID NO: 60 - protein, 144 aa, synthetic construct
  REGION 1..144: note = XTEN AG144-A, protein sequence
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSP

SEQ ID NO: 61 - DNA, 450 bp, synthetic construct
  misc_feature 1..450: note = XTEN AG144-A, DNA sequence
  source 1..450: mol_type = other DNA
ggcgcgccaggtgcctcgccgggaacatcatcaactggttcacccgggtcatccccctcggcctcaaccgggacgggtcccggctcatcccccagcgccagcactggaacaggtcctggcactcctggttccggtacggcatcgtcatccccgggaagctcaacaccgtccggagcgacaggatcacctggctcgtcaccttcggcgtcaactggaacggggccaggggcctcacccggaacgtcctcgactgggtcgcctggtacgccgggatcaggaacggcctcatcctcgcctgggtcctcaacgccctcgggtgcgactggttcgccgggaactcctggctcggggacggcctcgtcgtcgcctggggcatcaccggggacgagctccacggggtcccctggagcgtcaccggggacctcctcgacaggtagcccggcctcgagc

SEQ ID NO: 62 - protein, 144 aa, synthetic construct
  REGION 1..144: note = XTEN AG144-B, protein sequence
GTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSP

SEQ ID NO: 63 - DNA, 450 bp, synthetic construct
  misc_feature 1..450: note = XTEN AG144-B, DNA sequence
  source 1..450: mol_type = other DNA
ggcgcgccaggtacaccgggcagcggcacggcttcgtcgtcacccggctcgtccacaccgtcgggagctacgggaagcccaggagcgtcaccgggaacgtcgtcaacggggtcaccgggtacgccaggtagcggcacggccagcagctcgccaggttcatcgaccccgtcgggagcgactgggtcgcccggatcaagcccgtcagcttccactggaacaggacccgggtcgtcgccgtcagcctcaacggggacaggacctggttcatcgacgccgtcaggggcgacaggctcgcccggatcgtcaacaccctcgggggcaacggggagccctggtgcgtcgcctggaacctcatccaccggaagcccgggggcctcgccgggtacgagctccacgggatcgcccggagcgtcccccggaacttcaagcacagggagccctgcctcgagc

SEQ ID NO: 64 - protein, 144 aa, synthetic construct
  REGION 1..144: note = XTEN AG144-C, protein sequence
GTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSP

SEQ ID NO: 65 - DNA, 450 bp, synthetic construct
  misc_feature 1..450: note = XTEN AG144-C, DNA sequence
  source 1..450: mol_type = other DNA
ggcgcgccaggtacacccggatcgggtacagcgtcatcgagccccggtgcgtcacctggtacgtcgagcacggggtcgccaggggcgtcccctgggacgtcctcaacaggctcgcccggtgcgtcacccggcacgtcgtccacgggttcacctggtagctccccttccgcgtccactggcaccgggcctggaactccggggagcggcacagcgagctcgtcgccgggagcatcgcctgggacatcgagcaccgggtcgccaggagcatcgcccggaacatccagcacaggaagccccggcgcgtcgcccgggacatcaagcacaggttccccgggatcgagcacgccgtccggagccactggatcaccagggagctcgacaccttccggcgcaacgggatcgcccggagccagcccgggtacgtcaagcactggctcccctgcctcgagc

SEQ ID NO: 66 - protein, 144 aa, synthetic construct
  REGION 1..144: note = XTEN AG144-F, protein sequence
GSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 67 - DNA, 450 bp, synthetic construct
  misc_feature 1..450: note = XTEN AG144-F, DNA sequence
  source 1..450: mol_type = other DNA
ggcgcgccaggctccagcccctccgcgagcacgggaaccggaccaggttcgtcaccctcagcatcaacggggacgggaccgggggcgtcaccaggaacgtcctccaccggctcgccgggtgcatcacccggaacgtcatcgaccggatcgccagggagctcgacgccatcaggcgcaacaggatcacctggctcaagccctagcgcgtcaaccggcacgggtccgggtgcctcccctggcacgtccagcaccggatcacccggatcgagcccatccgcctcaaccggaaccggacccggtacaccagggtcgggaacagcctcctcgtcaccaggctcctcaaccccctcgggagccacgggttcgcccggttcgtcaacgccttccggagcaactggtagccccggagcatcgccaggaacttcgagcacggggtcgcccgcctcgagc

SEQ ID NO: 68 - DNA, 4374 bp, synthetic construct
  misc_feature 1..4374: note = coFVIII-1 DNA Sequence
  source 1..4374: mol_type = other DNA
atgcagattgagctgtctacttgctttttcctgtgcctgctgaggttttgcttttccgctacacgaaggtattatctgggggctgtggaactgtcttgggattacatgcagagtgacctgggagagctgccagtggacgcaaggtttccccctagagtccctaagtcattccccttcaacactagcgtggtctacaagaaaacactgttcgtggagtttactgatcacctgttcaacatcgcaaagcctaggccaccctggatgggactgctggggccaacaatccaggccgaggtgtacgacaccgtggtcattacacttaagaacatggcctcacaccccgtgagcctgcatgctgtgggcgtcagctactggaaggcttccgaaggagcagagtatgacgatcagacttcccagagagaaaaagaggacgataaggtgtttcctggcggatctcatacctacgtgtggcaggtcctgaaagagaatggccctatggcctccgaccctctgtgcctgacctactcttatctgagtcacgtggacctggtcaaggatctgaacagcggcctgatcggagccctgctggtgtgcagggaaggaagcctggctaaggagaaaacccagacactgcataagttcattctgctgttcgccgtgtttgacgaagggaaatcatggcacagcgagacaaagaatagtctgatgcaggacagggatgccgcttcagccagagcttggcccaaaatgcacactgtgaacggctacgtcaatcgctcactgcctgggctgatcggctgccaccgaaagagcgtgtattggcatgtcatcgggatgggcaccacacctgaagtgcactccattttcctggagggacatacctttctggtccgcaaccaccgacaggcttccctggagatctctccaattaccttcctgacagcacagactctgctgatggacctggggcagttcctgctgttttgccacatcagctcccaccagcatgatggcatggaggcttacgtgaaagtggactcttgtcccgaggaacctcagctgcggatgaagaacaatgaggaagcagaagactatgacgatgacctgaccgactccgagatggatgtggtccgattcgatgacgataacagcccctcctttatccagattagatctgtggccaagaaacaccctaagacatgggtccattacatcgcagccgaggaagaggactgggattatgcaccactggtgctggcaccagacgatcgctcctacaaatctcagtatctgaacaatgggccacagaggattggcagaaagtacaagaaagtgcggttcatggcatataccgatgagaccttcaagactcgcgaagccatccagcacgagagcggcatcctgggaccactgctgtacggagaagtgggagacaccctgctgatcattttcaagaaccaggccagccggccttacaatatctatccacatgggattacagatgtgcgccctctgtacagcaggagactgccaaagggcgtcaaacacctgaaggacttcccaatcctgcccggagaaatcttcaagtacaagtggactgtcaccgtcgaggatggccccactaagagcgaccctcggtgcctgacccgctactattctagtttcgtgaatatggaaagagatctggcaagcggactgatcggaccactgctgatttgttacaaagagagcgtggatcagagaggcaaccagatcatgtccgacaagcggaatgtgattctgttcagtgtctttgacgaaaacaggtcatggtacctgaccgagaacatccagagattcctgcctaatccagctggggtgcagctggaagatcctgagtttcaggcatctaacatcatgcatagtattaatggctacgtgttcgacagtttgcagctgagcgtgtgcctgcacgaggtcgcttactggtatatcctgagcattggggcacagacagatttcctgagcgtgttcttttccggctacacttttaagcataaaatggtctatgaggacacactgactctgttccccttcagcggcgaaaccgtgtttatgagcatggagaatcccggactgtggattctggggtgccacaacagcgatttcagaaatcgcggaatgactgccctgctgaaagtgtcaagctgtgacaagaacaccggggactactatgaagattcatacgaggacatcagcgcatatctgctgtccaaaaacaatgccattgaaccccggtcttttagtcagaatcctccagtgctgaagaggcaccagagggagatcacccgcactaccctgcagagtgatcaggaagagatcgactacgacgatacaatttctgtggaaatgaagaaagaggacttcgatatctatgacgaagatgagaaccagagtcctcgatcattccagaagaaaaccaggcattactttattgccgcagtggagcggctgtgggattatggcatgtcctctagtcctcacgtgctgcgaaatagggcccagtcaggaagcgtcccacagttcaagaaagtggtcttccaggagtttacagacgggtcctttactcagccactgtacaggggcgaactgaacgagcacctgggactgctggggccctatatcagagcagaagtggaggataacattatggtcaccttcagaaatcaggcctctcggccttacagtttttattcaagcctgatctcttacgaagaggaccagcgacagggagctgaaccacgaaaaaacttcgtgaagcctaatgagaccaaaacatacttttggaaggtgcagcaccatatggccccaacaaaagacgagttcgattgcaaggcatgggcctatttttctgacgtggatctggagaaggacgtgcacagtggcctgattggcccactgctggtgtgccatactaacaccctgaatccagcccacggccggcaggtcactgtccaggagttcgctctgttctttaccatctttgatgagacaaagagctggtacttcaccgaaaacatggagcgaaattgcagggctccatgtaacattcagatggaagaccccacattcaaggagaactaccgctttcatgctatcaatggatacatcatggatactctgcccgggctggtcatggcacaggaccagagaatccggtggtatctgctgagcatgggcagcaacgagaatatccactcaattcatttcagcgggcacgtgtttactgtcaggaagaaagaagagtacaagatggccctgtacaacctgtatcccggcgtgttcgaaaccgtcgagatgctgcctagcaaggccggaatctggagagtggaatgcctgattggagagcacctgcatgctgggatgtctaccctgtttctggtgtacagtaataagtgtcagacacccctgggaatggcatccgggcatatcagggatttccagattaccgcatctggacagtacggacagtgggcacctaagctggctagactgcactattccggatctatcaacgcttggtccacaaaagagcctttctcttggattaaggtggacctgctggccccaatgatcattcatggcatcaaaactcagggagctcggcagaagttctcctctctgtacatctcacagtttatcatcatgtacagcctggatgggaagaaatggcagacataccgcggcaatagcacaggaactctgatggtgttctttggcaacgtggacagcagcggaatcaagcacaacattttcaatccccctatcattgctagatacatccggctgcacccaacccattattctattcgaagtacactgaggatggaactgatgggatgcgatctgaacagttgttcaatgcccctggggatggagtccaaggcaatctctgacgcccagattaccgccagctcctacttcactaatatgtttgctacctggagcccttccaaagcaagactgcacctgcaaggccgcagcaacgcatggcgaccacaggtgaacaatcccaaggagtggttgcaggtcgattttcagaaaactatgaaggtgaccggggtcacaactcagggcgtgaaaagtctgctgacctcaatgtacgtcaaggagttcctgatctctagttcacaggacggacatcagtggacactgttctttcagaacgggaaggtgaaagtcttccagggcaatcaggattcctttacacctgtggtcaacagtctagaccctccactgctgaccagatacctgagaatccaccctcagtcctgggtgcaccagattgccctgagaatggaagtgctgggatgcgaggcccaggatctgtactga

SEQ ID NO: 69 - DNA, 577 bp, synthetic construct
  misc_feature 1..577: note = ET Promoter, DNA sequence
  source 1..577: mol_type = other DNA
ctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgccaccatgg

SEQ ID NO: 70 - DNA, 4374 bp, synthetic construct; identical to SEQ ID NO: 1
  misc_feature 1..4374: note = coFVIII-5
  source 1..4374: mol_type = other DNA

SEQ ID NO: 71 - DNA, 4374 bp, synthetic construct
  misc_feature 1..4374: note = coFVIII-6
  source 1..4374: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 72 - DNA, 4824 bp, synthetic construct
  misc_feature 1..4824: note = coFVIII-6-XTEN
  source 1..4824: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 73 - protein, 12 aa, synthetic construct
  REGION 1..12: note = AD motif
GESPGGSSGSES

SEQ ID NO: 74 - protein, 12 aa, synthetic construct
  REGION 1..12: note = AD motif
GSEGSSGPGESS

SEQ ID NO: 75 - protein, 12 aa, synthetic construct
  REGION 1..12: note = AD motif
GSSESGSSEGGP

SEQ ID NO: 76 - protein, 12 aa, synthetic construct
  REGION 1..12: note = AD motif
GSGGEPSESGSS

SEQ ID NO: 77 - protein, 12 aa, synthetic construct
  REGION 1..12: note = AE, AM motif
GSPAGSPTSTEE

SEQ ID NO: 78 - protein, 12 aa, synthetic construct
  REGION 1..12: note = AE, AM, AQ motif
GSEPATSGSETP

SEQ ID NO: 79 - protein, 12 aa, synthetic construct
  REGION 1..12: note = AE, AM, AQ motif
GTSESATPESGP

SEQ ID NO: 80 - protein, 12 aa, synthetic construct
  REGION 1..12: note = AE, AM, AQ motif
GTSTEPSEGSAP

SEQ ID NO: 81 - protein, 12 aa, synthetic construct
  REGION 1..12: note = AF, AM motif
GSTSESPSGTAP

SEQ ID NO: 82 - protein, 12 aa, synthetic construct
  REGION 1..12: note = AF, AM motif
GTSTPESGSASP

SEQ ID NO: 83 - protein, 12 aa, synthetic construct
  REGION 1..12: note = AF, AM motif
GTSPSGESSTAP

SEQ ID NO: 84 - protein, 12 aa, synthetic construct
  REGION 1..12: note = AF, AM motif
GSTSSTAESPGP

SEQ ID NO: 85 - protein, 12 aa, synthetic construct
  REGION 1..12: note = AG, AM motif
GTPGSGTASSSP

SEQ ID NO: 86 - protein, 12 aa, synthetic construct
  REGION 1..12: note = AG, AM motif
GSSTPSGATGSP

SEQ ID NO: 87 - protein, 12 aa, synthetic construct
  REGION 1..12: note = AG, AM motif
GSSPSASTGTGP

SEQ ID NO: 88 - protein, 12 aa, synthetic construct
  REGION 1..12: note = AG, AM motif
GASPGTSSTGSP

SEQ ID NO: 89 - protein, 12 aa, synthetic construct
  REGION 1..12: note = AQ motif
GEPAGSPTSTSE

SEQ ID NO: 90 - protein, 12 aa, synthetic construct
  REGION 1..12: note = AQ motif
GTGEPSSTPASE

SEQ ID NO: 91 - protein, 12 aa, synthetic construct
  REGION 1..12: note = AQ motif
GSGPSTESAPTE

SEQ ID NO: 92 - protein, 12 aa, synthetic construct
  REGION 1..12: note = AQ motif
GSETPSGPSETA

SEQ ID NO: 93 - protein, 12 aa, synthetic construct
  REGION 1..12: note = AQ motif
GPSETSTSEPGA

SEQ ID NO: 94 - protein, 12 aa, synthetic construct
  REGION 1..12: note = AQ motif
GSPSEPTEGTSA

SEQ ID NO: 95 - protein, 12 aa, synthetic construct
  REGION 1..12: note = BC motif
GSGASEPTSTEP

SEQ ID NO: 96 - protein, 12 aa, synthetic construct
  REGION 1..12: note = BC motif
GSEPATSGTEPS

SEQ ID NO: 97 - protein, 12 aa, synthetic construct
  REGION 1..12: note = BC motif
GTSEPSTSEPGA

SEQ ID NO: 98 - protein, 12 aa, synthetic construct
  REGION 1..12: note = BC motif
GTSTEPSEPGSA

SEQ ID NO: 99 - protein, 12 aa, synthetic construct
  REGION 1..12: note = BD motif
GSTAGSETSTEA

SEQ ID NO: 100 - protein, 12 aa, synthetic construct
  REGION 1..12: note = BD motif
GSETATSGSETA

SEQ ID NO: 101 - protein, 12 aa, synthetic construct
  REGION 1..12: note = BD motif
GTSESATSESGA

SEQ ID NO: 102 - protein, 12 aa, synthetic construct
  REGION 1..12: note = BD motif
GTSTEASEGSAS

SEQ ID NO: 103 - protein, 1607 aa, synthetic construct
  REGION 1..1607: note = coFVIII-6-XTEN Protein Sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGAPTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY